(19) United States
(12) Patent Application Publication
(10) Pub. No.: US 2021/0024593 A1
(43) Pub. Date: Jan. 28, 2021

(54) MODIFIED HEAT SHOCK PROTEINS

(71) Applicant: Rubicon Biotechnology, LLC, Lake Forest, CA (US)

(72) Inventor: Missag H. PARSEGHIAN, Lake Forest, CA (US)

(21) Appl. No.: 16/641,013

(22) PCT Filed: Aug. 24, 2018
(86) PCT No.: PCT/US2018/047976

§ 371 Date: Feb. 21, 2020

Related U.S. Application Data
(60) Provisional application No. 62/549,860, filed on Aug. 24, 2017.
(60) Provisional application No. 62/615,915, filed on Jan. 10, 2018.

(51) Int. Cl. C07K 14/47; C07K 1/107; A61K 9/127; A61K 47/68; A61K 47/64

(52) U.S. Cl. CPC C07K 14/47; C07K 1/1077; A61K 38/00; A61K 47/6835; A61K 47/645; A61K 9/127

(57) ABSTRACT

The present disclosure generally relates to modified heat shock protein compositions that improve intracellular performance when delivered across the plasma and/or nuclear membranes. Also provided are methods for treating ocular, neurological, muscular, hepatic, renal, integumentary, cardiovascular and pulmonary conditions with the modified heat shock proteins are disclosed herein.

### CROSS-REFERENCE TO RELATED APPLICATIONS

The present application claims priority to U.S. Provisional Patent Application Ser. No. 62/549,860, filed on Aug. 24, 2017; and U.S. Provisional Patent Application Ser. No. 62/615,915, filed on Jan. 10, 2018. The contents of the above-referenced applications are hereby expressly incorporated by reference in their entireties.

### STATEMENT REGARDING FEDERALLY SPONSORED R&D

This invention was made with government support under grant no. R21ES024028 awarded by the National Institutes of Health. The government has certain rights in the invention.

### FIELD

The present disclosure relates generally to the field of molecular biology and medicine. More particularly, provided herein are novel modified heat shock proteins, pharmaceutical compositions containing the same, methods useful for producing such modified heat shock proteins, as well as methods for treating a disease or health condition in a subject.

### BACKGROUND

The body's natural response to stress or trauma includes heat shock protein (HSP) induction to combat the acute effects of protein denaturation and aggregation and the chronic effects of apoptosis and secondary necrosis. Heat shock proteins (HSPs), such as HSP72, help prevent protein misfolding within the cellular milieu by binding and refolding those proteins which are improperly assembled or in the process of denaturing due to cell stress. HSP72 also inhibits apoptosis that occurs through at least three different pathways (the ATP-dependent apoptosome, ATP-independent AIF and the NF-κB pathways). Furthermore, HSP72 and a related protein, BiP, are integral components of three stress sensing pathways in the endoplasmic reticulum (ER). These “ER stress” sensors include: inositol-requiring kinase-1α (IRE1α), activating transcription factor-6 (ATF6), and protein kinase RNA (PKR)-like ER kinase (PERK). It is generally believed that if continuous ER stress results in an unfolded protein response (UPR), HSP72 directly binds the cytosolic domain of the IRE1α ER membrane protein, enhancing the pro-survival and stress adaptation effects of the IRE1α pathway. It is generally believed that prolonged activation of another UPR pathway, PERK, left unchecked by HSP72 and the IRE1α pathway leads to cell death. Natural HSP induction can take several hours while a patient is in jeopardy of permanent tissue damage; hence, exogenous HSPs delivered to the target tissue has been the subject of investigation and shown promise in pre-clinical studies. Rapid delivery of exogenous HSPs has demonstrated greater cell viability and reduced physiological trauma in tissues damaged by oxidative stress, hypoxia, cardiovascular or neurovascular occlusion, toxic inhalation, and other mechanical, acoustic, radiological, electromagnetic and chemical traumas.

In order for HSPs to be used as cytoprotectants when cells are subjected to stress in a wide range of traumas, large scale commercial production is desired. In addition, HSPs may need commercial production in suitable eukaryotic cells to maintain proper structural folding and achieve quantities that are economically feasible for development as a therapeutic. Production and secretion from eukaryotic cells (e.g. mammalian, insect or fungal) results in N-linked glycosylation of key sites that affect protein efficacy. Furthermore, modifications to the carbohydrate structures when shifting production of the same HSP from one system (e.g. fungal) to another (e.g. mammalian) can significantly and often negatively affect efficacy. Therefore, there is a need of solution that can overcome the foregoing problems and provides functional HSPs.

### SUMMARY

In one aspect, the disclosure disclosed herein relates to a modified heat shock protein or fragment thereof in which one or more of N-glycosylation sites is modified such that N-glycosylation at the modified site is substantially reduced as compared to N-glycosylation at the corresponding unmodified site.

Implementations of embodiments of the modified heat shock protein or fragment thereof according to the present disclosure can include one or more of the following features. In some embodiments, the one or more of N-glycosylation sites comprises an amino acid sequence of N—X—S/T/C, wherein X is any amino acid except proline. In some embodiments, one or more of acid-labile cleavage sites and/or one or more of alkaline-labile cleavage sites is modified such that cleavage at the modified cleavage site at or under pH 4, 5, 6, or 7; or at or above pH 7, 8, 9, 10, 11 or 12 is substantially reduced as compared to cleavage at the corresponding unmodified cleavage site. In some embodiments, the one or more of acid-labile cleavage site comprises an amino acid sequence of D-P. In some embodiments, the one or more of alkaline-labile cleavage site comprises an amino acid sequence of N-P, N-L, N-S or N-T.

In some embodiments, the modified heat shock protein is selected from the group consisting of modified chaperonins, modified HSP27, modified HSP40, modified HSP60, modified HSP70, modified HSP90, modified HSP105/110 and modified small heat shock proteins.

In some embodiments, all N-glycosylation sites are modified such that there is no N-glycosylation in the modified heat shock protein or fragment thereof In some embodiments, N-glycosylation in the modified heat shock protein or fragment thereof is reduced by about 10%, about 20%, about 30%, about 40%, about 50%, about 60%, about 70%, about 80%, about 90%, about 95%, about 99%. or about 100% as compared to N-glycosylation in the corresponding unmodified heat shock protein or fragment thereof. In some embodiments, about 10%, about 20%, about 30%, about 40%, about 50%, about 60%, about 70%, about 80%, about 90%, about 95%, about 99%, or about 100% of the total number of the N-glycosylation sites is modified in the heat shock protein or fragment thereof.

In some embodiments, all acid-labile cleavage sites are modified such that there is no cleavage of the modified heat shock protein or fragment thereof at or under pH 4, 5, 6, or 7. In some embodiments, all alkaline-labile cleavage sites are modified such that there is no cleavage of the modified heat shock protein or fragment thereof at or above pH 7, 8, 9, 10, 11 or 12. In some embodiments, all acid-labile and alkaline-labile cleavage sites are modified such that there is no cleavage of the modified heat shock protein or fragment thereof at or under pH 4, 5, 6, or 7 or at or above pH 7, 8, 9, 10, 11 or 12. In some embodiments, cleavage at or under pH 4, 5, 6, or 7 or at or above pH 7, 8, 9, 10, 11 or 12 in the modified heat shock protein or fragment thereof is reduced by about 10%, about 20%, about 30%, about 40%, about 50%, about 60%, about 70%, about 80%, about 90%, about 95%, about 99%, or about 100% as compared to cleavage in the corresponding unmodified heat shock protein or fragment thereof.

In some embodiments, about 10%, about 20%, about 30%, about 40%, about 50%, about 60%, about 70%, about 80%, about 90%, about 95%, about 99%, or about 100% of the total number of the acid-labile and/or alkaline-labile sites is modified in the heat shock protein or fragment thereof. In some embodiments, about 10%, about 20%, about 30%, about 40%, about 50%, about 60%, about 70%, about 80%, about 90%, about 95%, about 99%, or about 100% of the total number of the N-glycosylation sites is modified; and about 10%, about 20%, about 30%, about 40%, about 50%, about 60%, about 70%, about 80%, about 90%, about 95%, about 99%, or about 100% of the total number of the acid-labile and/or alkaline-labile sites is modified in the heat shock protein or fragment thereof

In some embodiments, all N-glycosylation sites are modified but not all acid-labile cleavage sites and not all alkaline-labile cleavage sites are modified in the heat shock protein or fragment thereof In some embodiments, all N-glycosylation sites and all acid-labile cleavage sites are modified but not all alkaline-labile cleavage sites are modified in the heat shock protein or fragment thereof. In some embodiments, all N-glycosylation sites and all alkaline-labile cleavage sites are modified but not all acid-labile cleavage sites are modified in the heat shock protein or fragment thereof. In some embodiments, all N-glycosylation sites and all acid-labile cleavage sites and all alkaline-labile cleavage sites are modified in the heat shock protein or fragment thereof.

In one aspect, some embodiments of the disclosure relate to a pharmaceutical composition containing a modified heat shock protein or fragment thereof disclosed herein and a pharmaceutically acceptable excipient. In some embodiments, the pharmaceutical composition is formulated into a liposome.

In one aspect, some embodiments of the disclosure relate a conjugate compound having any of the modified heat shock protein or fragment thereof disclosed herein and an additional compound. In some embodiments, the additional compound is a peptide that is capable of transporting the modified heat shock protein or fragment thereof across a lipid bilayer. In some embodiments, the additional compound is an antibody or fragment thereof In some embodiments, the antibody or fragment thereof is capable of binding to a molecule present inside or outside a cell. In some embodiments, the molecule present inside a cell is DNA, RNA or protein present inside a cell. In some embodiments, the molecule is located outside a cell. In some embodiments, the antibody is 3E10. In some embodiments, the 3E10 antibody has an amino acid sequence selected from the group consisting of a murine sequence, a humanized sequence, a fully human analog and any modified form thereof. In some embodiments, the additional compound is selected from the group consisting of histones and fragments thereof, high mobility group proteins (HMGs) and fragments thereof, transcription factors and fragments thereof and poly-cation sequences having a plurality of lysine and/or arginine and fragments thereof. In some embodiments, the additional compound is selected from the group consisting of phospholipid binding proteins and fragments thereof. In some embodiments, the phospholipid binding proteins and fragments thereof are selected from the group consisting of annexins, lactadherin, sphingomyelin, apolipoprotein-H (β-2-glycoprotein-1), T-cell immunoglobulin mucin domain (TIM) receptors, galectins, and fragments of any thereof. In some embodiments, the additional compound is selected from the group consisting of cell-penetrating peptides and fragments thereof In some embodiments, the cell-penetrating peptides and fragments thereof are selected from the group consisting of TAT proteins of HIV and Antennapedia proteins from insects and fragments thereof. In some embodiments, the additional compound is selected from the group consisting of peptides and fragments thereof that bind to ions transported across the membrane.

In one aspect, some embodiments of the disclosure relate to a pharmaceutical composition comprising a conjugate disclosed herein and a pharmaceutically acceptable excipient. In some embodiments, the pharmaceutical composition formulated into a liposome.

In one aspect, some embodiments of the disclosure relate to a method of making a modified heat shock protein or fragment thereof as described herein. The method includes modifying at least one or more of N-glycosylation sites such that N-glycosylation at the modified site is substantially reduced as compared to N-glycosylation at the corresponding unmodified site. In some embodiments, the method includes modifying at least one or more of acid-labile cleavage sites such that cleavage at the modified cleavage site at or under pH 4, 5, 6, or 7 is substantially reduced as compared to cleavage at the corresponding unmodified cleavage site. In some embodiments, the method includes modifying at least one or more of alkaline-labile cleavage sites such that cleavage at the modified cleavage site at or above pH 7, 8, 9, 10, 11 or 12 is substantially reduced as compared to cleavage at the corresponding unmodified cleavage site. In some embodiments, the method disclosed herein includes modifying at least one or more of acid-labile cleavage sites and at least one or more of alkaline-labile cleavage sites such that cleavage at the modified cleavage site at or under pH 4, 5, 6, or 7 or at or above pH 7, 8, 9, 10, 11 or 12 is substantially reduced as compared to cleavage at the corresponding unmodified cleavage site.

In one aspect, some embodiments of the disclosure relate to a method of treating a disease or condition in a subject. The method includes administering a pharmaceutically effective amount of any of the modified heat shock protein or fragment thereof disclosed herein to the subject in need of the treatment. In some embodiments, the disease or condition is selected from the group consisting of ocular, neurological, muscular, hepatic, renal, integumentary, cardiovascular and pulmonary conditions and traumas.

In another aspect, some embodiments of the disclosure relate to a method of treating cells ex vivo or in vitro. The method includes adding an effective amount of any of the modified heat shock protein or fragment thereof disclosed herein to a culture media. In some embodiments, the method maintains the viability of the cells during their culture and propagation.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claim.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows multiple sequence alignments for members of the HSP70 family of proteins across species. Alignments were done using the Clustalχ algorithms available at the EMBL-EBI website (www.ebi.ac.uk/Tools/msa/clustalo/). Each sequence was obtained from the Uniprot protein database (www.uniprot.org/) with the unique Uniprot accession number for each protein specified in parentheses. The current HSP70 gene name for each sequence is followed, in some cases, by other common names used in the scientific literature. Sequences are shown for mammals: Homo sapiens (Human HSP72), Mus musculus (Mouse HSP72), and Oryctolagus cuniculus (Rabbit GRP75 and BiP). HSP70 sequences are also shown for four types of flatworms: Neobenedenia melleni, Dugesi japonica, Echinococcus multilocularis (Fox Tapeworm), and Echinococcus granulosus (Dog Tapeworm). And HSP70 sequences are shown for two plants: Zea mays (Maize) and Solanum lycopersicum (Tomato). Sites modified to eliminate glycosylation are highlighted in yellow. The site modified to remove acid-labile (e.g. aspartyl-proline) cleavage is highlighted in green. Sites modified to eliminate alkaline-labile (e.g. asparagine-proline) cleavage are highlighted in light blue.

FIG. 2 shows multiple sequence alignments for members of the human HSP70 family of proteins. Alignments were done using the ClustalΩ algorithms available at the EMBL-EBI website (www.ebi.ac.uk/Tools/msa/clustalo/). Each sequence was obtained from the Uniprot protein database (www.uniprot.org/) with the unique Uniprot accession number for each protein specified in parentheses. The current HSP70 gene name for each sequence is followed, in some cases, by other common names used in the scientific literature. Sequence of the HSP72 protein (also known as the HSPA1A gene) is in red. Sequence of the BiP protein (also known as GRP78 and the HSPAS gene) is in blue. Those residues underlined and in bold are putative N-glycosylation sites.

FIGS. 3A and 3B show SDS-polyacrylamide gel electrophoresis (PAGE) of wild-type HSP72 and HSP72 fused to a single chain Fv fragment of 3E10 (“Fv-HSP72”), for transport across the cell membrane, before (G) and after ( ) enzymatic deglycosylation with 5 mU of PNGase F (Prozyme) in a phosphate reaction buffer. The shift in mobility for the two types of proteins indicates removal of glycosylation sites. A) Note the mobility does not change for the 3E10 scFv fragment alone when it too is deglycosylated. So the glycosylation of the Fv-HSP72 fusion is relegated to the HSP72 portion of the molecule. Schematics of each protein are included in the inset: 1) the Fv-HSP72 published in the literature consists of the murine 3E10 scFv fused to a c-Myc tag followed by a histidine (His) tag and the human HSP72; 2) the HSP72 alone carries a His tag; and 3) the 3E10 scFv fragment alone carries both c-Myc and His tags. Glycosylation of molecules in the schematics are represented by (ψ ψ). B) The proteins were transferred on to a nitrocellulose membrane and their identity were verified by probing with an anti-HSP72 antibody (clone C92, StressMarq). Molecular weight markers (M) run in the far-left column show a range of proteins resolved on the gel from 20-250 kD.

FIGS. 4A and 4B show a pairwise alignment of the human and mouse HSP72 proteins. Alignments were conducted according to the Needleman-Wunsch algorithm using ClustalW at the EMBL-EBI website (www.ebi.ac.uk/Tools/psa/). Each sequence was obtained from the Uniprot protein database (www.uniprot.org/) with the unique Uniprot accession number for each protein specified in parentheses. Sequences for the human and mouse HSP72 proteins are in red and blue, respectively. A) Those residues underlined and in bold are putative N-glycosylation sites. Each site was given a numerical designation between 1 and 5. B) Those residues underlined and in bold are putative acidic or alkaline cleavage sites. One acid-labile cleavage site contains an aspartic acid (D)—proline (P) linkage and is designated as the D-P site. The D-P site is only present in human HSP72, but not in mouse HSP72. There are 3 asparagine (N)—proline (P) sites to be completely cleaved under strong alkaline conditions: Sites I, III and VII. Site VII is only present in human HSP72, not mouse HSP72. There are 4 sites that may be partially cleaved under strong alkaline conditions. These sites contain either an asparagine (N)—leucine (L) linkage (Site V), an asparagine (N)—serine (S) linkage (Site IV), an asparagine (N)—threonine (T) linkage (Sites II and VI). A|(line) indicates positions which have identical amino acids. A “:” (colon) indicates conservation of amino acids with strongly similar properties; e.g., properties which score >0.5 in the Gonnet PAM 250 matrix. A “.” (period) indicates amino acids with weakly similar properties; e.g., properties which score≤0.5 in the Gonnet PAM 250 matrix. Further description of the PAM 250 matrix can be found in, for example, W. A Pearson, (1990) Rapid and Sensitive Sequence Comparison with FASTP and FASTA, in Methods in Enzymology, ed. R. Doolittle (ISBN 0-12-182084-X, Academic Press, San Diego) 183:63-98, which is incorporated by reference in its entirety.

FIGS. 5A-5C show pairwise alignments of the human wild-type and modified HSP72 proteins. Alignments were conducted according to the Needleman-Wunsch algorithm using ClustalW at the EMBL-EBI website (www.ebi.ac.uk/Tools/psa/). The wild-type human HSP72 sequence was obtained from the Uniprot protein database (Uniprot accession number PODMV8). Sites 1-5 that are underlined and in bold are putative N-glycosylation sites. Sites I-VII that are underlined and in bold are putative alkaline-labile sites. The acid labile bond between residues D80 and P81 is underlined, in bold, and designated as the D-P site. FIG. 5A depicts a scenario where all sites are modified to eliminate N-glycosylation, alkaline-labile cleavage sites and acid-labile cleavage sites. FIG. 5B depicts a scenario where all N-glycosylation sites and the acid-labile cleavage site are modified. FIG. 5C depicts a scenario where all N-glycosylation sites, the acid-labile cleavage site and one alkaline-labile cleavage site is modified. A “|” (line) indicates positions which have identical amino acids. A “:” (colon) indicates conservation of amino acids with strongly similar properties; properties which score >0.5 in the Gonnet PAM 250 matrix. A “.” (period) indicates amino acids with weakly similar properties; properties which score≤0.5 in the Gonnet PAM 250 matrix.

FIG. 6 shows examples of multiple sequence alignments and pairwise sequence alignments for members of the HSP40, HSP60, HSP90, HSP105/110 and HSP27 families of proteins. N-glycosylation, acid-labile (D-P), and alkaline-labile (N-P, N-L, N-S and N-T) sites are in bold and underlined. N-glycosylation sites are further differentiated from the cleavage sites by highlighting in yellow. Multiple sequence alignments were done using the Clustalψ algorithms available at the EMBL-EBI web site (www.ebi.ac.uk/Tools/msa/clustalo/). Alignments were conducted according to the Needleman-Wunsch algorithm using ClustalW at the EMBL-EBI website (www.ebi.ac.uk/Tools/psa/). Each sequence was obtained from the Uniprot protein database (www.uniprot.org/) with the unique Uniprot accession number for each protein specified in parentheses. A “|” (line) in the pairwise alignments or an * (asterisk) in the multiple alignments indicates positions which have identical amino acids. A “:” (colon) indicates conservation of amino acids with strongly similar properties; properties which score >0.5 in the Gonnet PAM 250 matrix. A “.” (period) indicates amino acids with weakly similar properties; properties which score≤0.5 in the PAM 250 matrix.

FIG. 7 shows schematic representations of four Fv-HSP72 molecules (RBB001, RBB002, RBB003 and RBB004). RBB001 is the fusion of a 3E10 scFv fragment fused to a c-Myc tag, a His tag and the wild-type human HSP72, in the N- to C-terminal direction. RBB001 was produced in Pichia fungal cells and retained all of the wild-type glycosylation sites. RBB001 was used in early studies of Fv-HSP72 in stroke and myocardial infarction studies. RBB002 has the His tag relocated to the N-terminus, followed by a 3E10 scFv fragment fused to a linker and the wild-type human HSP72. RBB002 retains all of the wild-type glycosylation sites but has the D-P site mutated to D-A. Mutation of the five HSP72 N-glycosylation sites and the D-P site as described herein resulted in the creation of RBB003. RBB004 is identical to RBB003 except for the removal of the His tag from the protein sequence. Symbol ψ represents a carbohydrate structure attached to the asparagine at the N-X-S/T/C site.

FIGS. 8A and 8B show SDS-PAGE gels (4-20% gradient) of RBB001, RBB002 and RBB003 produced in fungal cells (A) and mammalian cells (B). All materials affinity purified via His-tag binding to a nickel agarose resin and elution by an imidazole gradient. A) Production in Pichia fungal cells. The product from three separate transfected cell lines is shown for RBB002 and RBB003 alongside the first generation RBB001 product and HSP72 alone. Faster migration of the non-glycosylated RBB003 compared to RBB001 and RBB002 was noted. Protein bands were visualized under ultraviolet (UV) light with a tri-halo compound that reacts with tryptophan. B) Production in CHO mammalian cells. Multiple fractions from a single purification run for RBB002 and RBB003 are shown. Load: CHO cell supernatant loaded on to a nickel agarose resin column. FT: Flow thru from the nickel agarose column. Fr1-3: Fraction eluted from the nickel agarose column using an imidazole gradient. Faster migration of the non-glycosylated RBB003 compared to the glycosylated RBB002 was noted. Proteins were visualized by Coomassie staining. Molecular weight markers (M) run in the far-right column show a range of proteins resolved on the gels from 30-150 kD.

FIGS. 9A and 9B show RBB003 production in the baculovirus insect system and in two E. coli bacterial strains. A) Production of RBB003 in insect cells was achieved by administration of a viral load to a high concentration of Cabbage Looper Ovary cells (High Five cells from Invitrogen) which were harvested and lysed at 24, 48 and 72 hours after infection.

RBB003 was affinity purified via His-tag binding to a cobalt resin and elution by an imidazole gradient. Protein bands were resolved by a 4-20% SDS-PAGE gel and visualized by Coomassie staining. B) Production of RBB003 in two E. coli bacterial strains was achieved by transfection of the strains with codon-optimized versions of the RBB003 gene (clones 6 and 9). Protein was extracted from the cell pellet (P) and the periplasmic supernatant (SN) and resolved with a 4-20% SDS-PAGE gel before transfer to a nitrocellulose membrane for western blotting. RBB003 was visualized with an anti-HSP72 antibody (clone C92, StressMarq). A sample of RBB003 produced in CHO cells was run as a positive control. Molecular weight markers (M) were run in the second column from the right and show a range of proteins resolved on the gel from 29-124 kD.

FIGS. 10A-10C illustrates an in vitro cell-based assay performed to measure cell death. A) Cell-Tox Green is a fluorescent, cell-impermeant DNA binding dye that was added to media during tissue culture incubations. For In Vitro Study 1, human primary cardiomyocytes were grown in a 96-well plate and intoxicated with hydrogen peroxide (H₂O₂) to induce oxidative stress and subsequent apoptosis, resulting in increased exposure of DNA and concomitant increases in fluorescent signal. CellTox Green excitation occurs at 490 nanometers (nm) and emission is monitored at 525 nm with a cut-off of 515 nm in a plate reader. Data points represent the average of 4 wells. Error bars represent standard error of the mean. B) For In Vitro Study 2, human primary cardiomyocytes were exposed to 2.6 mM H₂O₂ at time T=0 hours and throughout the course of the study. A set of control cells were not intoxicated (“No H₂O₂”; brown circle). Those cells receiving “H₂O₂ Only” (red square) served as a positive control. Treatment of cells, 30 minutes after the start of intoxication, with 3E10-Fv alone (green diamond) or Hsp72 alone (purple cross) did not affect the increase in cell death. However, treatment with Fv-HSP72 (dark blue triangle) at 30 minutes after intoxication significantly attenuated the course of apoptosis (p=0.0006). Fv-HSP72 entry through the ENT2 channel is confirmed by competitive inhibition using a 1,000-fold molar excess of 3E10-Fv added prior to Fv-HSP72 treatment (light blue triangle). Despite the inhibition of Fv-HSP72 efficacy, the addition of 3E10-Fv still resulted in a significant attenuation of apoptosis at T=12 hours (p=0.002). Number of wells receiving each treatment are indicated in parentheses ( ). Percentage values reflect normalization of the average fluorescent signal at the last reading divided by the maximum signal obtained after total cell lysis in three of the No H₂O₂ control wells. Statistical significance determined by Student's t-test. * signifies statistically significant; *** highly significant. Error bars represent standard error of the mean. Cardiomyocytes kept in a 5% CO₂ and 37° C. incubator between readings. C) A schematic summary of the results of In Vitro Study 2.

FIG. 11 illustrates an in vitro cell-based assay performed to measure oxidative stress. For In Vitro Study 3, wells selected from the cell death studies described in FIG. 10 above were incubated with a lysis buffer and SDS extraction buffer. The protein extracts were incubated with DNPH (DNP Reaction) to derivatize any carbonyl adducts formed on proteins due to oxidation. A control solution sans DNPH was run in parallel (Control Reaction). Greater amounts of carbonylation were seen in cells exposed only to H₂O₂ (red square) compared to No H₂O₂ (brown circle). However, treatment with 0.05 nmole RBB001 (orange triangle) at 30 minutes after intoxication kept protein carbonylation levels comparable to those cells never exposed to H₂O₂, and in this particular example, for up to 17 hours af ter intoxication. In fact, the difference in carbonylation between H₂O₂ cells left untreated and those treated with RBB001 is statistically significant (p=0.011). The Control Reactions clearly showed that the anti-DNP did not cross-react with total protein extracts unless the carbonyls are derivatized. Absorbances obtained from control wells are graphed in a lighter color than their DNP reaction counterparts. Each reaction was run in triplicate. Average represented by a horizontal line (-). TMB absorbances were read at 450 nm in a plate reader. * signifies statistically significant.

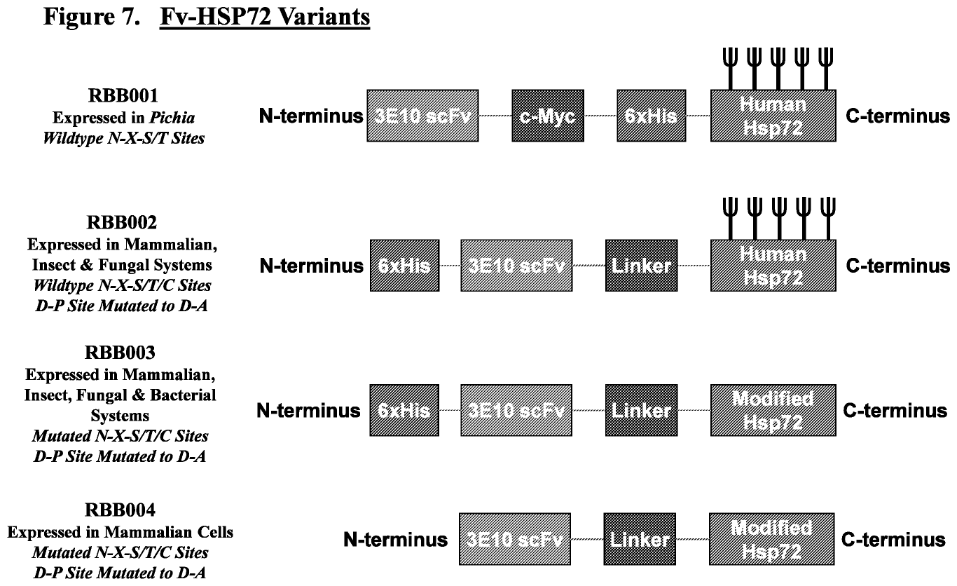

FIGS. 12A to 12D graphically summarize the results from in vitro efficacy testing in cardiomyocytes of the three Fv-HSP72 variants described in FIG. 7. A) For In Vitro Study 4, human primary cardiomyocytes were exposed to 1.5 mM H₂O₂ at time T=0 hours. Fluorescence measurements of CellTox Green as described in FIG. 10 are shown for the last four readings from a 26 hour study. A set of control cells were not intoxicated (“No H₂O₂” brown circle). Those cells receiving H₂O₂ Only (red square) served as a positive control. Treatment of cells, 30 minutes after the start of intoxication, with 0.05 nmoles of RBB001 (blue triangle), RBB002 (green diamond), and RBB003 from mammalian CHO cells (pink circle) or E. coli (black circle), inhibited apoptosis to varying degrees compared to those cells receiving H₂O₂ Only. Data points represent the average of 7 wells. Error bars represent the standard error of the mean. Seven of the wells were subsequently exposed to lysis buffer and the signal generated from total lysis of the cells was used to calculate the percentage values in C. B) Student's t-test analysis of each Fv-HSP72's ability to inhibit cell death compared to the H₂O₂ Only control. * signifies statistically significant; * * * highly significant. C) Conversion of the random fluorescence units to percentage of total cell death entailed normalizing the readings with the average fluorescence value obtained after total cell lysis of 7 wells. The non-glycosylated RBB003s proved more efficacious than RBB002 containing the wild-type HSP72 sequence. D) Carbonylation analysis for testing oxidative stress. Student's t-test of No H₂O₂ and the Fv-HSP72 constructs versus the H₂O₂ Only control. RBB003 constructs of Fv-HSP72 proved to lower oxidative stress in cardiomyocytes to levels below that even seen for the No H₂O₂ controls. ** signifies statistically significant. Each reaction was run in triplicate. Average represented by a horizontal line (-). Absorbances obtained from control wells are graphed in a lighter color than their DNP reaction counterparts. TMB absorbances were read at 450 nm in a plate reader

FIG. 13 summarizes the results from In Vitro Study 5, which was performed to evaluate the cellular uptake of RBB001 and RBB004 in A549 cells over a period of 4 hours. The cells reached confluency prior to the addition of either Fv-HSP72 molecule. This study investigated cellular uptake without H₂O₂ intoxication, instead of relying on the natural course of cell death during confluency to provide sufficient extracellular DNA for internalization of either Fv-HSP72. Cellular internalization of RBB001 and RBB004 was measured at 30 minutes (orange triangle; brown square), 1 hour (blue triangle; blue square) and 4 hours (green triangle; green square) after treatment. At each timepoint, culture media was aspirated from selected wells, rinsed with PBS, and the cells were fixed in 100% ice cold ethanol. Cells were then probed with biotinylated Protein L to detect the 3E10 scFv domain followed by the addition of Streptavidin conjugated to the AlexaFluor594 (Strep-AF594) fluorochrome. Strep-AF594 excitation occurs at 590 nm and emission is monitored at 617 nm with a cut-off of 610 nm in a plate reader. The cells were then reprobed with a DNA stain such as 4′,6-Diamidine-2′-phenylindole dihydrochloride (DAPI) or Hoechst 33342 (Bisbenzimide) in order to normalize the Strep-AF594 signal with a measure of the number of nuclei, hence the number of cells, in each well. The DAPI stain is excited in the plate reader at 358 nm and the emission is monitored at 461 nm with a cut-off of 455 nm. Each time point was run in triplicate. Averages are represented by a horizontal line (-) for each data set. Measured values at each of the time points were compared to those in control cells not receiving Fv-HSP72. These control cells were probed either with biotinylated Protein L and Strep-AF594 (red circle) or Strep-AF594 alone (brown circle) to determine the background signal due to cross-reactivity with other cellular proteins and structures. After 4 hours, RBB001 internalization, on average, was greater than the background signal obtained with the biotinylated Protein L/Strep-AF594 control, but is not statistically significant (p=0.1353). On the other hand, RBB004 internalization was statistically significant within 30 minutes (p=0.0029) and even at 4 hours (p=0.0099). ** signifies statistically significant

FIGS. 14A-14C summarize the results from in vitro efficacy testing and cellular uptake of RBB004. A) For In Vitro Study 6, human primary cardiomyocytes were intoxicated with 1.5 mM H₂O₂ at time T=0 hours. Fluorescence measurements of CellTox Green as described in FIG. 10 are shown for the last three readings from a 17 hour study. A set of control cells were not intoxicated (“No H₂O₂” brown circle). Those cells receiving H₂O₂ Only (red square) served as a positive control. Treatment of cells, 30 minutes after the start of intoxication, with 0.05 nmoles of RBB004 (blue triangle) inhibited apoptosis compared to those cells receiving H₂O₂ Only (p=0.0005). Data points represent the average of 7 wells. Error bars represent the standard error of the mean. Twelve wells were subsequently exposed to lysis buffer and the signals generated from total lysis of the cells was used to normalize the values plotted in the graph. The total lysis signals were also used to calculate the percentage values. These percentage values represent the average amount of cell death in the wells compared to the total amount of cells present in the wells. B) Three of the wells from each group were also subjected to oxidative stress analysis as described in FIG. 11. While the difference in protein carbonylation between H₂O₂ cells left untreated and those treated with RBB004 is clearly seen in the graph, a Student's t-test finds the difference not quite statistically significant (p=0.0834). C) Two sets of cardiomyocytes were intoxicated with 3 mM H₂O₂ at time T=0 hours to evaluate cellular uptake as described in FIG. 13. Seven wells each were treated with fresh production lots of RBB001 (brown square) and RBB004 (blue square) and 3 wells were neither intoxicated with 14202 nor treated with Fv-HSP72 (brown circle). Cellular internalization was measured by adding the Fv-HSP72s 30 minutes after intoxication and then fixing all cells in 100% ice cold ethanol 4 hours later. Unlike the study in FIG. 13, this study used H₂O₂ intoxication to generate cell death and extracellular DNA. The study also determined if the Protein L/Strep-AF594 probing of the cells is detecting internalized Fv-HSP72s versus those molecules trapped on the surface of the cardiomyocytes. Five of the 7 wells treated with each Fv-HSP72 was briefly incubated with trypsin to proteolytically digest or “shave off” any RBB001 or RBB004 attached to the extracellular surface of the cardiomyocytes. Distribution of signal from shaved cells (n=5) was not significantly different from unshaved cells (n=2) suggesting all signals obtained are from internalized Fv-HSP72s. RBB004 internalization into the human primary cardiomyocytes is far greater than RBB001 at 4 hours after treatment and statistically significant (p=0.0007). Averages are represented by a horizontal line (-) for each data set. *** signifies a highly statistically significant result.

FIGS. 15A to 15E summarize the results from in vitro testing of the efficacy of RBB001, RBB002 and RBB003 in a cell-based assay using pulmonary cells. A) For In Vitro Study 7, human primary alveolar cells were exposed to 1.5 mM H₂O₂ at time T=0 hours. Fluorescence measurements of CellTox Green as described in FIG. 10 are shown for the last five readings from a 14 hour study. A set of control cells were not intoxicated (“No H₂O₂” brown circle). Those cells receiving H₂O₂ Only (red square) served as a positive control. Treatment of cells, 30 minutes after the start of intoxication, with 0.05 nmoles of RBB001 (blue triangle), RBB002 (purple diamond), and RBB003 from mammalian CHO cells (orange circle) or E. coli (black circle), inhibited apoptosis to varying degrees compared to those cells receiving H₂O₂ Only. Data points represent the average of 7 semi-permeable membrane inserts for all time points except at 14 hours when only 4 inserts were used and the other 3 were subjected to barrier integrity analysis. Error bars represent the standard error of the mean. Seven of the remaining wells were subsequently exposed to lysis buffer and the signal generated from total lysis of the cells was used to calculate the percentage values in C. B) Student's t-test analysis of each Fv-HSP72's ability to inhibit cell death compared to the H₂O₂ Only control. All constructs had p values>0.05. C) Conversion of the random fluorescence units to percentage of total cell death entailed normalizing the readings with the average fluorescence value obtained after total cell lysis of 7 wells. The non-glycosylated RBB003s appear more efficacious than RBB002 containing the wild-type HSP72 sequence. D) Barrier integrity was determined by adding the fluorescent protein R- Phycoerythrin, in media, to 3 of the inserts containing the cells and tracking the migration of the R-PE across the alveolar monolayer and the semi-permeable membrane into the media in the wells below. E) Cell not exposed to H₂O₂ had less fluorescent protein migration into the wells compared to those cells exposed to H₂O_{2.} Cells treated with Fv-Hsp72s had an intermediate amount of R-PE migration; those cells receiving RBB003 showing, on average, less of a barrier disruption.

FIGS. 16A and 16B summarize the results from in vivo evaluation of Fv-HSP72 constructs in a toxic inhalation model. A) A Kaplan-Meier survival curve is shown for rats exposed to phosgene vapor for 10 minutes, then intravenously treated either with RBB001 or RBB003 at 30 minutes after the exposure had ended. There were 9 rats in each group at the start of the study. No rats injected with 174 nmoles/kg (20 mg/kg) RBB001 survived the 25 hour study (blue trace). Of the rats injected with 174 nmoles/kg of RBB003, three survived until study termination (33.3%, red trace). Rats receiving 87 nmoles/kg (10 mg/kg) of RBB003 had only 1 survivor (11.1%, green trace). B) Rats treated with RBB003 had statistically significant reductions in oxidative stress levels in their lung tissues compared to animals that were left untreated, as determined by Student's t-test of the 9 Phosgene Only and 12 RBB003 absorbance values plotted in the graph (p=0.0448). Oxidation was determined by SDS extraction of proteins from lung tissues and probing for carbonyl adducts using DNPH and anti-DNP antibodies as described in the text and illustrated in FIG. 11. Each reaction was run in triplicate. Average represented by a horizontal line (-). Absorbances obtained from control wells are graphed in a lighter color than their DNP reaction counterparts. TMB absorbances were read at 450 nm in a plate reader.

FIGS. 17A and 17B depict amino acid sequences of Fv-HSP72 molecules, RBB002, and RBB003 that are generated using human HSP proteins.

FIGS. 18A and 18B show nucleotide sequences encoding RBB002 and RBB003 that are codon-optimized for Pichia pastoris.

FIGS. 19A and 19B show nucleotide sequences encoding RBB002 and RBB003 that are codon-optimized for baculovirus.

FIGS. 20A and 20B show nucleotide sequences encoding RBB002 and RBB003 that are codon-optimized for Escherichia coli. Amino acid sequences of the codon-optimized RBB002 and RBB003 are also presented.

FIGS. 21A-21C show nucleotide sequences encoding RBB002, RBB003 and RBB004 that are codon-optimized for mammal. Amino acid sequences of the codon-optimized RBB002, RBB003 and RBB004 are also presented.

### DETAILED DESCRIPTION

The present disclosure relates generally to the field of molecular biology and medicine, including compositions, systems, and methods for producing modified heat shock proteins (HSPs), the modified HSPs produced by the methods, and use thereof. In one aspect, the present disclosure provides that elimination of the putative glycosylation sites in human HSP72 by modifying the amino acid sequences at those sites creates a modified HSP that is non-glycosylated yet still retains its functionality. This is a surprising result given that extensive glycosylation of HSP72 with oligomannose in the yeast Pichia pastoris produces an efficacious product, yet a change in glycosylation upon production in a mammalian cell line produces a loss of activity. Some embodiments of the disclosure relate to a modified human HSP72 sequence, different from the wild-type sequence, which can be produced via secretion from mammalian, insect, fungal and even bacterial cells without loss of activity.

In one aspect, some embodiments of the disclosure relate to HSPs that have one or more amino acid residues replaced with ones that disrupt glycosylation. The disclosure also provides, in some embodiments, HSPs comprising one or more amino acid residues that are replaced with ones that are not cleaved under a low pH or high pH.

In one aspect, some embodiments of the disclosure relate to polypeptide sequences for use in a method of medical treatment. Such polypeptide sequences can be obtained by modifying HSPs including, but not limited to, any members of chaperonins, HSP27, HSP40, HSP60, HSP70, HSP72, HSP90, HSP105/110, and the “small heat shock proteins” family of proteins.

In one aspect, the disclosure provides evidence of changes in efficacy, specifically for HSP72, when the source of its production, and hence its glycosylation pattern, is changed. The disclosure further provides evidence of improved efficacy when the putative glycosylation sites are removed altogether and replaced with conserved amino acids.

In one aspect, the disclosure also describes the linkage of the modified HSPs to antibodies, antibody fragments or other protein structures that target nucleic acids, histones and other intracellular molecules that can be transported from the extracellular environment into a cell through either energy-dependent or energy-independent processes (e.g. channels, transporters, receptors, etc.).

In one aspect, some embodiments of the disclosure relate to the use of modified heat shock proteins to treat ocular, neurological, muscular, hepatic, renal, integumentary, cardiovascular and pulmonary conditions and traumas.

In one aspect, some embodiments of the disclosure relate to a method of treating cells ex vivo or in vitro. In some embodiments, the method maintains the viability of the cells during their culture and propagation.

### Definitions

Unless defined otherwise, all technical and scientific terms used herein have the same meaning as is commonly understood by one of skill in the art to which the claimed subject matter belongs. It is to be understood that the detailed descriptions are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with those embodiments is included in at least some embodiments, but not necessarily all embodiments, of the disclosure.

“About” has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “polypeptide”, “peptide”, and “protein” are used interchangeably herein to designate a linear series of amino acid residues connected one to the other by peptide bonds, which includes proteins, polypeptides, oligopeptides, peptides, and fragments thereof. The protein may be made up of naturally occurring amino acids and/or synthetic (e.g. modified or non-naturally occurring) amino acids. Thus “amino acid”, or “peptide residue”, as used herein means both naturally occurring and synthetic amino acids. The terms “polypeptide”, “peptide”, and “protein” include fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a cellular transporter, e.g. 3E10 antibody or fragment thereof, a fluorescent protein, β-galactosidase, luciferase, etc. and the like. Furthermore, it should be noted that a dash at the beginning or end of an amino acid sequence indicates either a peptide bond to a further sequence of one or more amino acid residues or a covalent bond to a carboxyl or hydroxyl end group. However, the absence of a dash should not be construed to mean that such peptide bond or covalent bond to a carboxyl or hydroxyl end group is not present, as it is conventional in representation of amino acid sequences to omit such.

As used herein, “primary sequence” refers to the amino acid sequence of a protein. The primary sequence of an amino acid may include a naturally occurring amino acid sequence of the protein or synthetic sequence.

In the context of a polypeptide sequence, the terms “residue” and “amino acid” are used interchangeably herein.

As used herein, “kD” refers to a molecular weight of 1 kiloDalton or 1000 Daltons.

As used herein, “oligosaccharide” or “moieties” is used interchangeably with the word “carbohydrate”.

As used herein, a “heat shock protein” or “HSP” is a protein molecule that has been categorized as a member of the chaperonins, HSP27, HSP40, HSP60, HSP70, HSP90, HSP105/110, the “small heat shock proteins” or other newly emerging family of heat shock proteins in the scientific literature by sequence comparison to homologous, orthologous or paralogous family members. A divergent member can also be identified as a heat shock protein by analysis of its induction during cellular stress or its activities to ameliorate that stress.

As used herein, “post-translationally modified” or a “post-translational modification” includes, but is not limited to, phosphorylation, acetylation, methylation, ubiquitination, sumoylation, hydroxylation, citrullination (deimination), deamidation, oxidation, reduction, or glycosylation. The modification can occur either through biological processes (such as enzymatically in vivo or in vitro) or through synthetic processes (such as a chemical reaction in vitro).

As used herein, “glycosylation” refers to a reaction in which a carbohydrate, e.g. a glycosyl donor, is attached to a hydroxyl or other functional group of another molecule (a glycosyl acceptor). Glycosylation can include several types such as N-linked glycosylation, O-linked glycosylation, phosphor-serine glycosylation, C-mannosylation and glypiation.

The term “N-glycosylation” or “N-linked glycosylation” refers to, in the context of glycosylation on a peptide or protein sequence, the attachment of the sugar molecule oligosaccharide known as glycan to a nitrogen atom (e.g., amide nitrogen of asparagine (Asn) residue of a protein). The N-linked glycosylation process occurs in eukaryotes as well as other classes such as archaea. Different species may synthesize different types of N-linked glycan that is attached to the protein. For example, N-glycosylation can occur during protein secretion from eukaryotic cells at the N-X-S/T/C site whether those cells are fungal, insect or mammalian. The N-glycosylation site of any given protein can be a putative site, e.g. an amino acid sequence that is deemed to be glycosylated in view of the sequence similarity to a known N-glycosylation sequence. The N-glycosylation site can also be a site where the glycosylation on that site was experimentally determined. Assays and methodologies suitable for measuring N-glycosylation site occupancy in a glycoprotein are known in the art.

As used herein, “acid-labile cleavage” refers to, in the context of peptide cleavage, a cleavage, which may be interchangeably used with cut or breakdown, of a peptide or protein sequence when the sequence is subjected to an acidic environment. For example, a peptide can have one or more amino acid sequences that can be cleaved or cut when it is exposed to a low pH such as, e.g. pH of less than or equal to 7. The acid-labile cleavage site of the disclosure can generally be any acid-labile cleavage sites known in the art. In some embodiments, the acid-labile cleavage site comprises aspartic acid-proline (D-P) residues with cleavage occurring between the two residues. In some embodiments, the acid-labile site can be a putative site, e.g. an amino acid sequence that is deemed to be cleaved or cut under a low pH in view of the sequence similarity to a known acid-labile sequence. In some embodiments, the acid-labile cleavage site can also be a site where the acid-labile cleavage on that site was experimentally determined. As used herein, “alkaline-labile cleavage” refers to, in the context of peptide cleavage, a cleavage, which may be interchangeably used with cut or breakdown, of a peptide or protein sequence when the sequence is subjected to an alkaline environment. For example, a peptide can have one or more amino acid sequences that can be cleaved or cut when it is exposed to a high pH such as, e.g. pH of higher than or equal to 7. In some embodiments, the alkaline-labile cleavage site can have asparagine-proline (N-P) residues with cleavage occurring between the two residues. The N-P linkage is completely cleaved under alkaline conditions. In some embodiments, the alkaline-labile cleavage sites comprises asparagine-leucine (N-L), asparagine-serine (N-S), or asparagine-threonine (N-T) residues with cleavage occurring between the two residues. N-L, N-S and N-T linkages are partially cleaved under alkaline conditions. The alkaline-labile site of any given protein can be a putative site, e.g. an amino acid sequence that is deemed to be cleaved or cut under a high pH in view of the sequence similarity to a known alkaline-labile sequence. The alkaline-labile cleavage site can also be a site where the alkaline-labile cleavage on that site was experimentally determined.

As used herein, a “modified heat shock protein” or “modified HSP” is a protein molecule that has been created by genetically changing one or more amino acids in the protein sequence of HSP. In some cases, the modified HSP includes a sequence of HSP that was modified from the wild-type sequence to remove one or more glycosylation sites and/or one or more acid-labile cleavage sites and/or one or more alkaline-labile cleavage sites from the wild-type sequence.

As used herein, “non-conservative amino acid substitution,” in particular in the context of modification of HSPs to modify one or more glycosylation sites and/or one or more acid-labile cleavage sites and/or one or more alkaline-labile cleavage sites, includes modification, substitution, deletion and/or addition of one or more amino acids from the primary sequence of HSP, leading to the formation of a peptide that has substantial reduction or abolition of glycosylation and/or acid-labile cleavage and/or alkaline-labile cleavage. The term “substantial reduction” or “substantially reduced” refer to, in the context of glycosylation and/or acid-labile cleavage and/or alkaline labile cleavage of HSPs, at least about 10% to about 100% reduction or abolition, compared to the level of glycosylation and/or acid-labile cleavage and/or alkaline-labile cleavage of the primary HSP without the modification. In some embodiments, the modification on the glycosylation site(s) and/or acid-labile cleavage site(s) and/or alkaline-labile cleavage site(s) does not substantially affect other activities of the HSPs (e.g. the ability to inhibit cell death) In some embodiments, the modified HSP retain at least 10% to 100% of such activities of the corresponding unmodified HSP sequence, e.g., an primary HSP without the modification.

The terms “derivative” and “variant” refer to without limitation any compound, e.g. protein which has a structure or sequence derived from the proteins of the present disclosure and whose structure/sequence is sufficiently similar to those disclosed herein and based upon that similarity, would be expected, by one skilled in the art, to exhibit the same or similar activities and utilities as the claimed and/or referenced protein, thereby also interchangeably referred to “functional equivalent”. Modifications to obtain “derivative” or “variant” includes, for example, by addition, deletion and/or substitution of one or more of the amino acid residues. In some embodiments of the disclosure, the functional equivalent or fragment of the functional equivalent has one or more conservative amino acid substitutions. The term “conservative amino acid substitution” refers to substitution of an amino acid to another amino acid that has similar properties to the original amino acid. The groups of conservative amino acids are shown in TABLE 1 below:

| Group | Name of the amino acids |
|---|---|
| Aliphatic | Gly (G), Ala (A), Val (V), Leu (L), |
| | Ile (I) |
| Hydroxyl or Sulfur/ | Ser (S), Cys (C), Thr (T), Met (M) |
| Selenium-containing | |
| Cyclic | Pro (P) |
| Aromatic | Phe (F), Tyr (Y), Trp (W) |
| Basic | His (H), Lys (K), Arg (R) |
| Acidic and their Amide | Asp (D), Glu (E), Asn (N), Gln (Q) |

Conservative substitutions may be introduced in any position of a preferred predetermined peptide or fragment thereof.

“Percentage of sequence identity” is determined by comparing two optimally aligned sequences over a comparison window, wherein the portion of the polynucleotide or polypeptide sequence in the comparison window may include additions or deletions (e.g., gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acids or polypeptide sequences, refer to two or more sequences or subsequences that are the same or have a specified percentage of amino acid residues or nucleotides that are the same (e.g., 60% identity, optionally 65%, 70%, 75%, 80%, 85%, 90%, 95%, 98%, or 99% identity over a specified region, e.g., of the entire polypeptide sequences or individual domains of the polypeptides), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 5 to 50 nucleotides or polypeptide sequences in length, or more preferably over a region that is 100 to 500 or 1000 or more nucleotides or polypeptide sequences in length. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov).

As used herein, “nucleic acids” will include nucleosides, nucleotides, oligonucleotides and fragments of DNA or RNA of any length that is capable of being opsonized or transported across the plasma membrane. Nucleosides and nucleotides will include, but are not limited to, ribonucleosides, ribonucleotides, deoxyribonucleosides and deoxyribonucleotides. Nucleosides and nucleotides will include, but are not limited to, ones containing purine or pyrimidine bases of adenine, guanine, cytosine, thymine and uracil. As used herein, peptide nucleic acids (PNAs) with the bases attached to a backbone composed of repeating N-(2-aminoethyl)-glycine units linked by peptide bonds, will also be considered nucleic acids.

As used herein, “codon” refers to a sequence of three nucleotides that together form a unit of genetic code in a DNA or RNA molecule. As used herein the term “codon degeneracy” refers to the nature in the genetic code permitting variation of the nucleotide sequence without affecting the amino acid sequence of an encoded polypeptide.

The term “codon-optimized” or “codon optimization” refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Given the large number of gene sequences available for a wide variety of animal, plant and microbial species, it is possible to calculate the relative frequencies of codon usage. Codon usage tables are readily available, for example, at the “Codon Usage Database” available at www.kazusa.or.jp/codon/(visited Mar. 20, 2008). By utilizing the knowledge on codon usage or codon preference in each organism, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various methods known to those skilled in the art.

The term “isolated” is intended to mean that a compound is separated from all or some of the components that accompany it in nature. “Isolated” also refers to the state of a compound separated from all or some of the components that accompany it during manufacture (e.g., chemical synthesis, recombinant expression, culture medium, and the like).

As used herein, a “conjugate” is a molecule having at least two parts associated such that the parts of the molecule remain associated if transported to a target. Conjugates include fusion proteins linked to each other via their polypeptide structure, through genetic expression of a DNA molecule encoding the fusion protein. Conjugates can also include coupled proteins in which pre-formed sequences are associated by cross-linking agents or associations, such as aggregates of the parts of the molecule. Conjugation can also mean the cross-linking of a molecule for transportation coupled to the lipid components of a liposome to transport heat shock proteins (HSPs) contained within.

As used herein, “transport” across the plasma membrane includes any energy-dependent or independent process that translocates or transfects a molecule from the extracellular environment to the intracellular environment. Such processes include, but are not limited to, the conjugation of proteins, protein fragments, peptide leader sequences and poly-cation sequences that target nucleic acids. They also include the use of specific cell-penetrating proteins and peptides, such as phospholipid binding proteins (e.g. annexins), histones, TAT proteins from the HIV virus or Antennapedia proteins from insects, to name just a few. They also include direct attachment, either by genetic means or by chemical conjugation, of nucleic acids to the HSPs.

They also include binding of ions transported across the membrane, such as binding of calcium and calcium ions transported through channels.

As used herein, a “channel” or “transporter” can include, but are not limited to, proteins that create pores in the plasma membrane for either equilibrative transport (e.g. the members of the equilibrative nucleoside transporters, ENTs) or concentrative transport (e.g. the members of the concentrative nucleoside transporters, CNTs) of materials from the extracellular to the intracellular environment. A channel can also include ion channels.

As used herein, a “cell membrane” can mean the plasma membrane surrounding a cell. It can also mean the nuclear membrane within a cell, the endoplasmic reticulum, the Golgi apparatus, the mitochondrial membrane or any other lipid-based membrane.

The term “cellular transport” or “intracellular transport” refers to the action of moving a compound across the cell membrane, thereby having the compound present inside the cell. The cellular transport, in the context of transporting HSPs, can be done by conjugating the HSPs with a compound, e.g. a (cellular) transporter or a transporting compound (or molecule) that is capable of transporting the HSPs into the cells. Some exemplary compounds of cellular transporter include an antibody 3E10 or fragment thereof and annexin.

An “annexin” is a protein characterised by its ability to bind phospholipid, particularly anionic phospholipid, in a calcium dependent manner. Annexins are also characterised by a 70 amino acid repeat sequence called an annexin repeat. The basic structure of an annexin has two major domains. The first is located at the COOH terminal and is called the “core” region. The second is located at the NH₂ terminal and is called the “head” region. The core region consists of an alpha helical disk. The convex side of this disk has type 2 calcium-binding sites important for allowing interaction with the phospholipids at the plasma membrane.

As used herein, “immunoglobulin” (also known as “antibody”) is a protein produced by plasma cells that is used by the immune system to identify and neutralize pathogens such as bacteria and viruses via a specific interaction with an antigen. The protein is typically made of basic structural units—each with two large heavy chains and two small light chains. The fragment crystallisable (or Fc) region of an immunoglobulin can have two heavy chains that contribute two or three constant domains depending on the class of the antibody. The Fc region can ensure that each antibody generates an appropriate immune response for a given antigen. The fragment variable (or Fv) region of an immunoglobulin can have one heavy chain and one light chain. The amino acids sequence in the complementarity determining regions (CDRs) of these chains give the antibody its specificity for its antigen target. Wild-type antibodies can have two Fv regions and one Fc region.

As used herein, “stress” refers to a change in the cell's normal functioning that can lead to protein misfolding, protein aggregation and/or to apoptosis (also known as programmed cell death). Stress can be the result of a shift in pH, ambient temperature, a change in available oxygen (leading to hypoxia, hyperoxia or oxidative stress), an increase in reactive oxygen species (ROS), an increase in reactive nitrogen species (RNS) or any of a number of other changes in physical parameters outside the optimal operating conditions of a cell.

As used herein, “trauma” refers to any insult to cells and tissues caused by mechanical or acoustic impact, chemical or biological toxins, and radiological or electromagnetic radiation. The trauma can be caused by a variety of methods, including but not limited to injection, ingestion, inhalation and topical exposure.

As used herein, “cancer” is meant a family of diseases that involve abnormal cell growth with the potential to invade or spread to other parts of the body. A tumor is a group of cells that are transformed and grow without normal cell regulation. Reference to treating cancer or treating a tumor can include cells of a tumor mass and cells of the tumor microenvironment, such as tumor vasculature and stromal cells (e.g. fibroblasts and immune cells) for example.

As used herein, “treatment” in the context of disease or condition is meant that at least an amelioration of the symptoms associated with the condition afflicting an individual is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g preventing disease progression to a harmful state; (ii) inhibition, that is, arresting the development or further development of clinical symptoms, e.g. mitigating or completely inhibiting an active disease, e.g. so as to decrease tumor load, which decrease can include elimination of detectable cancerous cells, or so as to protect against disease caused by bacterial infection, which protection can include elimination of detectable bacterial cells; and/or (iii) relief, that is, causing the regression of clinical symptoms.

As used herein, “effective amount” of a composition as provided herein is intended to mean a non-lethal but sufficient amount of the composition to provide the desired utility. For instance, in order to elicit a favorable response in a subject to treat a disease, the effective amount is the amount which reduces, eliminates or diminishes the symptoms associated with the disorder. As well be understood by a person having ordinary skill in the art, the exact amount required will vary from subj ect to subject, depending on the species, age, and general condition of the subject, the severity of the condition or disease that is being treated, the particular composition used, its mode of administration, and the like. Accordingly, an appropriate effective amount can be determined by one of ordinary skill in the art using routine optimization procedure.

As used herein, the phrase “pharmaceutically acceptable excipient” as used herein refers to any suitable substance which provides a pharmaceutically acceptable compound for administration of a compound(s) of interest to a subject. “Pharmaceutically acceptable excipient” encompasses substances referred to as pharmaceutically acceptable diluents, pharmaceutically acceptable additives and pharmaceutically acceptable carriers.

The terms “individual”, “subject”, or “host” as used herein refers to humans, mammals and other animals in the context of treatment using the composition of the present disclosure. In some cases, the subject being a human can be a patient.

As used herein, statistical significance based on the Student's t-test to reject the null hypothesis is defined using calculated probability values (“p value”) from the GraphPad Prism software package where >0.05 is not significant, ≤0.05 is significant and highlighted by an asterisk on graphs (*), ≤0.01 is significant and highlighted by two asterisks on graphs (**), ≤0.001 is highly significant and highlighted by three asterisks on graphs (***).

As used herein, “His-tag” refers to a polypeptide sequence of 5 or 6 histidine amino acids operably linked to a protein sequence. In some embodiments, the His-tag is placed at the ends of the protein sequence. In some embodiments, the His-tag is placed within the protein sequence. The histidine tag or “His-tag” exploits the ability of such a sequence to bind metal ions, such as nickel and cobalt, under physiological buffer conditions (pH 6-8). Proteins including a His-tag which bind a nickel- or cobalt-containing resin can be eluted with a low pH buffer (pH<5) as part of a purification process.

It is understood that aspects and embodiments of the disclosure described herein include “comprising,” “consisting,” and “consisting essentially of” aspects and embodiments.

Headings, e.g., (a), (b), (i) etc., are presented merely for ease of reading the specification and claims. The use of headings in the specification or claims does not require the steps or elements be performed in alphabetical or numerical order or the order in which they are presented.

### Modified Heat Shock Proteins

Some embodiments of the present disclosure relate to a genetically modified form of heat shock protein (HSP) such as HSP72 which can be produced in eukaryotic cells and released through the secretory pathways of these cells without being glycosylated. The methodology employed can be used to modify proteins in other HSP classes. The disclosure also describes uses for the modified HSP72 and other HSPs to treat ocular, neurological, muscular, hepatic, renal, integumentary, cardiovascular and pulmonary conditions and traumas.

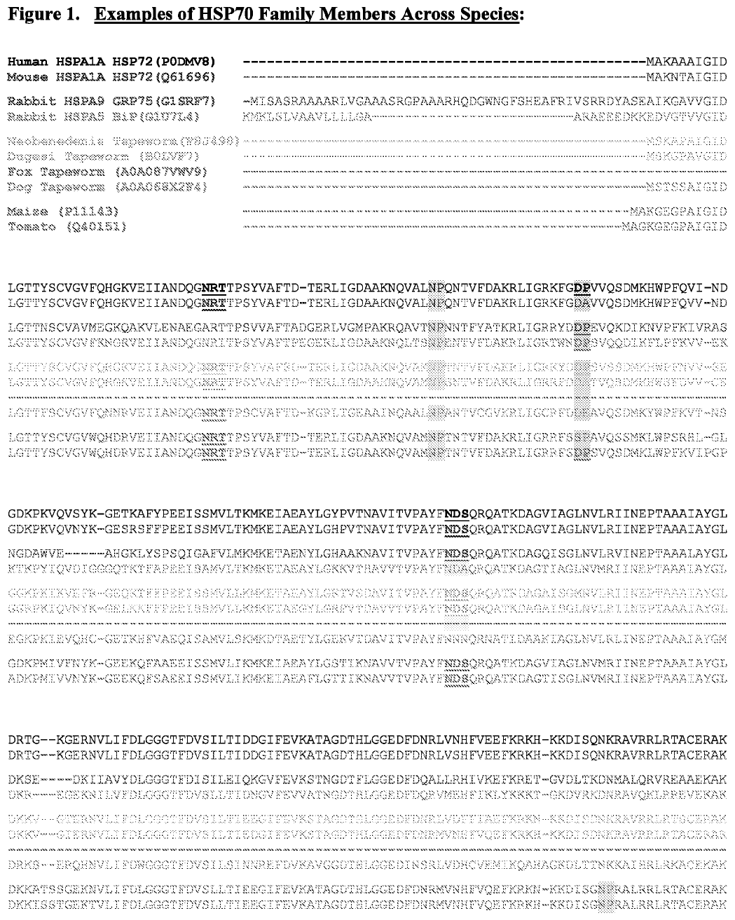

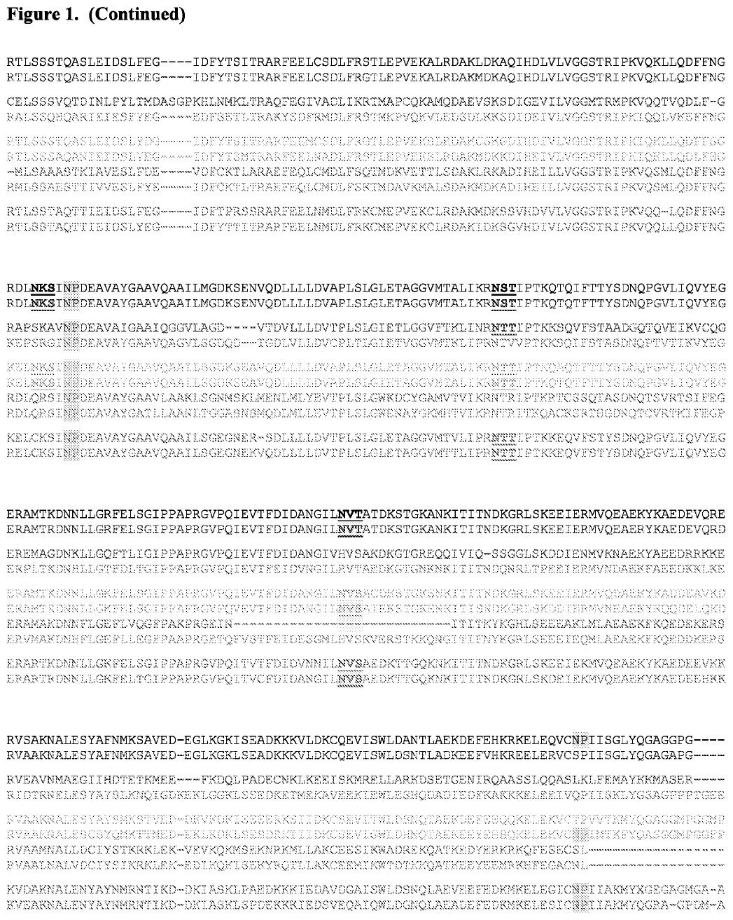

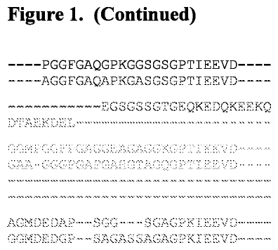

In another aspect, some embodiments of the disclosure relate to the production of HSPs for use as therapeutics. Such use has been considered complicated by the unique three-dimensional structure of these pleiotropic proteins which are designed to accommodate multiple domains with different activities. For example, members of the HSP70 family of proteins tend to have an N-terminal ATP-binding domain, and a bipartite C-terminal peptide binding domain comprised of a (3-sandwich and an α-helical structure. This multi-domain structure is conserved among HSP70 family members ranging from the DnaK homologs in Escherichia coli and Thermus thermophilus, to the Ssal homolog in Saccharomyces cerevisiae and the HSP70, HSC70 and BiP homologs in mammals [Daugaard, M., Rohde, M., and Jaattela, M. (2007) The heat shock protein 70 family: Highly homologous proteins with overlapping and distinct functions. FEBS Lett. 581 (19): 3702-3710]. FIG. 1 illustrates this conservation across species from mammals to plants.

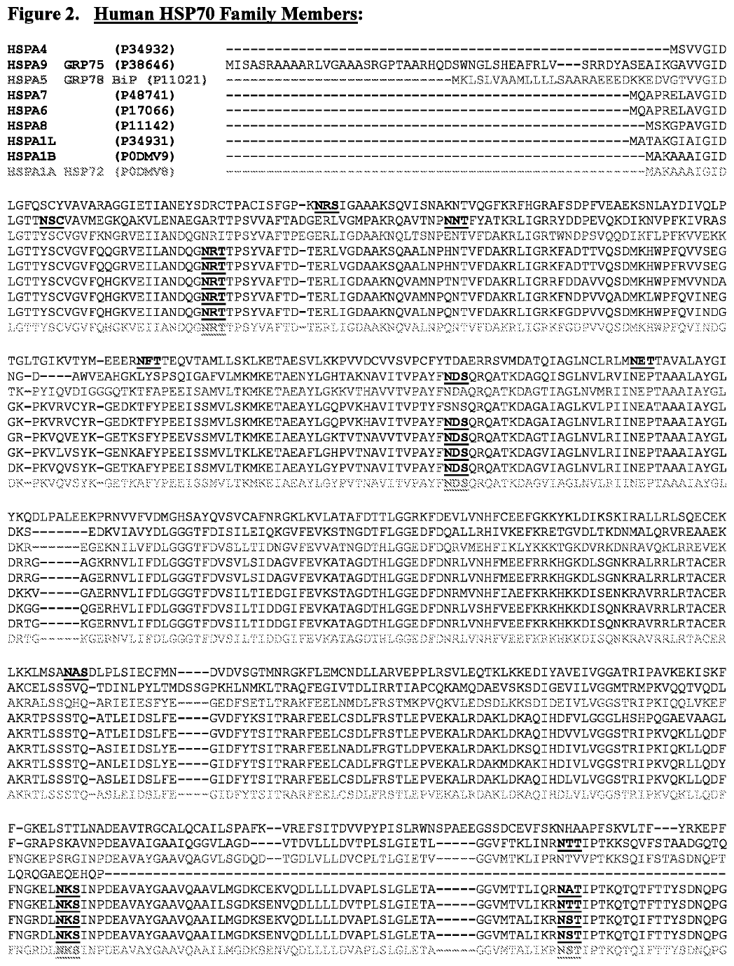

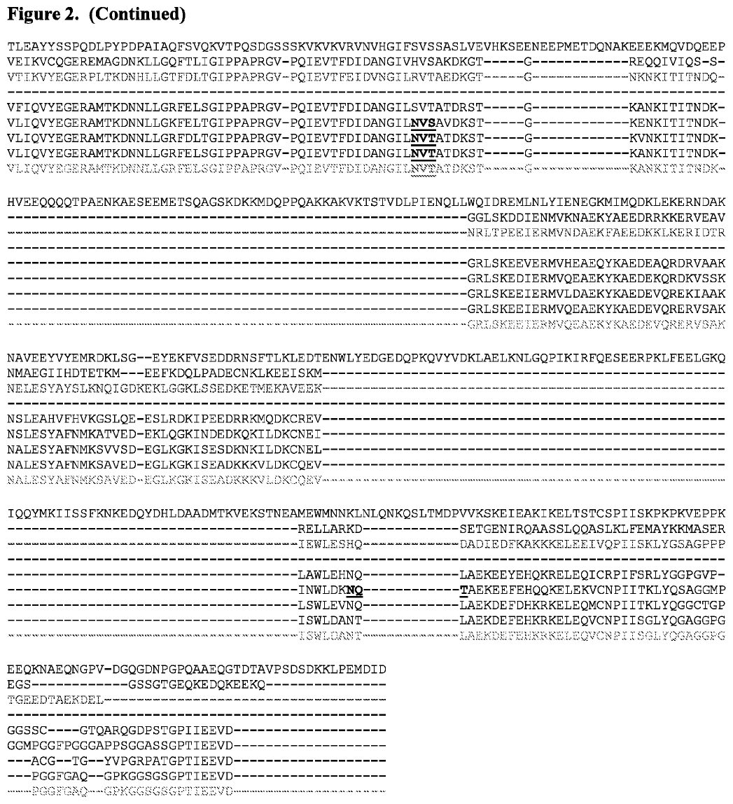

Heat shock proteins or HSPs are intracellular denizens present in the cytosol, nucleus and, in some cases such as the variant known as BiP, localized in the endoplasmic reticulum. There are multiple families of heat shock proteins, some designated according to the approximate molecular weight of the first variant to be described in that family. Hence, the families known as HSP27, HSP70, HSP90, etc., each has multiple variants in higher animals. For example, the HSP70 family in humans consists of at least 12 genes expressing proteins ranging in molecular weight from 66 kD to 78 kD [Tavaria, M., Gabriele, T., Kola, I., and Anderson, R. L. (1996) A hitchhiker's guide to the human Hsp70 family. Cell Stress. Chaperones. 1 (1): 23-28]. FIG. 2 illustrates the variation in primary sequence for certain human HSP70s. Different members of a family bear modifications that have evolved for adaptation to specific organelles and cellular environments.

Production of HSPs for research purposes has been achieved in the bacterium Escherichia coli (E. coli); however, the quantities recovered from bacterial inclusion bodies after the process of refolding the proteins is too minor to support any large scale commercial development for clinical use. Thus, some have turned to eukaryotic cells for production, investigating the use of fungal, insect and mammalian systems. For example, early production of human HSP72, a member of the HSP70 family (FIG. 2) has been achieved in the yeast Pichia pastoris both as a wild-type protein and a fusion attached to a targeting protein.

One such targeting protein is a single chain Fv fragment (scFv) derived from the 3E10 monoclonal antibody. 3E10 binds DNA allowing 3E10 penetration through a nucleoside salvage channel found in most cells, known as the equilibrative nucleoside transporter 2 (ENT2) [Hansen, J. E., Tse, C. M., Chan, G., Heinze, E. R., Nishimura, R. N., and Weisbart, R. H. (2007) Intranuclear protein transduction through a nucleoside salvage pathway. J Biol. Chem. 282 (29): 20790-20793.] This characteristic has resulted in the development of the 3E10 antibody, and its scFv derivative, as an intracellular transport for protein therapeutics that are either chemically conjugated or genetically fused to it.

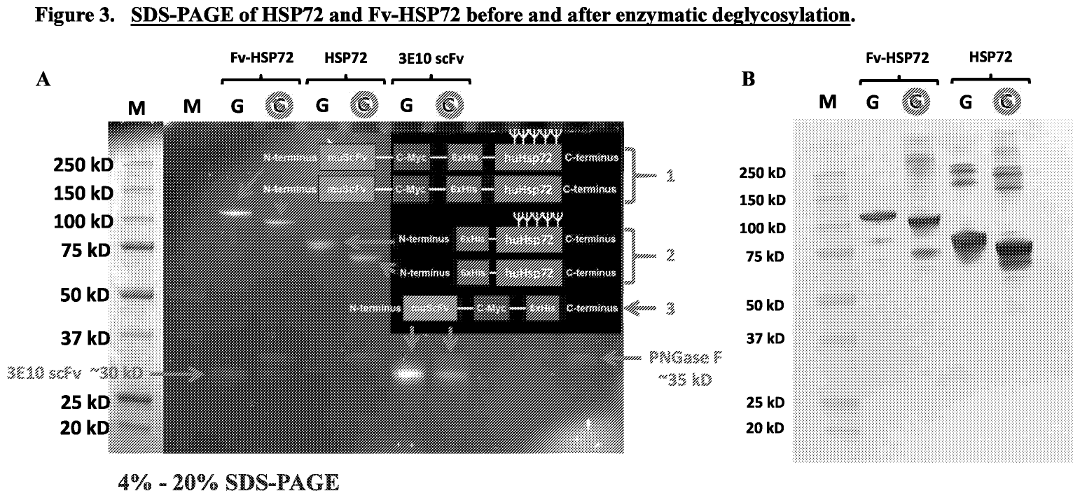

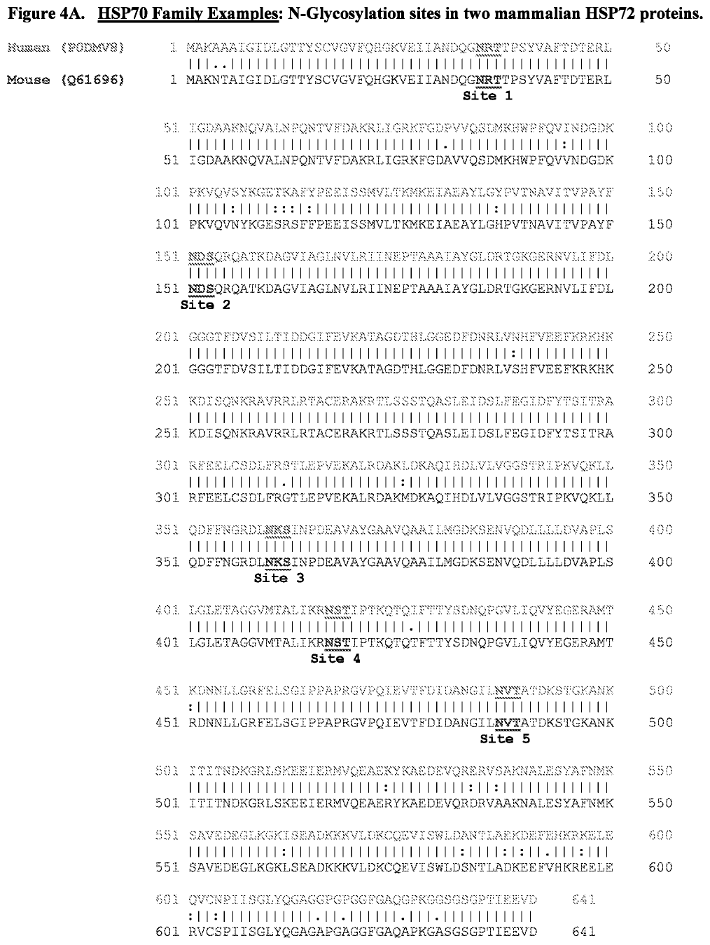

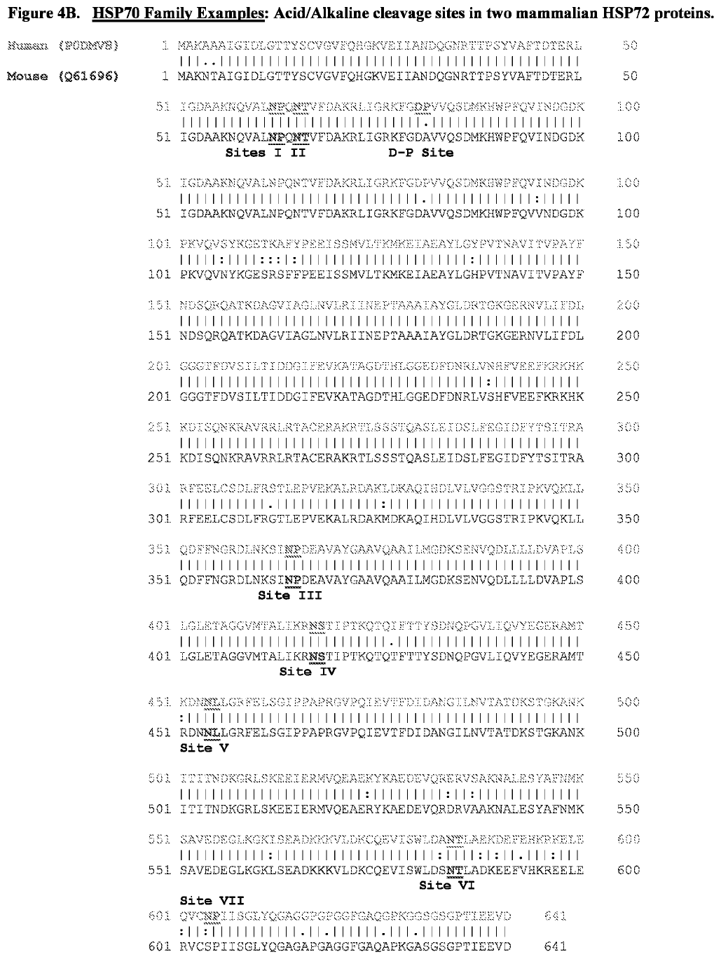

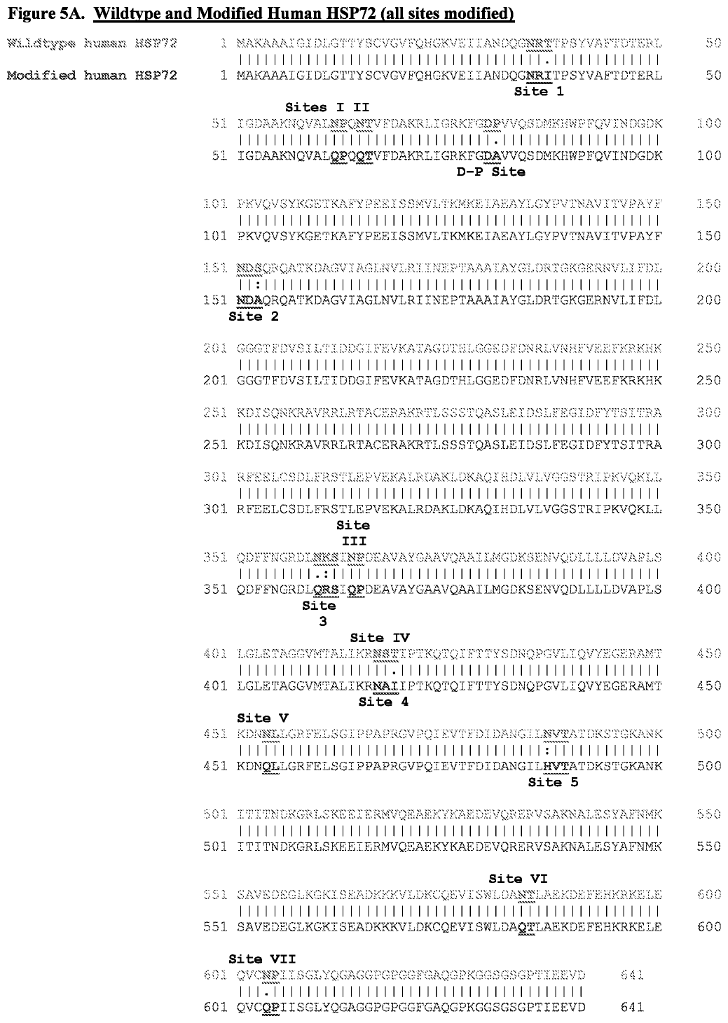

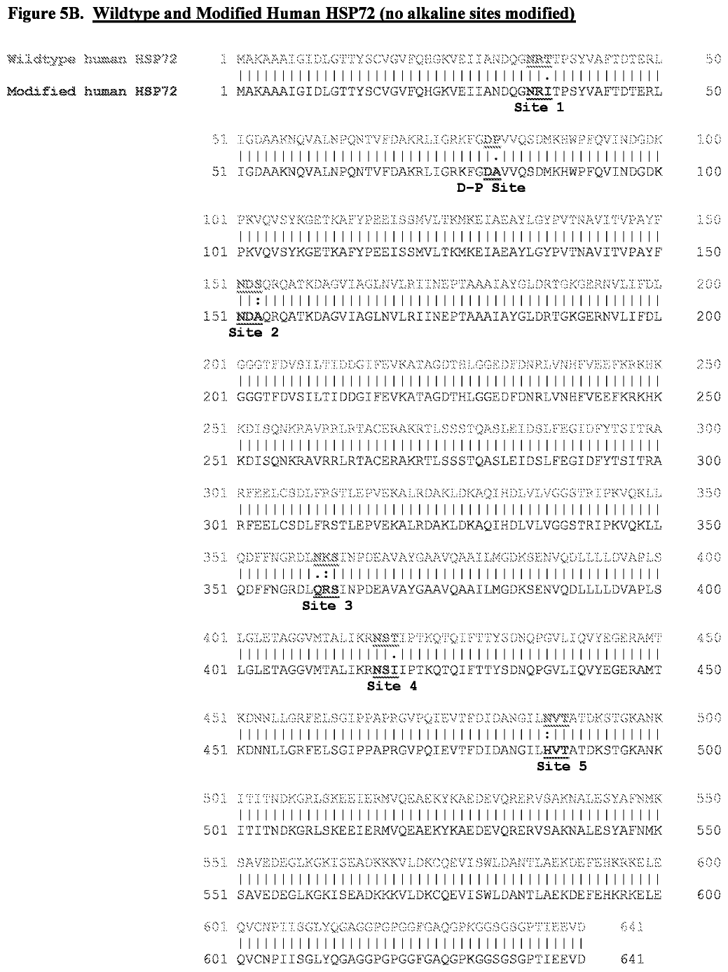

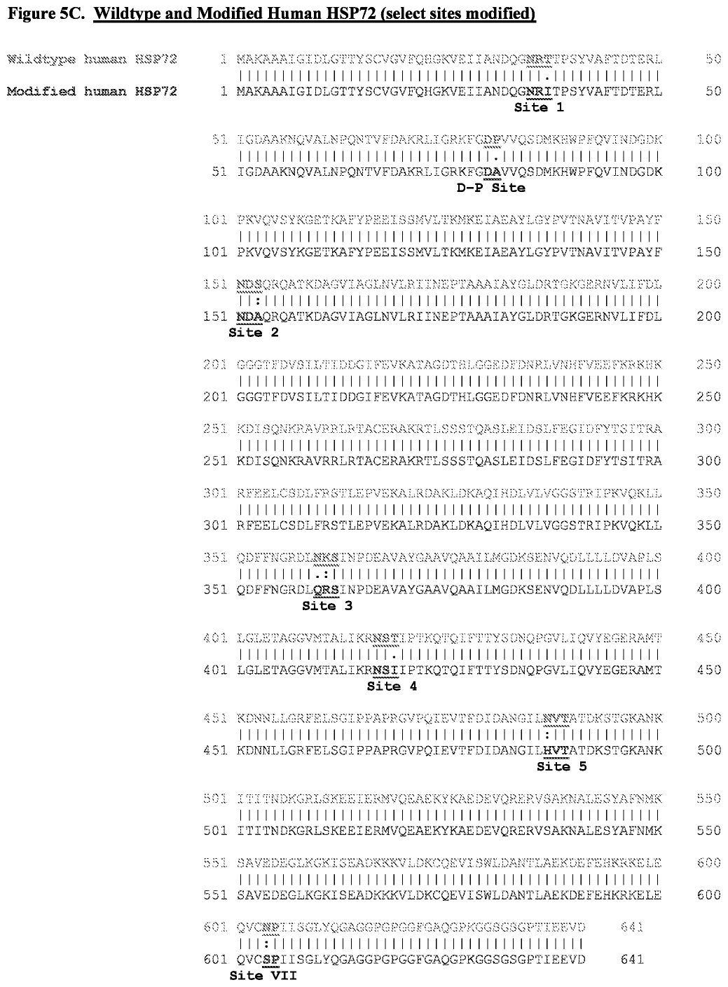

Development of the 3E10 fusion to HSP72, known in the scientific literature as “Fv-HSP70” or “Fv-HSP72”, has resulted in a cytoprotectant that can quickly be internalized into stressed cells in a wide range of traumas to rescue them from the process of apoptosis and attenuate the effects of tissue damage. However, a surprising finding during production in the eukaryotic Pichia cells was the glycosylation of the product as evidenced by shifts in mobility after treatment of wild-type HSP72 and the Fv-HSP72 fusion with an N-Glycanase (peptide-N-glycosidase F, PNGase F from Prozyme) as seen in FIG. 3. The shifts in mobility for the intact fusion molecule (Fv-HSP72) or the HSP72 alone indicate glycosylation occurs in the product secreted from Pichia. Careful evaluation of the HSP72 sequences in mammals finds five putative N-glycosylation sites (FIG. 4) as defined by the consensus sequence where an asparagine residue is followed on its carboxy end by any amino acid (except proline) and then followed by a serine or threonine subsequent to that residue (e.g. N-X-S/T) [Kornfeld, R., Kornfeld, S., (1985) Assembly of asparagine-linked oligosaccharides. Annu Rev Biochem. 54: 631-664]. On rare occasions, a cysteine in the third position (e.g. N-X-C) can also lead to asparagine glycosylation [Vance, B. A., Wu, W., Ribaudo, R. K., Segal, D. M., Kearse, K. P., (1997) Multiple dimeric forms of human CD69 result from differential addition of N-glycans to typical (Asn-X-Ser/Thr) and atypical (Asn-X-Cys) glycosylation motifs. J Biol Chem. 272(37):23117-23122; Matsui, T., Takita, E., Sato, T., Kinjo, S., Aizawa, M., Sugiura, Y., Hamabata, T., Sawada, K., Kato, K., (2011) N-glycosylation at noncanonical Asn-X-Cys sequences in plant cells. Glycobiology. 21(8):994-999]. It is the amide nitrogen on the side chain of asparagine that is glycosylated at these sites, hence the term “N-glycosylation”.

N-glycosylation occurs during protein secretion from eukaryotic cells at the N-X-S/T/C site whether those cells are fungal, insect or mammalian. Based on electrophoretic analysis of the migration patterns in FIG. 3, the Fv-HSP72 runs at ˜110 kD despite a calculated molecular weight of 100 kD. Oligosaccharide modification to proteins in fungi is characterized by the addition of oligomannose moieties at the N-X-S/T/C sites during secretion. Based on the ˜10 kD molecular weight discrepancy, it is believed all five putative sites on the HSP72 are post-translationally modified with a nine mannose sugar structure (“oligomannose-9”). The oligomannose-9 has a molecular weight of 1884 Daltons. Five such structures attached to a single HSP72 or Fv-HSP72 would increase the molecular weight by 9420 Daltons.

The functionality of a heavily mannose glycosylated HSP72 was surprising. This first generation fusion molecule, designated as RBB001 at Rubicon Biotechnology, had been tested in stroke and myocardial infarction studies and shown efficacy as a cytoprotectant, minimizing neural cell damage in a rat stroke model by 68% and cardiomyocyte damage in a rabbit infarction model by 43%. Clinical development of an Fv-HSP72 fusion with such a carbohydrate modification can be problematic. First, the carbohydrate modifications must be well-characterized and the exact structure of each carbohydrate structure at each N-X-S/T/C site determined during each production run of the molecule. As those knowledgeable in the art of glycoprotein production are aware, there is variation in the carbohydrate modifications that occur on any protein based on minor changes in the cellular environment, including changes in temperature, pH, oxidative stress, etc. Control of carbohydrate modifications on a protein is challenging and can result in significant increases in the cost of production and reductions in product yield in order to achieve very specific carbohydrate structures on each and every molecule. Furthermore, glycosylation of a protein differs in different cellular systems. The glycosylation pattern seen in fungal cells is different from those seen in insect or mammalian cells. Hence, if changes in glycosylation can affect product efficacy, the ability to transfer the product's genes from a fungal system to other cellular production systems may be limited.

In some aspects, the disclosure disclosed herein relates to creation of HSP, e.g. an HSP72 that is not glycosylated during secretion and portable from one cellular production system to another. Some embodiments provide a new modified HSP72 and fusion protein in which the modified HSP72 is fused to a 3E10 scFv fragment. The modified HSP72 in accordance with several embodiments of the present disclosure is novel and, surprisingly, functional given the complex, multi-domain 3-dimensional structure of heat shock proteins.

### Compositions of the Disclosure

Some embodiments of the disclosure relate to a composition that contains a modified heat shock protein or HSP. In some embodiments, the modified HSP has modification(s) in one or more glycosylation sites and/or one or more acid-labile cleavage sites and/or one or more alkaline-labile cleavage sites from the primary sequence of HSP, e.g. a wild-type HSP sequence, functional derivative or functional fragment thereof In some embodiments, the modifications lead to substantial reduction or block of glycosylation and/or acid-labile cleavage and/or alkaline-labile cleavage of the modified sites. In some embodiments, the composition contain a conjugate or fusion protein in which a modified HSP is associated with another compound.

In some embodiments, the glycosylation of interest generally refer to N-glycosylation. In some embodiments, the glycosylation of interest includes other types of glycosylation such as O-linked glycosylation, phosphor-serine glycosylation, C-mannosylation and glypiation. Therefore, in some embodiments, modification of a protein can include reduction or block of N-glycosylation. In some embodiments, modification of a protein can result in reduction or block of other types of glycosylation as well along with reduction or block of N-glycosylation.

In some embodiments, the “N-glycosylation” includes a site having the amino acid sequence of N-X-S/T/C, wherein X is any amino acid sequence except proline. In some embodiments, X is an amino acid selected from the group consisting of R, D, K, S, T, V, and N. In some embodiments, modifications such as, for example, deletions and/or substitutions of any one or more of amino acid from the three positions, i.e. N, X and S/T/C can occur in a way that N-glycosylation of the modified protein is substantially reduced or completely blocked. In some embodiments, such modification include non-conservative modification of one or more of the amino acids.

In some embodiments, at least about 10%, at least about 20%, at least about 30%, at least about 40%, at least about 50%, at least about 60%, at least about 70%, at least about 80%, at least about 90%, at least about 95%, at least about 99%, or at least about 100% of the total number of the N-glycosylation sites in the modified heat shock protein is modified.

In some embodiments, about 10% to about 90%, about 20% to about 80%, about 30% to about 70%, about 40% to about 60%, or about 20% to about 50%, about 30% to about 60%, about 40% to about 70%, about 50% to about 80%, about 60% to about 90%, about 70% to about 100% of the total number of the N-glycosylation sites in the modified heat shock protein is modified.

In some embodiments, about 10%, about 20%, about 30%, about 40%, about 50%, about 60%, about 70%, about 80%, about 90%, about 95%, about 99%, or about 100% of the total number of the N-glycosylation sites is modified in said heat shock protein or fragment thereof

In some embodiments, while the modification on one or more of the N-glycosylation sites, e.g., N-X-S/T/C sites, in HSP may affect the level of N-glycosylation, it may not substantially affect other properties of the HSP. In one non-limiting exemplary embodiment, a modified HSP in which one or more N-glycosylation sites are modified yet still retains at least about 10%, at least about 20%, at least about 30%, at least about 40%, at least about 50%, at least about 60%, at least about 70%, at least about 80%, at least about 90%, at least about 95%, at least about 98%, at least about 99%, or about 100% of the activity that the corresponding unmodified HSP protein exhibits. In some embodiments, the modified HSP retains about 10% to about 90%, about 20% to about 80%, about 30% to about 70%, about 40% to about 60%, or about 20% to about 50%, about 30% to about 60%, about 40% to about 70%, about 50% to about 80%, about 60% to about 90%, about 70% to about 100% of the activity of the corresponding unmodified HSP protein. In some embodiments, the modified HSP retains about 10%, about 20%, about 30%, about 40%, about 50%, about 60%, about 70%, about 80%, about 90%, about 95%, about 99%, or about 100% of the activity of the corresponding unmodified HSP protein. In some embodiments, the modified HSP retains about 10%, about 20%, about 30%, about 40%, about 50%, about 60%, about 70%, about 80%, about 90%, about 95%, about 98%, about 99%, or about 100% of the activity of the corresponding unmodified HSP protein.

In a non-limiting example, where the activity of interest is a capability of HSP to inhibit cell death, the modified HSP may still contain at least about 10% to about 100% of the activity of the corresponding unmodified HSP in reducing cell death. In some embodiments, the modified HSP in which one or more N-glycosylation sites are modified has at least about 10%, at least about 20%, at least about 30%, at least about 40%, at least about 50%, at least about 60%, at least about 70%, at least about 80%, at least about 90%, at least about 95%, at least about 98%, at least about 99%, or at least about 100% of the activity that the corresponding unmodified HSP protein has in inhibiting cell death. In some embodiments, the modified HSP has about 10% to about 90%, about 20% to about 80%, about 30% to about 70%, about 40% to about 60%, or about 20% to about 50%, about 30% to about 60%, about 40% to about 70%, about 50% to about 80%, about 60% to about 90%, about 70% to about 100% of the activity that the corresponding unmodified HSP protein has in inhibiting cell death. In some embodiments, the modified HSP has about 10%, about 20%, about 30%, about 40%, about 50%, about 60%, about 70%, about 80%, about 90%, about 95%, about 99%, or about 100% of the activity that the corresponding unmodified HSP protein has in inhibiting cell death. In some embodiments, the modified HSP has about 10%, about 20%, about 30%, about 40%, about 50%, about 60%, about 70%, about 80%, about 90%, about 95%, about 98%, about 99%, or about 100% of the activity that the corresponding unmodified HSP protein has in inhibiting cell death.

The N-glycosylation site can generally be any N-glycosylation site known in the art. In some embodiments, the N-glycosylation site comprises the following amino acid sequence: N-X-S/T/C, wherein X is any amino acid except proline. Accordingly, in some embodiments, the N-glycosylation site comprises the amino acid sequence N-X-S, wherein X is any amino acid except proline. In some embodiments, the N-glycosylation site comprises the amino acid sequence N-X-T, wherein X is any amino acid except proline. In some embodiments, the N-glycosylation site comprises the amino acid sequence N-X-C, wherein X is any amino acid except proline. In some embodiments, the N-glycosylation sites in the HSP polypeptide are the same. In some embodiments, the N-glycosylation sites in the HSP polypeptide are different. In some embodiments, among the amino acids of the N-glycosylation site of N-X-S/T/C, the first positioned amino acid N, i.e. asparagine (Asn), which is a neutral amino acid with a polar side chain, is substituted by another amino acid such that N-glycosylation in the modified site is reduced. In some embodiments, N-glycosylation in the modified site is blocked. In some embodiments, asparagine in the N-X-S/T/C site is substituted by glutamine (Gln or Q) which is also a neutral amino acid with a polar side chain. In some embodiments, asparagine in the N-X-S/T/C site is substituted by histidine (His or H) which is also a neutral amino acid with a polar side chain at a pH>6 and a charged side chain at a pH<6. In some embodiments, asparagine in the N-X-S/T/C site is substituted bycysteine (Cys or C), a neutral amino acid with a polar side chain when in a reduced state (e.g., when not in a state of oxidation). In some embodiments, any one of the amino acids in the N-glycosylation site can be substituted by a natural or non-natural amino acid can replace. In some embodiments, a conservative amino acid replacement from one amino acid with a neutral side chain to another amino acid with a neutral side chain is provided. For example, replacement with those amino acids listed in the Aliphatic, Aromatic, Cyclic, Hydroxyl or Sulfur/Selenium-containing Groups in TABLE 1 above.

In some embodiments, among the amino acids of the N-glycosylation site of N-X-S/T/C, the second positioned residue X, which is any amino acid except proline, can be substituted in a way that results in reduction or abolition of N-glycosylation in the modified site without interfering with overall protein activity. In some embodiments, the second positioned residue X is replaced with a proline which, when substituted into that second position, disrupts N-glycosylation at the N-X-S/T/C site. Without being bound to any particular theory, it is contemplated that one can possibly create a non-glycosylated HSP by only replacing the second positioned amino acid with prolines at every N-X-S/T/C site throughout the HSP molecule, if it does not disrupt the structure of the HSP sufficiently to reduce or eliminate activity. However, introduction of these new N-P sites may result in a modified HSP molecule that is sensitive to alkaline pH conditions and may not be stable.

In some embodiments, among the amino acids of the N-glycosylation site of N-X-S/T/C, the third positioned amino acid S, T or C, i.e. serine (Ser), Threonine (Thr) or Cysteine (Cys), respectively can be substituted by another amino acid such that N-glycosylation in the modified site is reduced or blocked. In some embodiments, threonine, which is a neutral amino acid with a polar chain in the N-X-S/T/C site is substituted byisoleucine (Ile or I) which has a non-polar side chain. In some embodiments, threonine is substituted by valine (Val or V). In some embodiments, serine, which is a neutral amino acid with a polar chain in the N-X-S/T/C site is substituted by to alanine (Ala or A) which has a non-polar side chain. In some embodiments, any natural or non-natural amino acid could replace the third positioned residue S/T/C. In some embodiments, a replacement of one or more members of the Hydroxyl or Sulfur/Selenium-containing Group as indicated in TABLE 1 above with members of other Groups (Aliphatic, Aromatic, Cyclic, Basic or Acidic and their Amides) is provided.

In some embodiments, one or more of the amino acid can be deleted from an N-glycosylation site, e.g., N-X-S/T/C site, to reduce or block N-glycosylation on the site. In some embodiments, one amino acid is deleted from the N-glycosylation site. In some embodiments, two amino acids are deleted from the N-glycosylation site. In some embodiments, all three amino acids are deleted from the N-glycosylation site. In some embodiments, one or more amino acid can be added before or after the N-X-S/T/C site or within the site such that N-glycosylation on the site is reduced or blocked. In some embodiments, one amino acid is added to the N-glycosylation site. In some embodiments, two amino acids are added to the N-glycosylation site. In some embodiments, three, four, five, or six amino acids are added to the N-glycosylation site. In some embodiments, more than one modifications, e.g. substitution, deletion and addition of one or more amino acids, can be made to the N-X-S/T/C site and/or in the proximity thereof (e.g. one, two, three, or four amino acids before or after the site) to reduce or block N-glycosylation on the site.

In some embodiments where the primary HSP protein sequence has one or more N-glycosylation sites, the modification of the HSP protein include modification of one N-glycosylation site or modification of more than one glycosylation sites. In some embodiments, all of the N-glycosylation sites in the primary sequence are modified such that the modified protein is not glycosylated at all.

In some embodiments, a site for acid-labile cleavage within an HSP primary sequence comprises an amino acid sequence that can be cleaved when the sequence is subject to an acidic environment. In some embodiments, the acid-labile cleavage site can be cleaved or cut when it is exposed to a low pH, e.g. pH of lower than or equal to about 7. In some embodiments, the low pH or acid pH causing the cleavage on the acid-labile cleavage site is about 7 or less, about 6.5 or less, about 6 or less, about 5.5 or less, about 5 or less, about 4.5 or less, about 4 or less, about 3.5 or less, about 3 or less, about 2.5 or less or about 2 or less. In some embodiments, the acid-labile cleavage site can be cleaved when the surrounding pH is about 6 or lower.

In some embodiments, a site for alkaline-labile cleavage within an HSP primary sequence comprises an amino acid sequence that can be cleaved when the sequence is subject to an alkaline environment. In some embodiments, the alkaline-labile cleavage site is cleaved or cut when it is exposed to a high pH, e.g. pH of higher than or equal to about 7. In some embodiments, the high pH or alkaline pH causing the cleavage on the alkaline-labile cleavage site is about 7 or more, about 7.5 or more, about 8 or more, about 8.5 or more, about 9 or more, about 9.5 or more, about 10 or more, about 10.5 or more, about 11 or more, about 11.5 or more, about 12 or more, about 12.5 or more, about 13 or more, about 13.5 or more, or about 14. In some embodiments, the alkaline-labile cleavage site can be cleaved when the surrounding pH is about 8 or higher.

Generally, the acid-labile cleavage site can be any one of the acid-labile cleavage sites known in the art. In some embodiments, the acid-labile cleavage sites in the HSP polypeptide are the same. In some embodiments, the acid-labile cleavage sites in the HSP polypeptide are different. In some embodiments, the “acid-labile cleavage site” includes the amino acid sequence of D-P. In some embodiments, deletion and/or substitution of any one or more of amino acid in these two positions, i.e. D and P, can occur to substantially reduce or completely block the acid-labile cleavage at the site. In some embodiments, such modifications include non-conservative modification of one or more residues.

Generally, the alkaline-labile cleavage site can be any one of the alkaline-labile cleavage sites known in the art. In some embodiments, the alkaline-labile cleavage sites in the HSP polypeptide are the same. In some embodiments, the alkaline-labile cleavage sites in the HSP polypeptide are different. In some embodiments, the “alkaline-labile cleavage site” includes the amino acid sequence selected from the group consisting of N-P, N-L, N-S, and N-T. In some embodiments, deletion and/or substitution of any one or more of amino acid in these two positions, e.g. N and P, can occur to substantially reduce or completely block the alkaline-labile cleavage at the site. In some embodiments, such modification include non-conservative modification of one or more residues.

In some embodiments, at least about 10%, at least about 20%, at least about 30%, at least about 40%, at least about 50%, at least about 60%, at least about 70%, at least about 80%, at least about 90%, at least about 95%, at least about 99%, or at least about 100% of the total number of the acid-labile sites in the modified heat shock protein is modified. In some embodiments, about 10% to about 90%, about 20% to about 80%, about 30% to about 70%, about 40% to about 60%, or about 20% to about 50%, about 30% to about 60%, about 40% to about 70%, about 50% to about 80%, about 60% to about 90%, about 70% to about 100% of the total number of the acid-labile sites in the modified heat shock protein is modified. In some embodiments, about 10%, about 20%, about 30%, about 40%, about 50%, about 60%, about 70%, about 80%, about 90%, about 95%, about 99%, or about 100% of the total number of the acid-labile sites in the modified heat shock protein is modified. In some embodiments, about 10%, about 20%, about 30%, about 40%, about 50%, about 60%, about 70%, about 80%, about 90%, about 95%, about 98%, about 99%, or about 100% of the total number of the acid-labile sites in the modified heat shock protein is modified.

In some embodiments, at least about 10%, at least about 20%, at least about 30%, at least about 40%, at least about 50%, at least about 60%, at least about 70%, at least about 80%, at least about 90%, at least about 95%, at least about 99%, or at least about 100% of the total number of the alkaline-labile sites in the modified heat shock protein is modified. In some embodiments, about 10% to about 90%, about 20% to about 80%, about 30% to about 70%, about 40% to about 60%, or about 20% to about 50%, about 30% to about 60%, about 40% to about 70%, about 50% to about 80%, about 60% to about 90%, about 70% to about 100% of the total number of the alkaline-labile sites in the modified heat shock protein is modified. In some embodiments, about 10%, about 20%, about 30%, about 40%, about 50%, about 60%, about 70%, about 80%, about 90%, about 95%, about 99%, or about 100% of the total number of the alkaline-labile sites in the modified heat shock protein is modified. In some embodiments, about 10%, about 20%, about 30%, about 40%, about 50%, about 60%, about 70%, about 80%, about 90%, about 95%, about 98%, about 99%, or about 100% of the total number of the alkaline-labile sites in the modified heat shock protein is modified.

In some embodiments, while the modification of one or more of D-P sites in HSP may affect the level of acid-labile cleavage, it does not substantially affect other properties of the HSP. In some embodiments, a modified HSP in which one or more acid-labile cleavage sites are modified still retains at least about 10%, about 20%, about 30%, about 40%, about 50%, about 60%, about 70%, about 80%, about 90%, about 95%, about 98%, about 99%, or about 100% of the activity that the corresponding unmodified HSP protein exhibits. In some embodiments, while the modification of one or more of N-P, N-L, N-S or N-T sites in HSP may affect the level of alkaline-labile cleavage, it does not substantially affect other properties of the HSP. In some embodiments, a modified HSP in which one or more alkaline-labile cleavage sites are modified still retains at least about 10%, about 20%, about 30%, about 40%, about 50%, about 60%, about 70%, about 80%, about 90%, about 95%, about 98%, about 99%, or about 100% of the activity that the corresponding unmodified HSP protein exhibits.

In a non-limiting example, where the activity of interest is a capability of HSP to inhibit cell death, the modified HSP still has at least about 10% to about 100% of the activity of the corresponding unmodified HSP in reducing cell death. In some embodiments, the modified HSP in which one or more acid-labile cleavage sites and/or alkaline-labile cleavage sites are modified has at least about 10%, about 20%, about 30%, about 40%, about 50%, about 60%, about 70%, about 80%, about 90%, about 95%, about 98%, about 99%, or about 100% of the activity that the corresponding unmodified HSP protein has in inhibiting cell death. In some embodiments, the modified HSP has about 10% to about 90%, about 20% to about 80%, about 30% to about 70%, about 40% to about 60%, or about 20% to about 50%, about 30% to about 60%, about 40% to about 70%, about 50% to about 80%, about 60% to about 90%, about 70% to about 100% of the activity that the corresponding unmodified HSP protein has in inhibiting cell death. In some embodiments, the modified HSP has about 10%, about 20%, about 30%, about 40%, about 50%, about 60%, about 70%, about 80%, about 90%, about 95%, about 99%, or about 100% of the activity that the corresponding unmodified HSP protein has in inhibiting cell death. In some embodiments, the modified HSP has about 10%, about 20%, about 30%, about 40%, about 50%, about 60%, about 70%, about 80%, about 90%, about 95%, about 98%, about 99%, or about 100% of the activity that the corresponding unmodified HSP protein has in inhibiting cell death.

In some embodiments, among the amino acids from the acid-labile cleavage site of D-P, the first positioned amino acid D, i.e. aspartic acid (Asp), is substituted by another amino acid such that acid-labile cleavage in the modified site is reduced. In some embodiments, the acid-labile cleavage in the modified site is blocked. Generally, aspartic acid in the D-P site can be substituted by any natural or non-natural amino acid. In some embodiments, a replacement with Serine (Ser or S) or any member of the Aliphatic Group is provided. In some embodiments, replacement with asparagine (Asn or N) is not preferred.

In some embodiments, among the amino acids from the acid-labile cleavage site of D-P, the second positioned amino acid P, i.e. proline (Pro), is substituted by another amino acid such that acid-labile cleavage in the modified site can be reduced. In some embodiments, the acid-labile cleavage in the modified site is blocked. Generally, proline in the D-P site can be substituted by any natural or non-natural amino acid. In some embodiments, a replacement with alanine (Ala or A) or any other Aliphatic Group member is provided. In some embodiments, the proline substituted by a glutamic acid (Glu or E).

In some embodiments, among the amino acids from the alkaline-labile cleavage sites of N-P, N-L, N-S or N-T the first positioned amino acid N, i.e. asparagine (Asn), is substituted by another amino acid such that alkaline-labile cleavage in the modified site is reduced. In some embodiments, the alkaline-labile cleavage in the modified site is blocked. Generally, asparagine can be substituted by any natural or non-natural amino acid. In some embodiments, a replacement with glutamine (Gln or Q), serine (Ser or S), threonine (Thr or T) or any member of the Aliphatic Group is provided. In some embodiments, replacement with aspartic acid (Asp or D) is not preferred.

In some embodiments, among the amino acids from the alkaline-labile cleavage sites of N-P, N-L, N-S or N-T the second positioned amino acid P, L, S or T, i.e. proline (Pro), leucine (Leu), serine (Ser) or threonine (Thr), respectively, is substituted by another amino acid such that alkaline-labile cleavage in the modified site can be reduced. In some embodiments, the alkaline-labile cleavage in the modified site is blocked. Generally, proline can be substituted by any natural or non-natural amino acid. In some embodiments, a replacement with alanine (Ala or A) or any other Aliphatic Group member, except leucine, is provided. In some embodiments, the proline is substituted by a lysine (Lys or K).

In some embodiments, one or more of amino acid are deleted from the D-P site to reduce or block acid-labile cleavage on the site. In some embodiments, one or more amino acid are added before or after the D-P site or within the site such that acid-labile cleavage on the site is reduced or blocked. In some embodiments, more than one modifications, e.g. substitution, deletion and addition of one or more amino acids, are made to the D-P site or proximity thereof (e.g. before or after the site) to reduce or block acid-labile cleavage on the site.

In some embodiments, one or more of amino acid are deleted from the N-P, N-L, N-S or N-T site(s) to reduce or block alkaline-labile cleavage on the site(s). In some embodiments, one or more amino acid are added before and/or after the N-P, N-L, N-S or N-T site(s) or within the site(s) such that alkaline-labile cleavage on the site(s) is reduced or blocked. In some embodiments, more than one modifications, e.g. substitution, deletion and addition of one or more amino acids, are made to the alkaline-labile site(s) and/or in the proximity thereof (e.g. one, two, three, or four residues before or after the site) to reduce or block alkaline-labile cleavage on the site(s).

In some embodiments where the primary protein sequence has one or more acid-labile cleavage sites, the modified protein include modification of one acid-labile cleavage site. In some embodiments, the modified protein include modifications of more than one sites. In some embodiments, all of the acid-labile cleavage sites in the primary sequence are modified such that the modified sites are not cleaved under a low pH. In some embodiments, the low pH causing the cleavage on the acid-labile cleavage site is about 7 or less, about 6.5 or less, about 6 or less, about 5.5 or less, about 5 or less, about 4.5 or less, about 4 or less, about 3.5 or less, about 3 or less, about 2.5 or less or about 2 or less. In some embodiments, the acid-labile cleavage site is cleaved when the surrounding pH is about 6 or lower.

In some embodiments where the primary protein sequence has one or more alkaline-labile cleavage sites, the modified protein include modification of one alkaline-labile cleavage site. In some embodiments, the modified protein include modifications of more than one sites. In some embodiments, all of the alkaline-labile cleavage sites in the primary sequence are modified such that the modified sites are not cleaved under a high pH. In some embodiments, the high pH causing the cleavage on the alkaline-labile cleavage site is about 7 or more, about 7.5 or more, about 8 or more, about 8.5 or more, about 9 or more, about 9.5 or more, about 10 or more, about 10.5 or more, about 11 or more, about 11.5 or more, about 12 or more, about 12.5 or more, about 13 or more, about 13.5 or more, or about 14. In some embodiments, the alkaline-labile cleavage site is cleaved when the surrounding pH is about 8 or higher.

In some embodiments, the modification of HSP can occur on one or more amino acids that are not within or in the proximity of the glycosylation sites, acid-labile cleavage sites and alkaline-labile cleavage sites. When such modification occurs, it can be conservative modification in which substitution of an amino acid to another amino acid may result in similar properties to the original amino acid. Therefore, in some embodiments, the modified HSP has at least amino acid identity of at least about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, or any intervening number of percentage to the corresponding unmodified HSP amino acid sequence.

Conservation of HSP70 sequences across multiple kingdoms of life attests to the sensitivity of the overall 3-dimensional structure to evolutionary change (FIG. 1). By way of a non-limiting example illustrating how the modification on HSPs can be made according to some embodiments of the disclosure, the modification of HSP72 is described below. This exemplary demonstration with HSP72 is merely to illustrate certain aspects of the disclosure and therefore it should not be considered limiting a scope of the disclosure in any manner. As also described elsewhere in this application, the modification of HSP can be practiced with any of HSPs including any member of the chaperonins, HSP27, HSP40, HSP60, HSP70, HSP90, HSP105/110, small heat shock proteins and other newly emerging family of heat shock proteins identified by sequence comparison to homologous, orthologous or paralogous family members, in view of the guidance and disclosure provided herein.

In some embodiments, human HSP72, which contains five N-glycosylation sites, one acid-labile cleavage site, and up to seven alkaline-labile cleavage sites, can be modified. In some embodiments, modifications can be made to all 12 sites (N-glycosylation Site 4 and alkaline-labile Site IV overlap) on the wild-type human HSP72: 5 putative N-glycosylation sites, 7 sites susceptible to cleavage under a moderately high pH, and 1 site susceptible to cleavage under a moderately low pH (FIG. 5A). In some embodiments, any number of 0 to 5 of glycosylation sites can be modified, with any number of 0 to 7 of alkaline-labile cleavage sites, and with or without the modification of the single acid-labile cleavage site in HSP72 (e.g. FIGS. 5B and 5C).

Change to Site 1: In some embodiments, threonine-37 (T37) in HSP72 is replaced with isoleucine (137). This change replaces threonine, a neutral amino acid with a polar side chain, with a residue that has a non-polar side chain and one more carbon atom. Isoleucine can be considered only weakly similar in properties to threonine based on scoring in the PAM 250 matrix. Evolution has resulted in a similar change at this site in the human BiP protein (see FIG. 2).

Change to Site 2: In some embodiments, serine-153 (S153) in HSP72 is replaced with alanine (A153). This change replaces serine, a neutral amino acid with a polar side chain, with a residue that has a non-polar side chain. Alanine can be considered strongly similar in properties to serine based on scoring in the PAM 250 matrix. Evolution has resulted in a similar change at this site in the human BiP protein (see FIG. 2).

Change to Site 3: In some embodiments, asparagine-360 (N360) in HSP72 is replaced with glutamine (Q360). This change replaces asparagine, a neutral amino acid with a polar side chain, with another neutral amino acid, also with a polar side chain. However, due to the extra carbon atom in the side chain, the conversion from N to Q can be considered weakly similar according to the PAM 250 matrix. In some embodiments, lysine-361 (K361) in HSP72 is replaced with arginine (R361). The conversion from lysine to arginine replaces one polar side chain with another and can be considered strongly similar according to the PAM 250 matrix even though the arginine side chain has an extra amine. This site is highly conserved in numerous entries in the Uniprot and NCBI protein databases. In some embodiments, it can go down the evolutionary tree to flatworms and tapeworms before one finds some species where the NKS sequence is replaced with a QRS sequence (e.g., when compare the sequences for Neobenedenia and Dugesi at this site to those of the fox and dog tapeworms, as shown in FIG. 1). It is also contemplated, in some embodiments, to replace the asparagine with a cysteine at this site, as seen in corn and tomato plants. In some embodiments, cysteine is converted given the complications of having an unpaired, reduced sulfhydryl side chain in a protein that would need to be maintained as a monomer.

Change to Site 4: In some embodiments, threonine-419 (T419) in HSP72 is replaced with isoleucine (1419). This change replaces threonine, a neutral amino acid with a polar side chain, with a residue that has a non-polar side chain and one more carbon atom. Isoleucine can be considered only weakly similar in properties to threonine based on scoring in the PAM 250 matrix. It is contemplated, in some embodiments, to replace the threonine with valine, which also may result in a weakly similar replacement according to the PAM 250 matrix. In some embodiments, when codon-optimized for mammalian cell production, the serine-418 is encoded by the codon TCC. All valine codons contain a deoxyguanosine in the first position (the codon for valine is GTX, where X can be A, C, T or G). The juxtaposition of the C from TCC and the G from GTX can increase the chance that the CG pair could be methylated and the expression of the final gene product attenuated. Methylation of CG pairs (referred to in the literature as “CpG” methylation) can occur in regions enriched with deoxycytidine and deoxyguanosine. CpG islands are usually defined as genomic regions with increased susceptibility to methylation and are characterized by a greater than 50% G+C content in a region of at least 200 base pairs. The chances for methylation of the cytosine in the CG pair after introducing valine to replace the threonine can be more than 50:50 since an analysis showed a greater than 62% GC content in the 120 nucleotides upstream and 120 nucleotides downstream from the codon for Threonine-419. Therefore, in some embodiments, replacement of the threonine-419 is done with an isoleucine instead of valine. The mammalian optimal codon used for isoleucine-419 was ATC.

Change to Site 5: In some embodiments, asparagine-487 (N487) in HSP72 is replaced with histidine (H487). This change replaces asparagine, a neutral amino acid with a polar side chain, with a residue that also carries a polar side chain, however, one that is charged below pH 6. Histidine can be considered strongly similar in properties to asparagine based on scoring in the PAM 250 matrix. Evolution has resulted in a similar change at this site for the HSP70 variant known as GRP75 in rabbits (FIG. 1) and humans (FIG. 2).

Change to D-P Site: In some embodiments, proline-81 (P81) in HSP72 is replaced with alanine (A81). The presence of a proline residue following an aspartic acid can result in an acid labile peptide bond that can be cleaved under low pH conditions [Marcus, F. (1985) Preferential cleavage at aspartyl-prolyl peptide bonds in dilute acid. Int J Pept. Protein Res. 25 (5): 542-546]. By changing from an aspartic acid-80 (D80)-P81 sequence to a D80-A81 configuration, a cleavage site is removed and product stability improved at low pH. This change replaces proline with alanine, both non-polar amino acids. However, due to significant structural differences in the proline and alanine side chains, this replacement can be weakly similar in properties according to the PAM 250 matrix.

In some embodiments, in Change(s) to N-P Sites: The presence of a proline residue following an asparagine can result in an alkaline-labile peptide bond that can be cleaved under high pH conditions, while the presence of a leucine, serine or threonine following an asparagine may result in partial cleavage under high pH conditions [Tarelli, E., and Conan, P. H. 2003. Ammonia cleaves polypeptides at asparagine proline bonds. J. Pept. Res. 62 (6): 245-251]. In some embodiments, the asparagine (N) is replaced with glutamine (Q), an amino acid with weakly similar properties due to its larger side chain, or with serine (S), an amino acid with strongly similar properties to asparagine, both according to the PAM 250 matrix. In some embodiments, the proline (P), leucine (L), serine (S) or threonine (T) following an asparagine is replaced with one of those amino acids listed in the Aliphatic (except leucine), Aromatic, Basic or Acidic and their Amide Groups. By eliminating alkaline-labile sequences, particularly N-P sequences that are completely cleaved under high pH conditions, product stability is improved at high pH.

In some embodiments, when modifying HSP, one or more sites among the glycosylation sites, acid-labile sites and alkaline-labile sites can be modified. In some embodiments, a modified HSP has one or more or all glycosylation sites modified while none of acid-labile and alkaline-labile cleavage sites, if present in the HSP protein, is modified. In some embodiments, a modified HSP has one or more or all acid-labile cleavage sites modified while none of glycosylation sites and none of alkaline-labile cleavage sites, if present in the HSP protein, is modified. In some embodiments, a modified HSP has one or more or all alkaline-labile cleavage sites modified while none of glycosylation sites and none of acid-labile cleavage sites, if present in the HSP protein, is modified. In some embodiments, when modifying HSP, one or more or all glycosylation sites and one or more or all acid-labile cleavage sites are modified while none of alkaline-labile cleavage sites, if present in the HSP protein, is modified. In some embodiments, when modifying HSP, one or more or all glycosylation sites and one or more or all alkaline-labile cleavage sites are modified while none of acid-labile cleavage sites, if present in the HSP protein, is modified. In some embodiments, when modifying HSP, one or more or all acid-labile cleavage sites and one or more or all alkaline-labile cleavage sites are modified while none of glycosylation sites, if present in the HSP protein, is modified. In some embodiments, when modifying HSP, one or more or all glycosylation sites, one or more or all acid-labile cleavage sites and one or more or all alkaline-labile cleavage sites are modified. In some embodiments, a modified HSP has all glycosylation sites, all acid-labile cleavage sites and all alkaline-labile cleavage site modified. In some embodiments, e.g. in an HSP72 modification, all 5 glycosylation sites is modified along with the D-P cleavage site. It is also contemplated to not modifying the D-P site while still modifying all 5 glycosylation sites. It is also contemplated to modifying only 1, 2, 3, 4 or 5 glycosylation sites with or without modifying the D-P site, e.g. if one may find that some N-X-S/T/C sites can be less amenable to glycosylation in one eukaryotic production system versus another. Similarly, in some embodiments, e.g. in an HSP72 modification, all alkaline-labile sites can be modified or one can consider modifying only 1, 2, 3, 4, 5, 6 or 7 alkaline-labile sites along with all or some of the glycosylation sites and with or without modification to the D-P acid-labile cleavage site.

In some embodiments, when modifying HSP, about 10%, about 20%, about 30%, about 40%, about 50%, about 60%, about 70%, about 80%, about 90%, about 95%, about 99%, or about 100% of the total number of the N-glycosylation sites is modified and about 10%, about 20%, about 30%, about 40%, about 50%, about 60%, about 70%, about 80%, about 90%, about 95%, about 99%, or about 100% of the total number of the acid-labile sites is modified and about 10%, about 20%, about 30%, about 40%, about 50%, about 60%, about 70%, about 80%, about 90%, about 95%, about 99%, or about 100% of the total number of the alkaline-labile sites is modified.

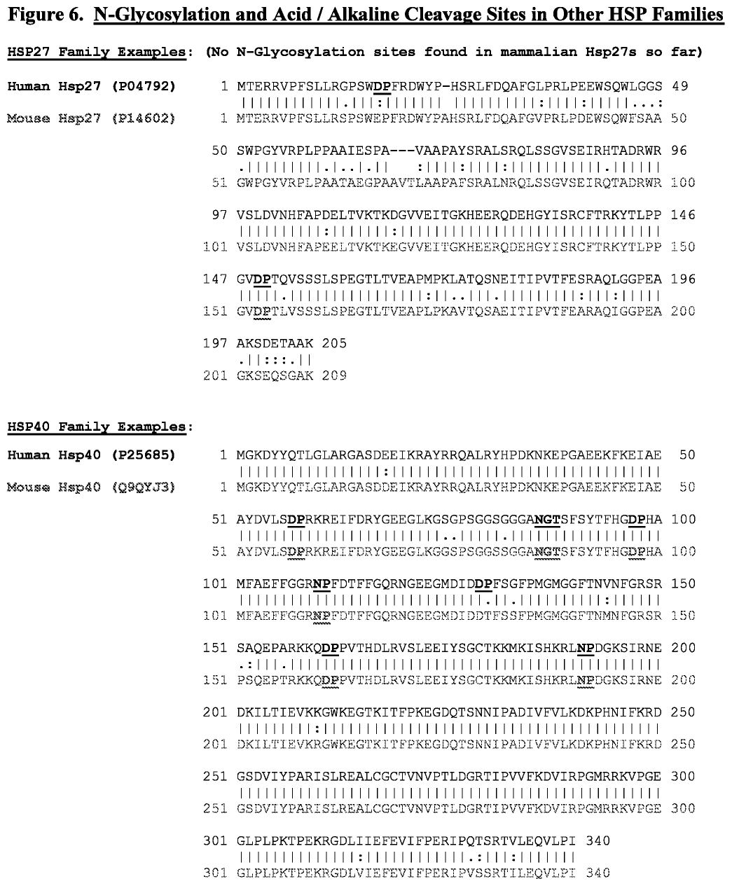

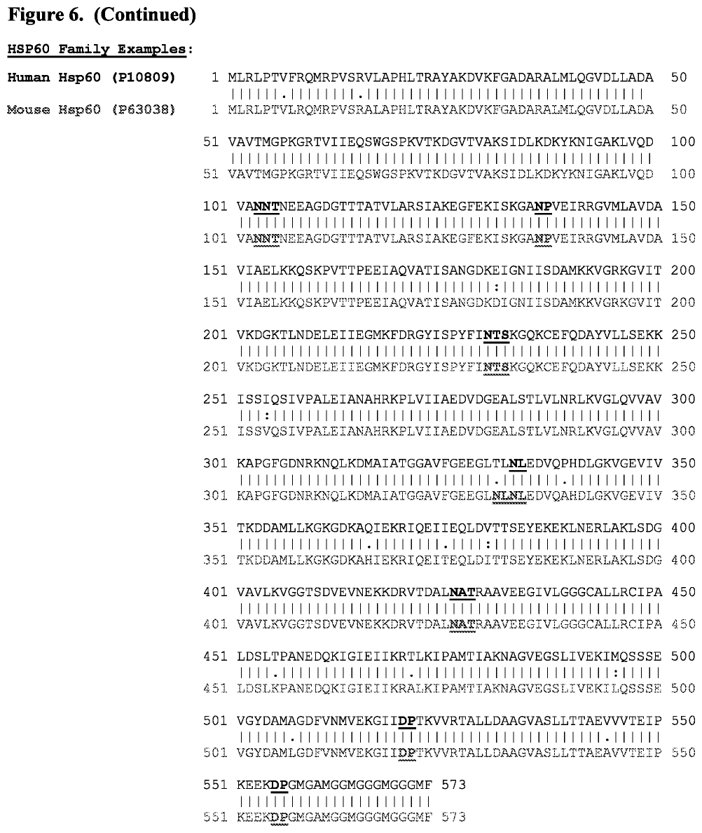

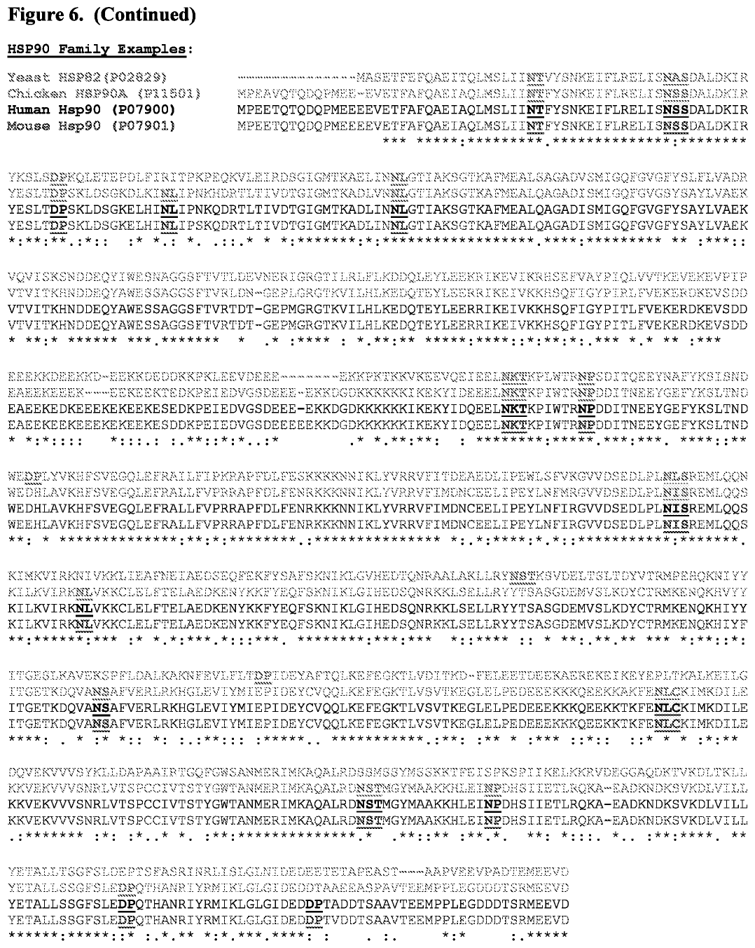

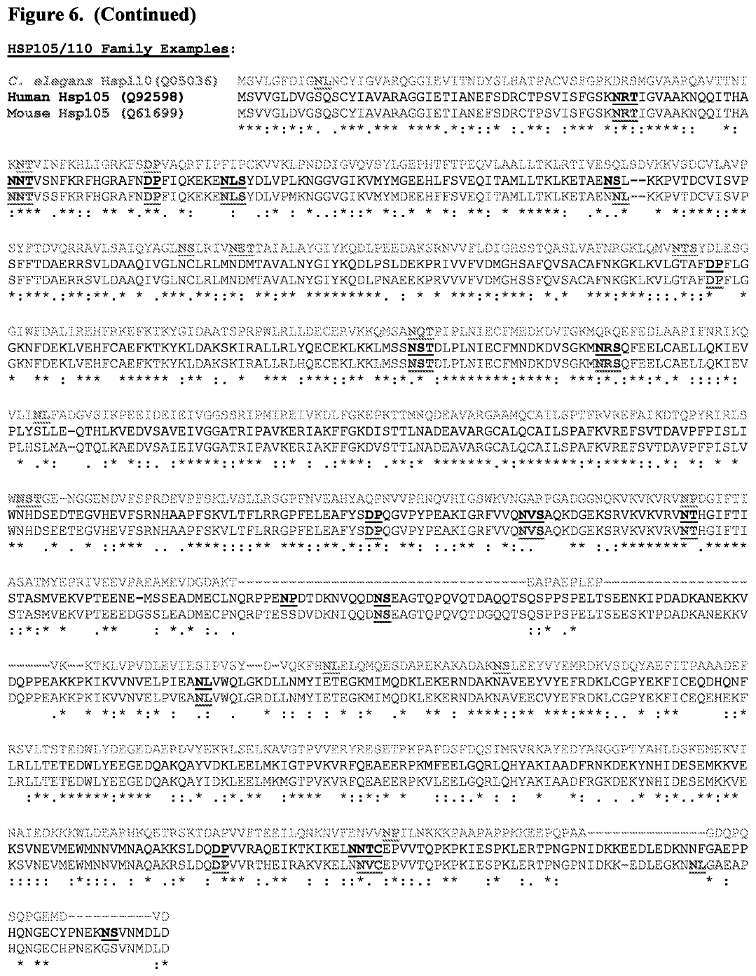

Some embodiments of the disclosure relate to the modification of other heat shock proteins. Successful elimination of glycosylation sites and an acid-labile cleavage site from HSP72 to create a new functional member of the HSP70 family of proteins proves similar changes can be made to other classes of heat shock proteins. A survey of several HSP families finds the following (FIG. 6):

HSP27 Family: No N-X-S/T/C sites in mammals. Two D-P sites in human HSP27. No putative alkaline-labile sites in mammals.

HSP40 Family: One N-X-S/T/C sites in mammals. Four D-P sites in human HSP40. Two putative alkaline-labile sites in mammals.

HSP60 Family: Three N-X-S/T/C sites in mammals. Two D-P sites in mammalian HSP60. Four putative alkaline-labile sites in humans.

HSP90 Family: Four N-X-S/T/C sites, five putative alkaline-labile sites and three D-P sites in yeast. Five N-X-S/T/C sites in mammals and birds. Ten putative alkaline-labile sites in mammals and birds. Two D-P sites in birds and three D-P sites in mammals.

HSP105/110 Family: Four N-X-S/T/C sites, ten putative alkaline-labile sites and one D-P site in nematodes. At least seven N-X-S/T/C sites, at least seven putative alkaline-labile sites and four D-P sites in mammals.

In some embodiments, modifications of one or more of these N-X-S/T/C sites create new sequences that produce improved heat shock proteins for commercial production in multiple cellular systems, including, but not limited to, bacterial, fungal, insect, and mammalian cell lines, without concern for N-linked glycosylation.

In some embodiments, modifications to one or more of the D-P sites create new sequences that produce improved heat shock proteins that are more stable in moderately acidic buffers and formulations. In some embodiments, modifications to one or more of the N-P, N-L, N-S or N-T sites create new sequences that produce improved heat shock proteins that are more stable in moderately alkaline buffers and formulations.

In a non-limiting example, a modified human HSP27 that has both acid labile D-P cleavage sites removed may prove stable enough to be used for the treatment of retinal injuries by injecting the modified HSP27 intravitreally into the ocular globe. In some embodiments, the small molecular weight of HSP27 and greater stability can allow longer residency within the vitreous and provide greater efficacy in treating retinal injuries.

In another non-limiting example, a modified human HSP105/110 that has its eight putative N-linked glycosylation sites eliminated may be used in conjunction with HSP72 in the treatment of brain injuries. HSP110 has been identified as a co-factor of HSP72 in some anti-apoptotic processes in neural cells. A glycosylated version of the HSP110 could prove ineffective if the carbohydrate structures were to interfere in the HSP110's interactions with HSP72. In some embodiments, the modified HSP110 can allow more efficient interaction with HSP72 and provide greater efficacy in treating brain injuries.

In yet another non-limiting example, a modified human HSP60 that has its three putative N-glycosylation sites and four putative alkaline-labile sites eliminated can be manufactured in a fungal system, such as Pichia or Saccharomyces, where ammonium sulfate and ammonium hydroxide are used during pH control of the fermentation process. In human HSP60, two N-T sites are embedded within putative N-glycosylation sites (FIG. 6), hence modifications that eliminate both N-glycosylation and alkaline pH cleavage of proteins can be applied simultaneously.

In some embodiments, the composition includes one type of modified HSP or a plurality of different types of HSPs. Thus, in some embodiments, the composition includes modified HSP72 but does not include other modified HSPs. In some embodiments, the composition can include the combination of one or more modified HSPs and one or more wild-type HSPs. The HSPs do not have to be from the same species. In some embodiments, the composition can include modified HSP72 and one or more other types of modified HSPs, e.g. the combination of modified HSP72 and modified HSP110. Alternatively, the composition can include the combination of modified HSP72 and wild-type HSP110. Any combinations having two or more different types of HSPs can be included in the composition and used for treatment in various embodiments of the disclosure. In some embodiments, the combinations can include an HSP70 with an HSP110, an HSP70 with an HSP40, an HSP70 with an HSP27, and HSP110 with an HSP27, one of the small heat shock proteins (HSP10 or HSP17) with an HSP60, where one or both constituents may be modified according to the teachings in this patent. In some embodiments, the combinations can include an HSP70, an HSP40 and an HSP110 as one composition; or an HSP27, an HSP70 and an HSP110 as one composition. In some embodiments, the HSPs can be administered in combination as separate molecules. In some embodiments, the HSPs can be fused in tandem and administered as a single fusion molecule.

In some embodiments, heat shock proteins in accordance with the present disclosure can be a full sequence of wild-type, naturally occurring HSP, any functional derivative or functional fragment thereof, e.g. a derivative or fragment having the same or similar activities and utilities as the corresponding wild-type protein.

One skilled in the art will appreciate that the complete amino acid sequence of any one of the modified HSP polypeptides as disclosed herein can be used to construct a back-translated gene. For example, a DNA oligomer containing a nucleotide sequence coding for a given polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the DNA sequences encoding a modified HSP polypeptide as disclosed herein will be inserted into an expression vector and operably linked to an expression control sequence appropriate for expression of the modified HSP polypeptides as disclosed herein in the desired transformed host. Proper assembly can be confirmed by nucleotide sequencing, restriction mapping, and expression of a biologically active polypeptide in a suitable host. As is known in the art, in order to obtain high expression levels of a transfected gene in a host, the gene must be operably linked to transcriptional and translational expression control sequences that are functional in the chosen expression host, e.g., fungi, insects, or mammals.

In addition or alternatively, the generation of any one of the modified polypeptides described herein can be achieved via expression of nucleic acid molecules that have been altered by recombinant molecular biological techniques. Furthermore, the modified polypeptides in accordance with the present disclosure can be chemically synthesized. Chemically synthesized polypeptides are routinely generated by those of skill in the art.

### Production of Modified Heat Shock Proteins (HSPs) and Conjugates or Fusions Thereof.

Some embodiments disclosed herein relate to a method for preparing modified heat shock proteins (HSPs)..

HSP72 has an ATP binding domain and a substrate binding domain. In some embodiments, the ATP binding domain can be partially or entirely eliminated from the methionine at residue 1 through the glutamine at residue 389 such that the construct for HSP72 only consists of the substrate binding domain from the aspartic acid at residue 390 through the aspartic acid at residue 641. Such a construct only has 2 glycosylation sites and no D-P site and in some embodiments, the 2 glycosylation sites can be modified.

In another aspect, some embodiments of the disclosure relate to a method for preparing a conjugate or fusion protein as described herein using methods known in the art. Some embodiments provide a method for transporting the modified HSP into the cell by conjugation or fusion of proteins, protein fragments, peptide leader sequences and poly-cation sequences that target nucleic acids. In some embodiments, a method for transporting the modified HSP into the cells utilizes the 3E10 antibody or a fragment thereof, such as a single chain Fv (scFv). The 3E10 antibody can be the original murine sequence, a humanized sequence, a fully human analog or one that is modified by specific amino acid changes to the CDRs or framework of the antibody to either improve binding or reduce immunogenicity.

In some embodiments, methods of transport of modified HSPs across the cell membrane include, but are not limited to, conjugation or fusion of proteins or peptides that bind nucleic acids, such as histones, histone tails, high mobility group proteins (HMGs), transcription factors or poly-cation sequences of the amino acids lysine and/or arginine. In some embodiments, they can include conjugation or fusion of antibodies or antibody fragments that target DNA, RNA, nucleic acids in general, histones, HMGs or transcription factors. In some embodiments, they can also include direct attachment, either by genetic means or by chemical conjugation, of the modified HSP to one or more nucleic acids or oligonucleotide sequences. In some embodiments, the antibody or fragment thereof binds to a molecule (e.g. nucleic acids such as DNA and RNA) present inside a cell. In some embodiments, the antibody or fragment thereof binds to a molecule (e.g. nucleic acids such as DNA and RNA) present outside a cell. Thus, in some embodiments, a 3E10 antibody binds extracellular DNA and nucleotides before being transported into the cell across the plasma membrane via the ENT channel.

In some embodiments, methods of transport of modified HSPs across the cell membrane include phospholipid binding proteins or fragments thereof. Such proteins include, but are not limited to, annexins, lactadherin, sphingomyelin, apolipoprotein-H (a.k.a. β-2-glycoprotein-1), T-cell immunoglobulin mucin domain (TIM) receptors and galectins. In some embodiments, they also include specific cell-penetrating proteins and peptides, such as TAT proteins from the HIV virus or Antennapedia proteins from insects, to name just a few. In some embodiments, they also include binding of ions transported across the membrane, such as binding of calcium and calcium ions transported through channels. In some embodiments, they can also include antibodies or antibody fragments targeting the proteins listed above.

In some embodiments, once transported, the linkage between modified HSP and its conjugating or fusion partner (e.g. a cellular transporting molecule) can remain intact in the intracellular environment, or can be cleaved by the engineering of specific cleavage sites within the primary structure of the molecule. For example, an esterase cleavage site can be present between HSP and the targeting (or transporting) molecule, which transports the HSP across a lipid bilayer, e.g. a cell membrane. Therefore, enzymatic cleavage by an esterase may separate the two components once they are delivered in the cell.

In some embodiments, the pharmaceutical composition contains is formulated into a liposome In some embodiments, cellular internalization can also be achieved by placing the modified HSP72 or any modified HSP into a liposome coated with any of the aforementioned molecules for fusion with the cell membrane. In some embodiments, the liposome can be coated with 3E10 antibody or a fragment thereof In some embodiment, the liposome can contain a combination of the modified HSP72 and/or other modified HSPs. In some embodiment, the liposome can contain a combination of modified HSPs and wild-type HSPs.

In some embodiments, the composition includes one or more modified HSPs as well as conjugates and/or fusions thereof. In some embodiments, the composition includes one or more modified HSPs but not conjugates and/or fusions thereof In some embodiments, the composition includes conjugates and/or fusions of modified HSPs but not modified HSPs. Treatment Methods

Some embodiments of the disclosure relate to the use of modified heat shock proteins or HSPs to treat a disease or condition in a subject who is in need of such treatment. In some embodiments, the method includes administering to the subject an effective amount of a polypeptide or a composition as disclosed herein. In some embodiments, such target disease or condition include, but not limited to, ocular, neurological, muscular, hepatic, renal, integumentary, cardiovascular and pulmonary conditions and traumas.

In some embodiments of the disclosure, the treatment methods include administering an effective amount of a polypeptide or a composition as disclosed herein, which contains one or more modified HSPs, fragments thereof, conjugate and/or fusion protein thereof. In some embodiments, the methods include intravenous (IV), intravitreal (IVT), intranasal (IN), intramuscular (IM) and subcutaneous (SQ) injection. In some embodiments, the methods include inhalation as an aerosol mist or dry powder. In some embodiments, the methods include topical application to the surface of the skin or eyes, particularly in regions that are wounded.

In some embodiments where ocular injuries or diseases are concerned, the composition can be administered by IVT, IV, IN or by topical application to the surface of the eye to patients suffering from a variety of ocular injuries and traumas, including but not limited to, central retinal artery occlusion (CRAO), macular degeneration (either wet or dry), Stargardt disease (fundus flavimaculatus) and Retinitis pigmentosa (RP), and other retinal maladies. In the case of CRAO, the composition can be administered to counter the damaging effects on cells from hypoxia during the occlusion and counter the damaging effects on cells from oxidative stress during reperfusion. The composition can also be administered to inhibit cellular damage caused by glaucoma. The composition can be administered to inhibit damage caused by physical or other pressure injuries, including, but not limited to, physical impacts and blast injuries. The composition can also be administered to inhibit cell death and improve tissue recovery after trauma to the cornea and surrounding ocular surface, including but not limited to, corneal damage caused by chemical exposure or abrasions. The composition can also be administered to help improve tissue recovery after corneal transplantation, ocular punctures or incisions as part of a surgical procedure, such as cataract surgery. The composition can be administered as a single dose or repeatedly over an extended period of time.

In some embodiments where cardiovascular injuries or diseases are concerned, the composition can be administered by IV, IM, SQ, by inhalation or topical application (either through the skin or directly to the surface of the tissue during surgery), to counter the damaging effects of a cardiovascular occlusion or embolism on cells from hypoxia during the occlusion and counter the damaging effects on cells from oxidative stress during reperfusion. The composition can also be administered to inhibit cell death caused by acute or chronic conditions of heart failure. The composition can be administered as a single dose or repeatedly over an extended period of time. In some embodiments, it can be injected intravascularly (intra-venous or intra-arterial) or it can be injected directly to the heart using a catheter, for example after a stent has been placed, or it can be administered using a combination of both methods to a patient as part of long term care.

In some embodiments, where neurological injuries or diseases are concerned, the composition can be administered by IV, IVT, IN, IM, SQ, by inhalation or topical application (either through the skin or directly to the surface of the tissue during surgery), to counter the damaging effects of a neurological or neurovascular occlusion or embolism (stroke) on cells from hypoxia during the occlusion and counter the damaging effects on cells from oxidative stress during reperfusion. The composition can also be administered to inhibit cell death caused by acute or chronic conditions such as the aftermath of traumatic brain injury to inhibit damage caused by physical or other pressure injuries, including, but not limited to, physical impacts and blast injuries. The composition can also be administered to inhibit cell death caused by epilepsy and epileptic seizures, including status epilepticus. The composition can be used to inhibit cell death caused by plaques and denatured protein conglomerations characteristic of Alzheimer's and other dementias. The composition may be used to renature proteins found in such plaques to help clear them from neural tissues. The composition can be used to inhibit cell death caused by multiple sclerosis (MS). The composition can be administered to inhibit cell death caused by exposure to neurotoxins, either chemical or biological in nature, including but not limited to, organophosphates, paraoxon and other organophosphate oxons, Tabun (GA), Sarin (GB), Soman (GD), Cyclosarin (GF), GV, EA-3148, VE, VG, VM, VR, VX, Novichok agents, Agent 15, dimethylheptylpyran, EA-3146, Kolokol-1, PAVA spray, pepper spray, CS, mace (CN), CR, neurological venoms, etcetera. In embodiments, the composition can be administered to inhibit cell death and neural damage caused by anesthesia exposure, particularly in elderly patients. The composition can be administered to inhibit cell death caused by diabetes, including but not limited to neural damage caused by the disease. The composition can be administered as a single dose or repeatedly over an extended period of time. In some embodiments, the composition can be injected intravascularly or it can be injected directly to the neurovasculature through the carotid artery using a catheter, for example after a stent has been placed, or it can be administered using a combination of both methods to a patient as part of long term care.

In some embodiments where pulmonary injuries or diseases are concerned, the composition can be administered by IV, IN, IM, SQ, by inhalation or topical application (either through the skin or directly to the surface of the tissue during surgery), to counter the damaging effects of traumas. The composition can be administered to counter the damaging effects of a pulmonary occlusion or embolism on cells from hypoxia during the occlusion and counter the damaging effects on cells from oxidative stress during reperfusion. The composition can also be administered to inhibit cell death caused by acute or chronic conditions of pulmonary failure, including but not limited to, chronic obstructive pulmonary disease (COPD), emphysema, chronic bronchitis, refractory (non-reversible) asthma, reversible asthma and bronchiectasis. The composition can be administered to counter tissue damage caused by inhaled toxins, including but not limited to, phosgene (CG), phosgene oxime (CX), diphosgene (D-P), triphosgene (TP), chlorine, chloropicrin (PS), bromine, disulfur decafluoride, cyanide and smoke. For inhalational administration, the composition can be delivered to any area of the upper or lower lung passageways either as an aerosol mist or dry powder using inhalational devices known to those well-versed in the art. The composition can be administered to the patient as a single dose or repeatedly over an extended period of time.

In some embodiments where muscular injuries or diseases are concerned, the composition can be administered by IV, IM, SQ, by inhalation or topical application (either through the skin or directly to the surface of the tissue during surgery), to counter the damaging effects of traumas, such as neuromuscular damage caused by physical injuries, muscular dystrophy, fibromyalgia, and chronic muscle pain. The composition can be used to treat damage caused by exposure to venoms or chemical toxins, acids, bases, etcetera. The composition can also be used to treat muscular abnormalities, such as glycogen storage diseases (GSDs), for example, Pompe's disease (GSD Type II), Forbes-Cori disease (GSD Type III), McArdle's disease (GSD Type V), Tarui's disease (GSD Type VII), Red cell aldolase deficiency (GSD Type XII), β-enolase deficiency (GSD Type XIII) and glycogenin-1 deficiency (GSD Type XV). The composition can also be used to treat muscle weakness or atrophy caused by centronuclear myopathies (CNM), including but not limited to X-linked myotubular myopathy, BIN1-related CNM, DNM2-related CNM, RYR1-related CNM, etcetera. The composition can be administered to the patient as a single dose or repeatedly over an extended period of time.

In some embodiments, where hepatic injuries or diseases are concerned, the composition can be administered by IV, IM, SQ, by inhalation or topical application (either through the skin or directly to the surface of the tissue during surgery), to counter the damaging effects of cirrhosis regardless of the underlying causes which include, but are not limited to, alcoholism, acetaminophen or other drug overdose, microbial or viral infection. The composition can also be used to treat the hepatic symptoms of metabolic disorders, such as glycogen storage diseases (GSDs), for example Andersen's disease (GSD Type IV), which can lead to hepatomegaly and cirrhosis. The composition can be administered to the patient as a single dose or repeatedly over an extended period of time.

In some embodiments where renal injuries or diseases are concerned, the composition can be administered by IV, IM, SQ, by inhalation or topical application (either through the skin or directly to the surface of the tissue during surgery), to counter the damaging effects of kidney failure. The composition can be used to treat such traumatic injuries as rhabdomyolysis and Bywaters' syndrome (also known as traumatic rhabdomyolysis or “crush syndrome”). Damage to the skeletal musculature, particularly in a traumatic event involving a physical impact (e.g. a car accident, falling masonry from a building collapse, etcetera), can lead to the release into the bloodstream of muscle breakdown products as muscle tissue dies, including myoglobin, potassium and phosphorus (e.g. the breakdown products seen with rhabdomyolysis). These breakdown products can lead to nephrotoxicity and subsequent kidney failure. In some embodiments, administration of the composition can reduce cellular stress and inhibit apoptosis of renal cells and the renal vasculature. The composition can also be used to treat kidney damage caused by auto-immune diseases, including but not limited to, systemic lupus erythematosus (SLE), anti-phospholipid syndromes, and anti-nuclear antibodies (e.g. anti-Ro antibodies, anti-La antibodies, anti-Sm antibodies, anti-nRNP antibodies, anti-Scl-70 antibodies, anti-dsDNA antibodies, anti-histone antibodies, antibodies to nuclear pore complexes, anti-centromere antibodies and anti-sp100 antibodies). The composition can be administered to the patient as a single dose or repeatedly over an extended period of time.

For integumentary wounds caused by physical or chemical traumas, including abrasions, burn wounds, venoms, exposure to chemicals, exposure to blistering agents (e.g. sulfur mustard in any of its forms: H, HD, HT, HL, or HQ; nitrogen mustard in any of its forms: HN1, HN2, or HN3; Lewisite [L], ethyldichloroarsine [ED], methyldichloroarsine [MD], phenyldichloroarsine [PD], etcetera) or other toxic wounding, the composition can be administered by IV, IN, IM, SQ, by inhalation or topical application (either through the skin or directly to the surface of the tissue during surgery), to counter the damaging effects. The composition can be administered to the patient as a single dose or repeatedly over an extended period of time.

In some embodiments where oncological diseases are concerned, the composition can be used to ameliorate the apoptotic effects of cancer therapies on normal tissues away from the tumor microenvironment. For example, cardiotoxicity is a major side effect of some cancer therapies. Patients undergoing treatment can have the composition provided directly to or near the region of the heart to counter apoptosis of cardiac cells. In some cases, another challenge is the effect of chemotherapy on the cognitive and memory processes (colloquially referred to as “chemo brain”). Patients undergoing treatment can have the composition provided directly to or near the region of the brain to counter apoptosis of neural cells. The composition can be administered by IV, IVT, IN, IM, SQ, by inhalation or topical application (either through the skin or directly to the surface of the tissue during surgery), to counter the damaging effects. The composition can be administered to the patient as a single dose or repeatedly over an extended period of time.

For organ transplantation, the composition can be used to counter apoptosis of cells immediately after transplantation, to counter the effects of transplant rejection from the host (e.g. liver transplantation), or to counter the effects of graft versus host disease (GVHD) caused by the grafted tissue (e.g. in bone marrow transplants). Rejection can affect the organ being transplanted or other tissues in the body through indirect mechanisms, such as inflammation. The composition can be administered by IV, IVT, IN, IM, SQ, by inhalation or topical application (either through the skin or directly to the surface of the tissue during surgery), to counter the damaging effects. The composition can be administered to the patient as a single dose or repeatedly over an extended period of time.

For regenerative medicine, the composition can be used to supplement the activation of stem cells or transplantation of regenerative tissues grown ex vivo. The composition can be administered by IV, IVT, IN, IM, SQ, by inhalation or topical application (either through the skin or directly to the surface of the tissue during surgery), to help maintain or improve recovery of tissues, including but not limited to, spinal cord injuries, skin grafts, corneal transplants, etcetera. The composition can be administered to the patient as a single dose or repeatedly over an extended period of time.

In some embodiments, the composition can also be added to culture media for cells in vitro to minimize loss of viability caused by stressful conditions. In some embodiments, the composition can be added to media used in recovering tissue samples from a donor to minimize loss of viability during the process. In some embodiments, the composition can be added to media during the generation and propagation of stem cells to improve viability of the cells during their culture. In some embodiments, the composition can be administered to the media as a single dose or repeatedly over an extended period of time.

The polypeptides of the disclosure, including the modified HSPs and fragments thereof as described herein, can be used to treat patients who have, who are suspected of having, or who may be at high risk for developing one or more health conditions or disorders. Exemplary disorders and health conditions can include, without limitation, ocular, neurological, muscular, hepatic, renal, integumentary, cardiovascular, and pulmonary conditions and traumas. In one aspect, some embodiments of the disclosure relate to a method of treating cells ex vivo or in vitro. The method includes adding an effective amount of any of the modified heat shock protein or fragment thereof disclosed herein to a culture media. In some embodiments, the method maintains the viability of the cells during their culture and propagation.

### Formulation

In some embodiments of the disclosure, the pharmaceutical composition is formulated to be compatible with its intended route of administration. Accordingly, some embodiments of the disclosure relate to formulations, e.g. pharmaceutical compositions that include any of the modified HSPs described herein, or any of the conjugates, fusion proteins or derivatives thereof, and a pharmaceutically acceptable excipient, e.g. pharmaceutically acceptable carrier. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics) can also be incorporated into the compositions.

Pharmaceutical compositions of the present disclosure containing modified HSPs or conjugate, fusion or derivative thereof as an active ingredient may contain pharmaceutically acceptable excipients or additives depending on the route of administration. Parenteral administration means any non-oral means of administration, and is generally interpreted by those skilled in the art as relating to direct injection into the body, bypassing the skin and mucous membranes. Common parenteral routes of administration are intramuscular (IM), subcutaneous (SC), and intravenous (IV). See, e.g., www.nursingtimes.net/administration-of-drugs-3-parenteral/5034777.article. An appropriate composition having the active ingredient(s) to be administered can be prepared in a physiologically acceptable vehicle or carrier. For solutions or emulsions, suitable carriers include, for example, aqueous or alcoholic/aqueous solutions, emulsions or suspensions, including saline and buffered media.

In some embodiments, the composition as disclosed herein, which contains modified HSPs or conjugate, fusion or derivative thereof, can be formulated as a liquid for parenteral delivery, or as a lyophilized product for rehydration prior to administration. In some embodiments, in its final liquid formulation, the composition can be buffered for application to the blood, vitreous or other bodily fluids. In some embodiments, the pharmaceutical composition contains is formulated into a liposome. Strategies, methodologies, and techniques suitable for preparation of stable liposomal formulations comprising one or more therapeutic polypeptides are known in the art. For example, Liposomal suspensions can also be used as pharmaceutically acceptable carriers. These can be prepared according to methods known to those skilled in the art, for example, as described in U.S. Pat. No. 4,522,811. In some embodiments, the modified proteins and pharmaceutical compositions of the disclosure can also be buffered for application to the surface of the eye.

For delivery to the lungs or nasal passages, the composition can be formulated as an aerosol mist for delivery with a device known to those well-versed in the art. For delivery to the lungs, the composition can be formulated as a dry powder for delivery with a device known to those well-versed in the art.

For topical application to the skin, the composition can be formulated into an ointment.

In some embodiments, an effective amount of the polypeptides and compositions in accordance with the present disclosure or active ingredient thereof, e.g. modified HSPs and/or conjugates or fusion proteins thereof, is administered to an individual in need thereof For example, in some embodiments, the composition can reduce cell death when the composition or active ingredient thereof is administered in an effective amount. The amount administered varies depending upon the goal of the administration, the health and physical condition of the individual to be treated, age, the taxonomic group of the individual to be treated (e.g., human, non-human primate, primate, etc.), the degree of resolution desired, the formulation of the bispecific antibody or composition, the treating clinician's assessment of the medical situation, and other relevant factors. It is expected that the amount will fall in a relatively broad range that can be determined through routine trials. For example, the amount of the composition or active ingredient thereof employed to reduce cell death is not more than about the amount that could otherwise be irreversibly toxic to the subject (e.g., maximum tolerated dose).

In some embodiments, the polypeptides and/or compositions of the disclosure are prepared with carriers that will protect the polypeptides and/or compositions of the disclosure against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to specific cells or tissues) can also be used as pharmaceutically acceptable carriers. These can be prepared according to methods known to those skilled in the art, for example, as described in U.S. Pat. No. 4,522,811.

Dosage, toxicity and therapeutic efficacy of the polypeptides and compositions of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC50 (e.g, the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a “therapeutically effective amount” of a subject polypeptide of the disclosure (e.g, an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU) as established by the World Health Organization 1st International Standard for Interleukin-2 (human)). The dosage may be similar to, but is expected to be less than, that prescribed for PROLEUKIN®. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the subject polypeptide of the disclosure, e.g., a modified HSP, a fragment thereof, and/or conjugates or fusion proteins thereof, can include a single treatment or, can include a series of treatments. In one embodiment, the compositions are administered every 8 hours for five days, followed by a rest period of 2 to 14 days, e.g., 9 days, followed by an additional five days of administration every 8 hours.

In some embodiments, the composition or active ingredient thereof can be administered in a dose (or an amount) of about 1 ng/kg of subject body weight, about 10 ng/kg of subject body weight, about 50 ng/kg of subject body weight, about 100 ng/kg of subject body weight, about 500 ng/kg of subject body weight, about 1 μg/kg of subject body weight, about 10 μg/kg of subject body weight, about 50 ug/kg of subject body weight, about 100 μg/kg of subject body weight, about 150 μg/kg of subject body weight, about 200 μg/kg of subject body weight, about 250 μg/kg of subject body weight, about 300 μg/kg of subject body weight, about 350 μg/kg of subject body weight, about 375 μg/kg of subject body weight, about 400 μg/kg of subject body weight, about 450 μg/kg of subject body weight, about 500 μg/kg of subject body weight, about 550 μg/kg of subject body weight, about 600 μg/kg of subject body weight, about 650 μg/kg of subject body weight, about 700 μg/kg of subject body weight, about 750 μg/kg of subject body weight, about 800 μg/kg of subject body weight, about 850 μg/kg of subject body weight, about 900 μg/kg of subject body weight, about 1 mg/kg of subject body weight, about 10 mg/kg of subject body weight, about 50 mg/kg of subject body weight, about 100 mg/kg of subject body weight, about 500 mg/kg of subject body weight, about 1 g/kg of subject body weight or more or any intervening ranges of the of the foregoing. In some embodiments, the composition or active ingredient thereof can be administered in a dose (or an amount) of about 0.5 μg, about 1.0 μg, about 1.5 μg, about 2.0 μg, about 2.5 μg, about 3.0 μg, about 3.5 μg, about 4.0 μg, about 4.5 μg about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 1.0 mg, about 1.5 mg, about 2.0 mg, about 2.5 mg, about 3.0 mg, about 3.5 mg, about 4.0 mg, about 4.5 mg about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, about 1 g or more or any intervening ranges of the foregoing.

In some embodiments, administration of the composition or active ingredient thereof can reduce cell death. The reduction of cell death caused by this administration includes an about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, or about 99% reduction of cell death as compared to no administration of the composition or active ingredient thereof. In some embodiments, the cells to which the composition or active ingredient thereof is administrated are stressed or injured cells that have a risk of undergoing cell death. In some embodiments, such stressed or injured cells can include cells that are present or obtained from a subject suffering from one or more of ocular, neurological, muscular, hepatic, renal, integumentary, cardiovascular and pulmonary conditions and traumas.

Administration of the composition or active ingredient thereof can be repeated over a desired period, e.g., repeated over a period of about 1 day to about 5 days or once every several days, for example, about five days, over about 1 month, about 2 months, etc. It also can be administered prior, at the time of, or after other therapeutic interventions, such as surgical intervention to remove damaged or pathogenic cells. The composition can also be administered as part of a combination therapy, in which at least one of an immunotherapy, a cancer chemotherapy or a radiation therapy is administered to the subject.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

Additional embodiments are disclosed in further detail in the following examples, which are provided by way of illustration and are not in any way intended to limit the scope of this disclosure or the claims.

### General Experimental Procedures

The practice of the present disclosure will employ, unless otherwise indicated, conventional techniques of molecular biology, microbiology, cell biology, biochemistry, nucleic acid chemistry, and immunology, which are known to those skilled in the art. Such techniques are explained in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F.M. Ausubel et al., eds., 1987, including supplements through 2014); PCR: The Polymerase Chain Reaction, (Mullis et al., eds., 1994); Beaucage et al. eds., Current Protocols in Nucleic Acid Chemistry, John Wiley & Sons, Inc., New York, 2000, (including supplements through 2014), Gene Transfer and. Expression in Mammalian Cells (Makrides, ed., Elsevier Sciences B. V., Amsterdam, 2003), and Current Protocols in Immunology (Horgan K and S. Shaw (1994) (including supplements through 2014). As appropriate, procedures involving the use of commercially available kits and reagents are generally carried out in accordance with manufacturer defined protocols and/or parameters unless otherwise noted.

### Example 1: Efficacy and Stability Testing of Compositions

Product characterization involves testing for degradation using SDS-PAGE, aggregation using size exclusion chromatography (SEC), product identity using mass spectrometry and western blotting. These are standard techniques. Testing product efficacy is often product specific.

To test the functionality of the modified HSP72, it was necessary to transport it into the cells, hence the 3E10 antibody was used in this particular example. Testing could have been conducted by fusing the HSP72 to other internalizing proteins, including but not limited to, histones, annexins, cell penetrating peptides, phosphatidyl serine binding proteins and direct binding to nucleic acids themselves. Internalization can also be achieved by placing the modified HSP72 into a liposome coated with any of the aforementioned proteins for fusion with the cell membrane.

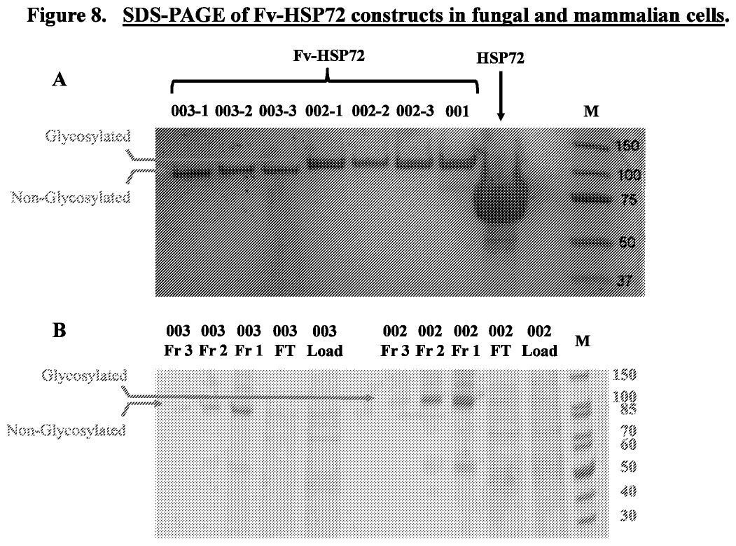

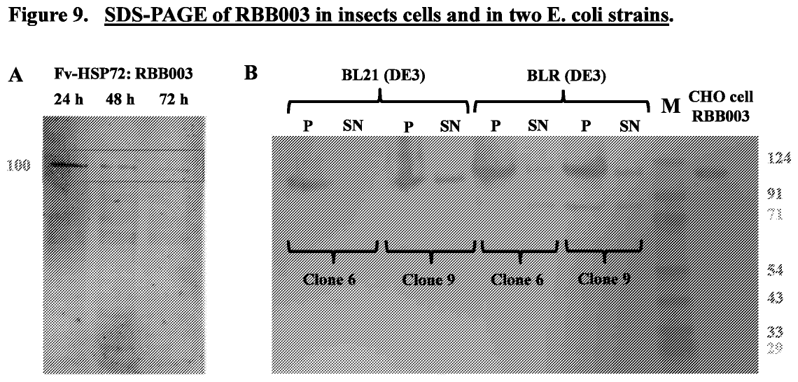

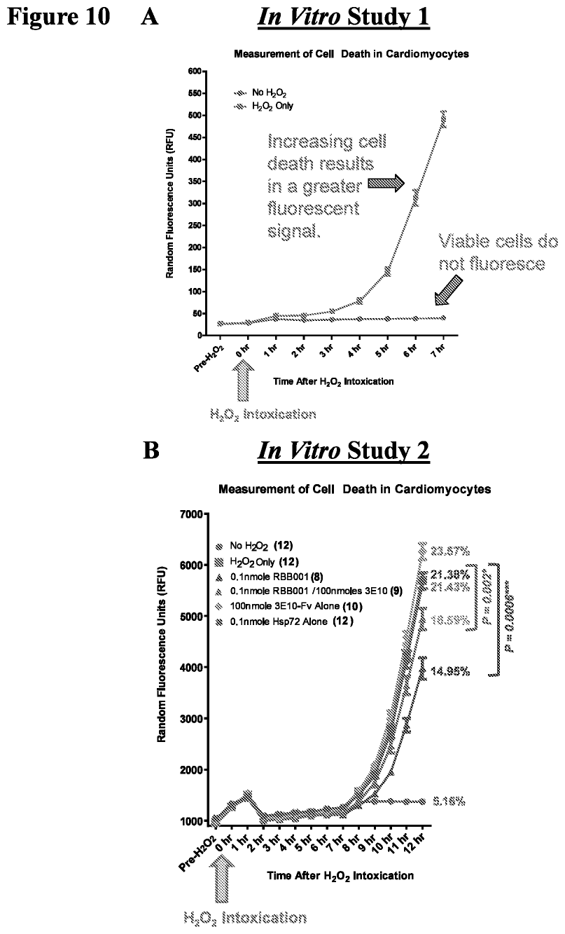

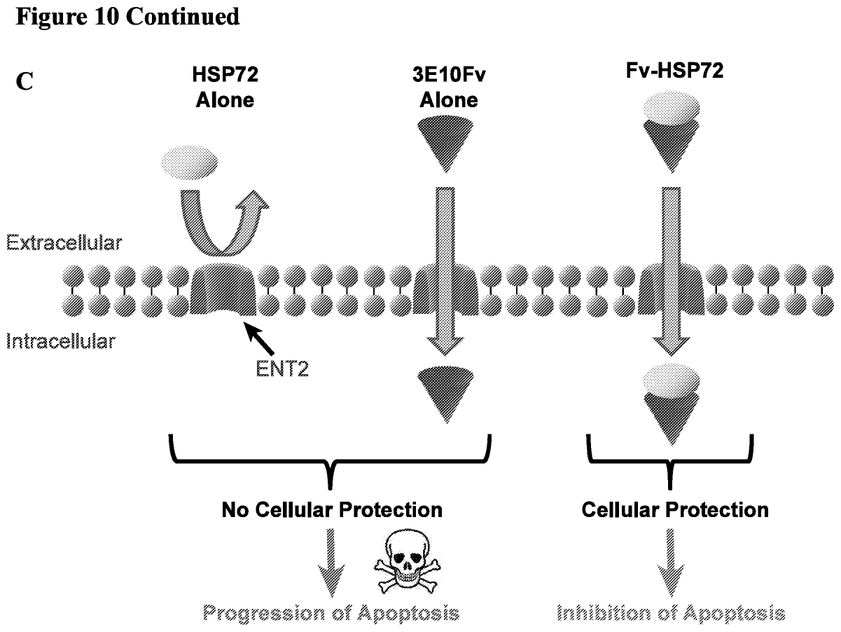

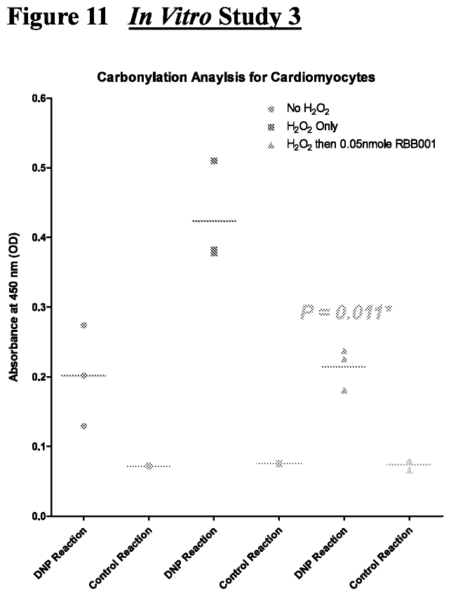

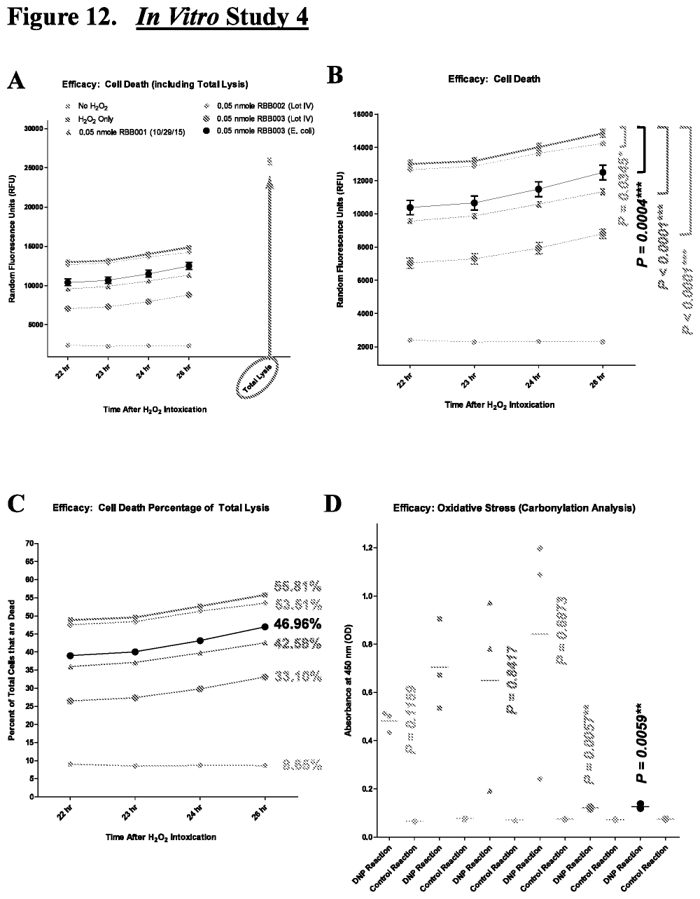

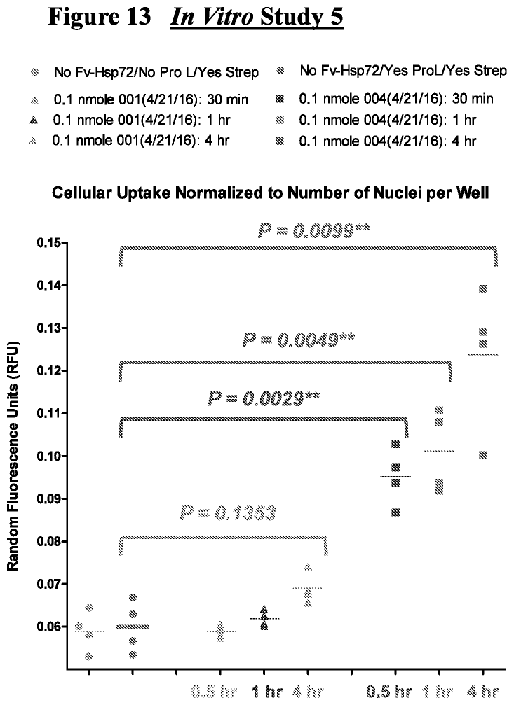

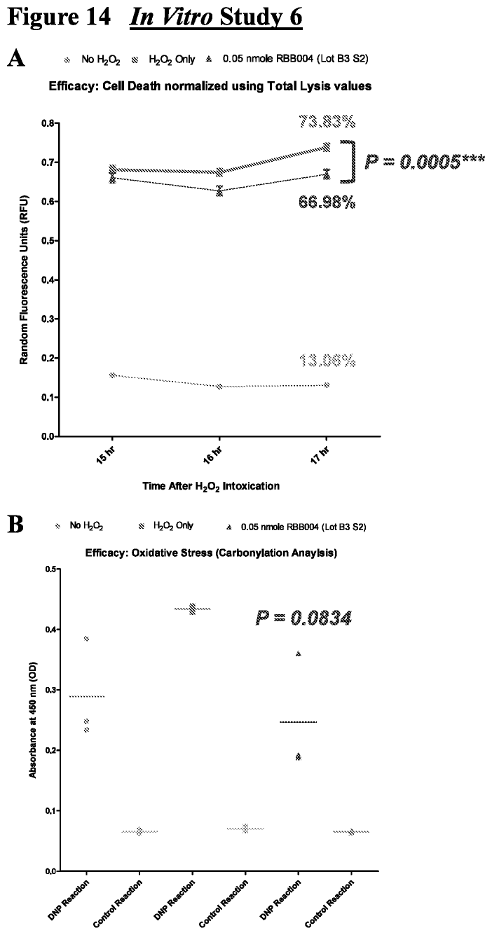

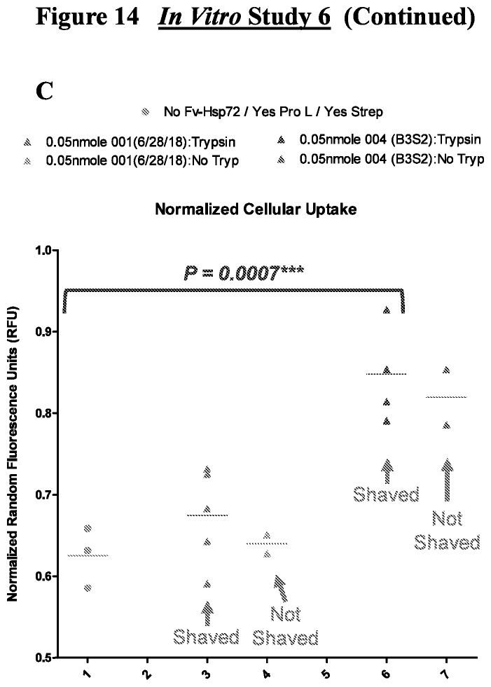

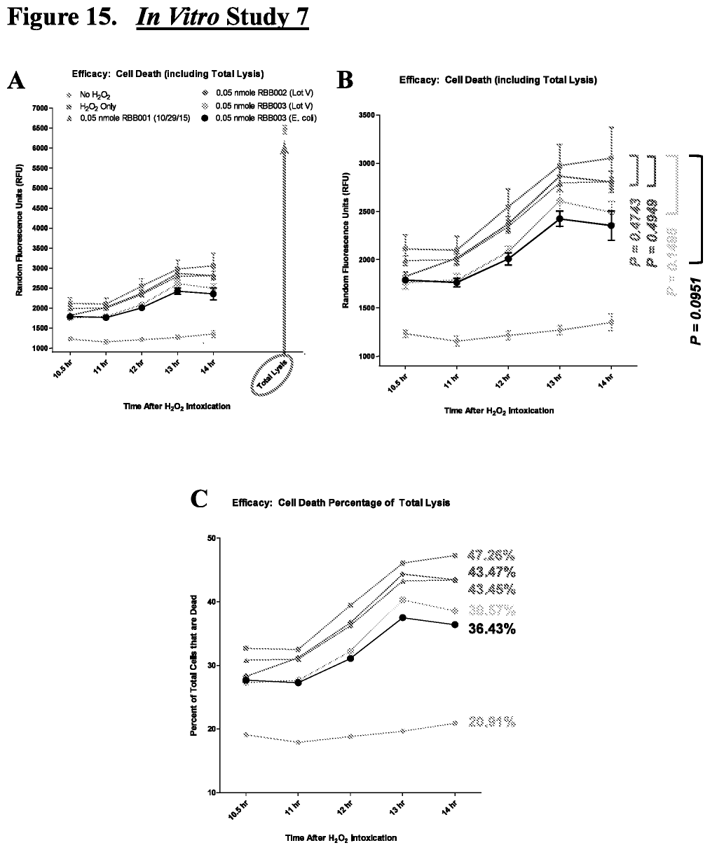

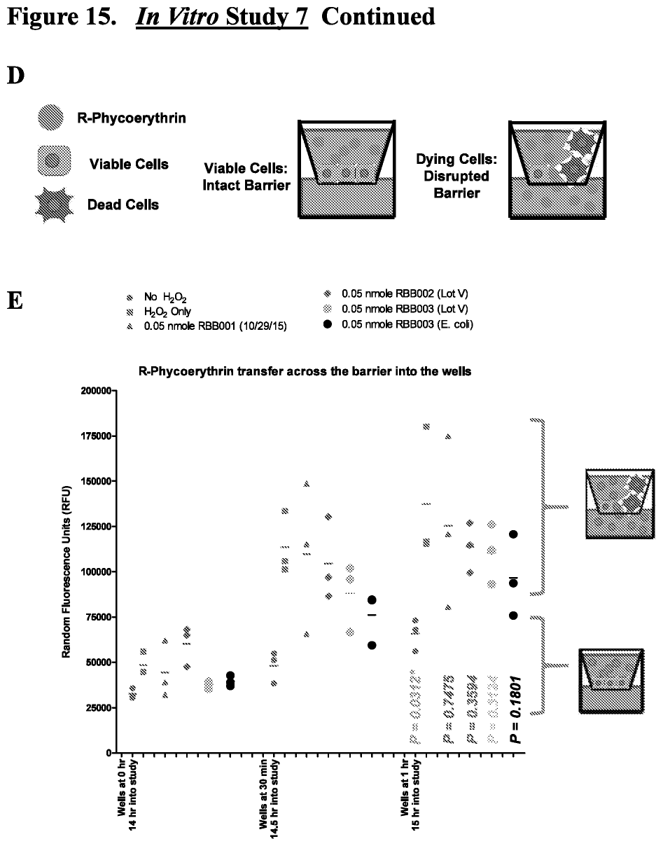

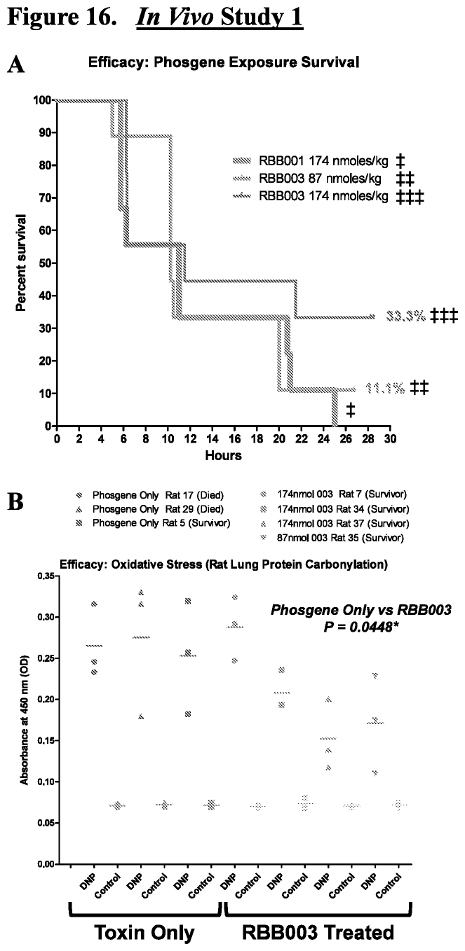

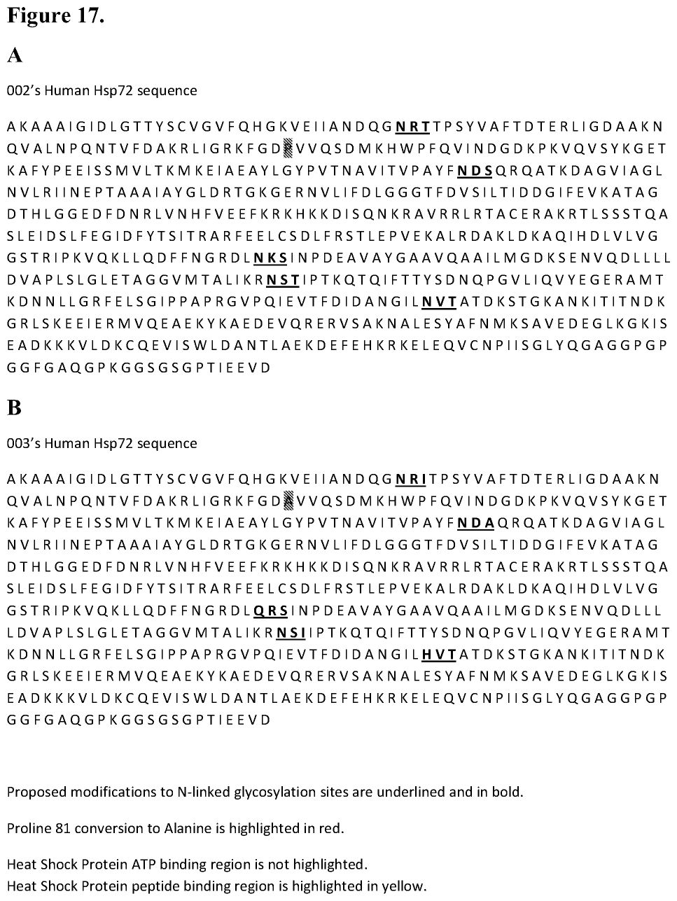

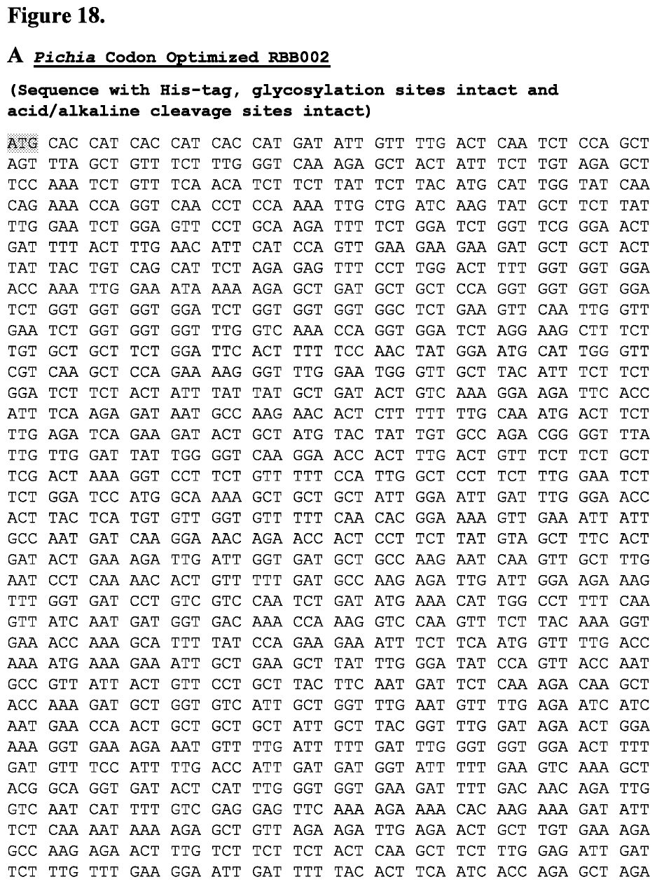

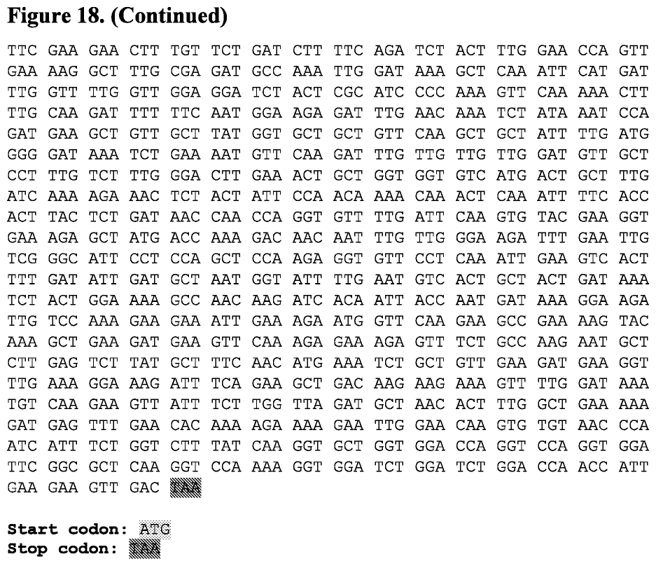

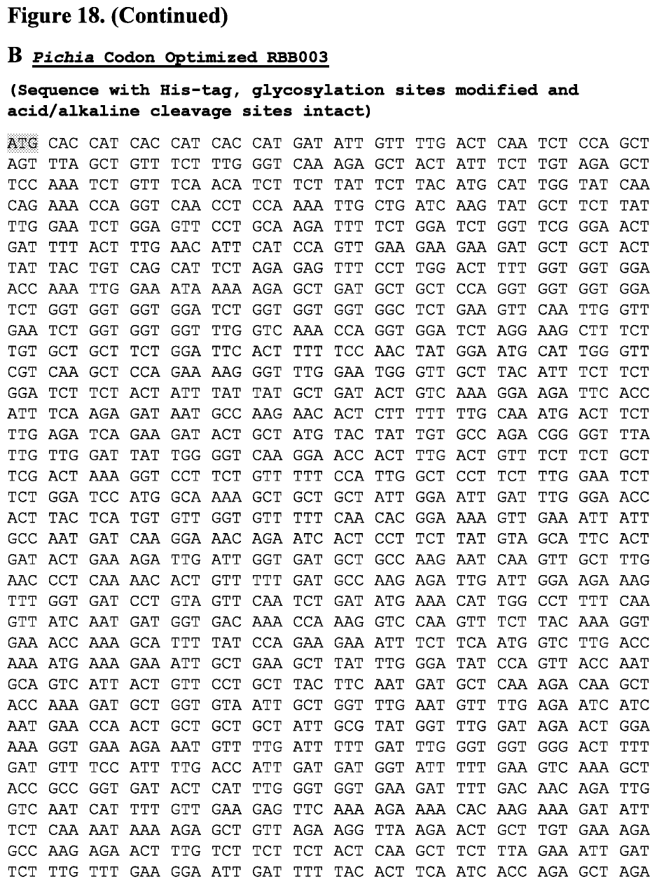

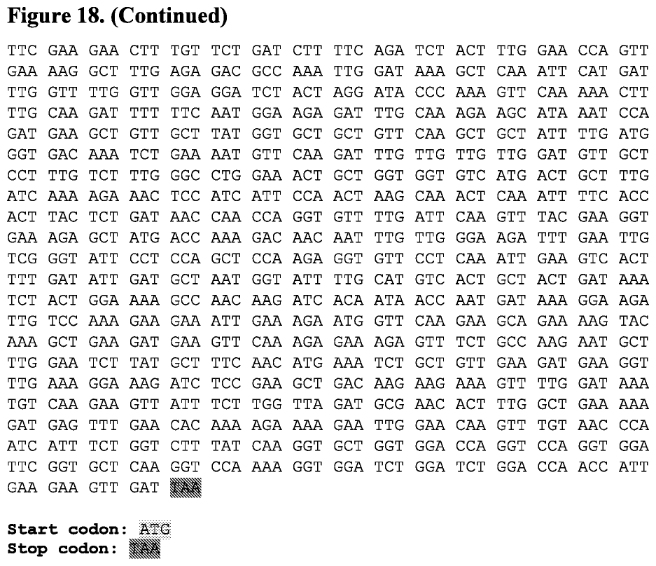

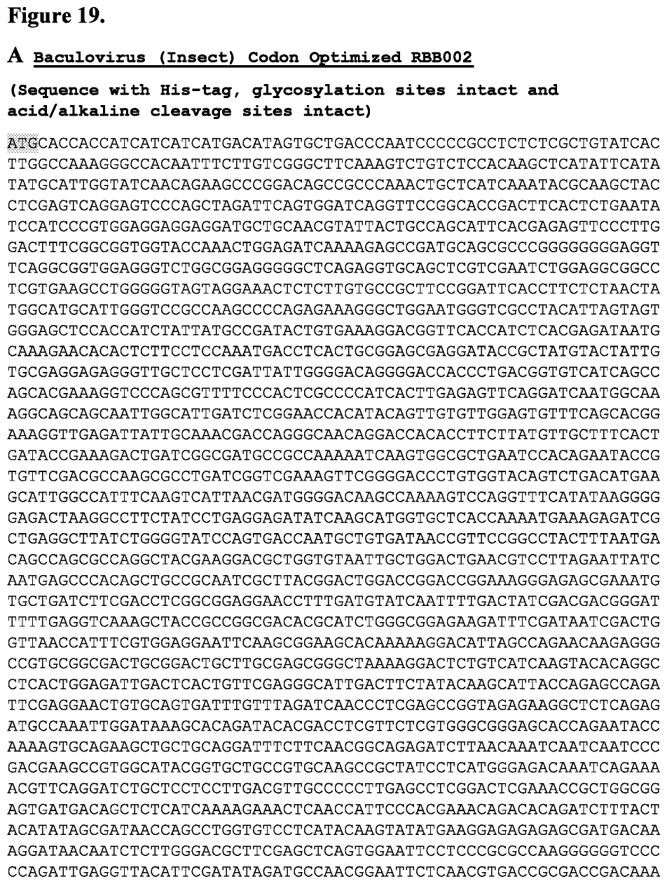

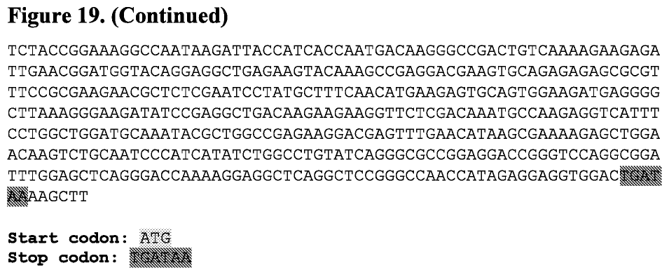

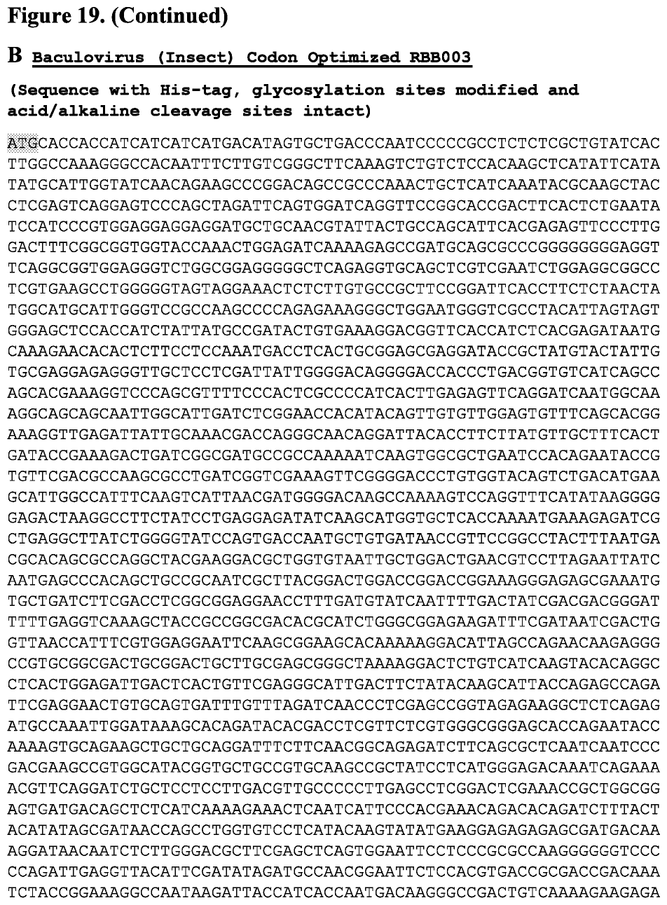

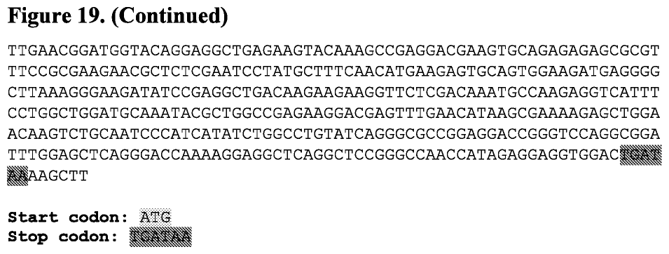

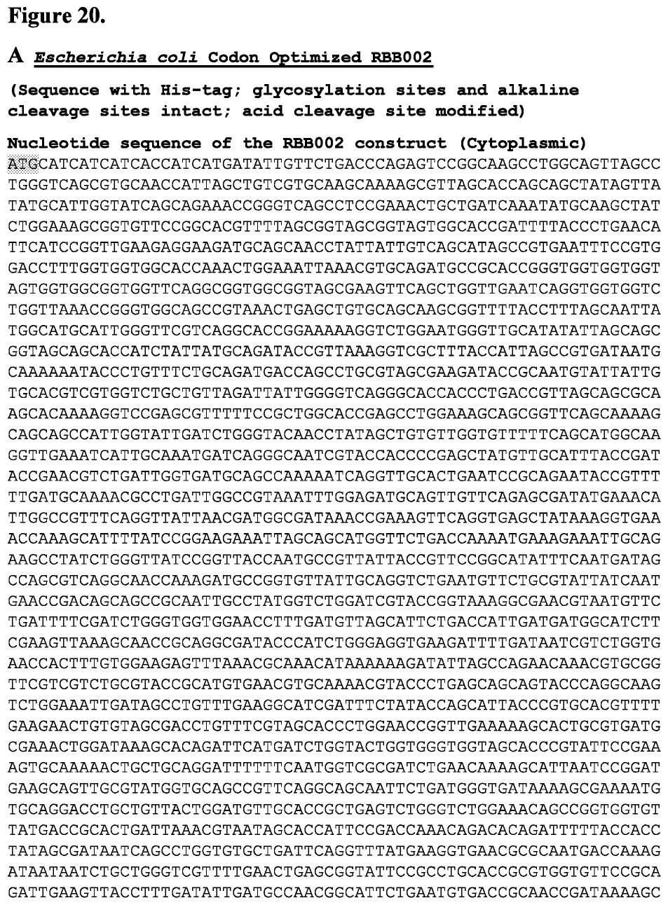

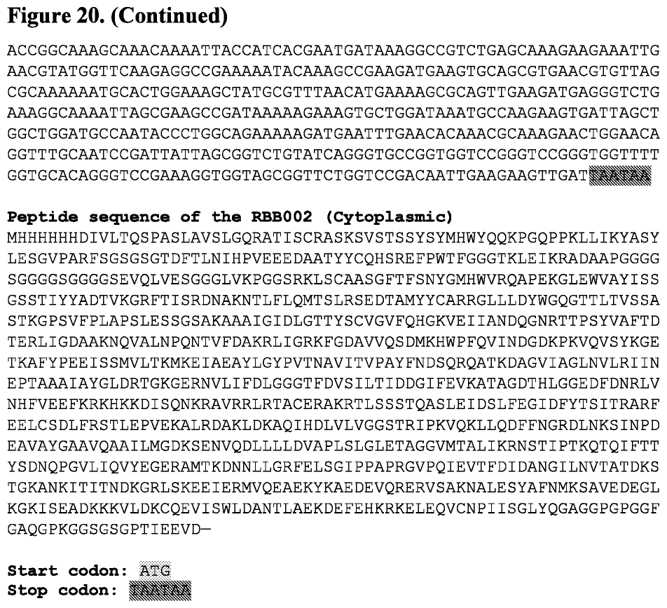

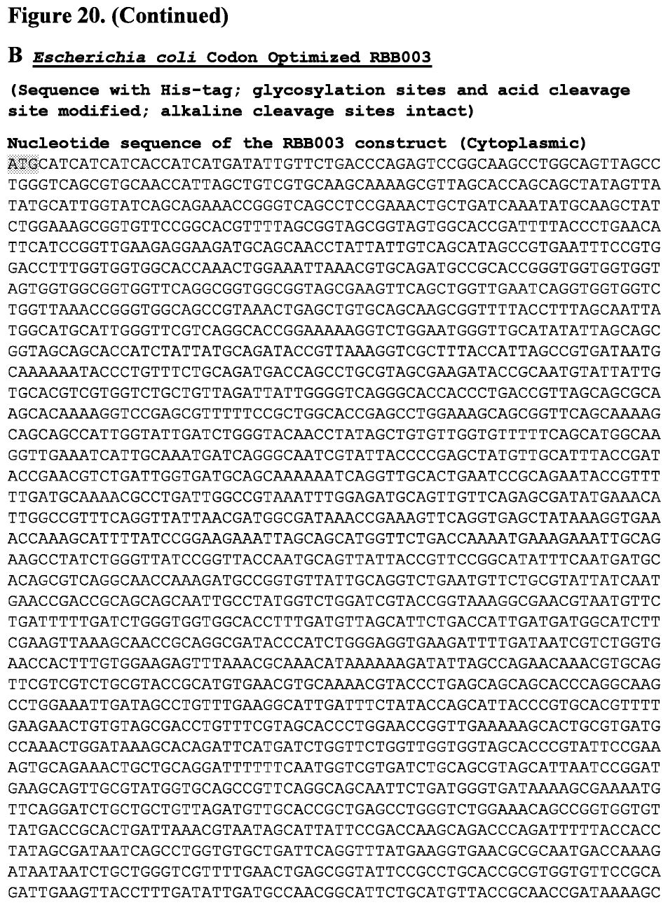

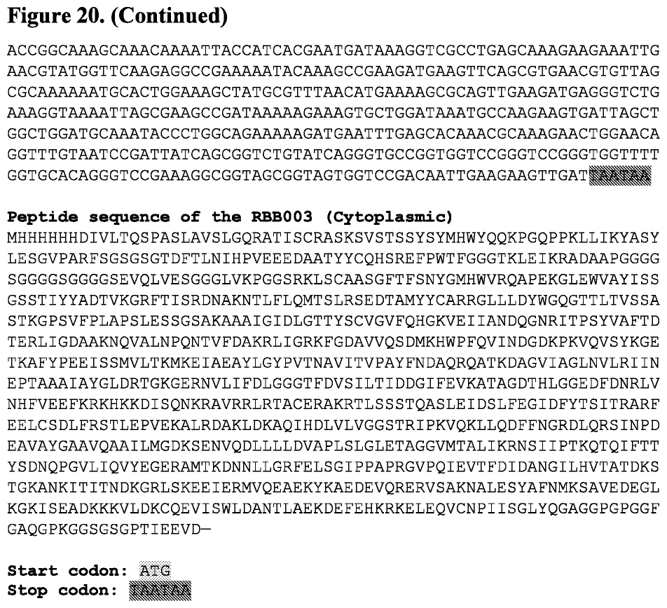

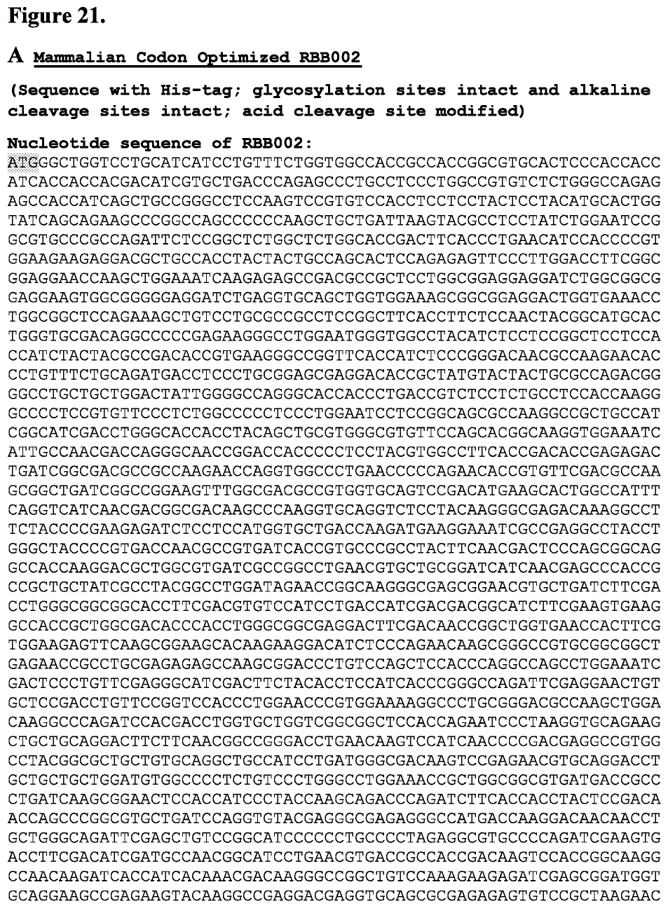

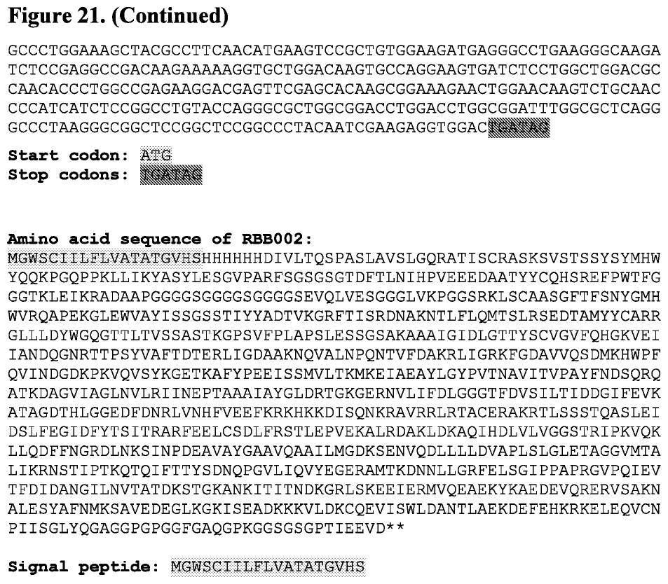

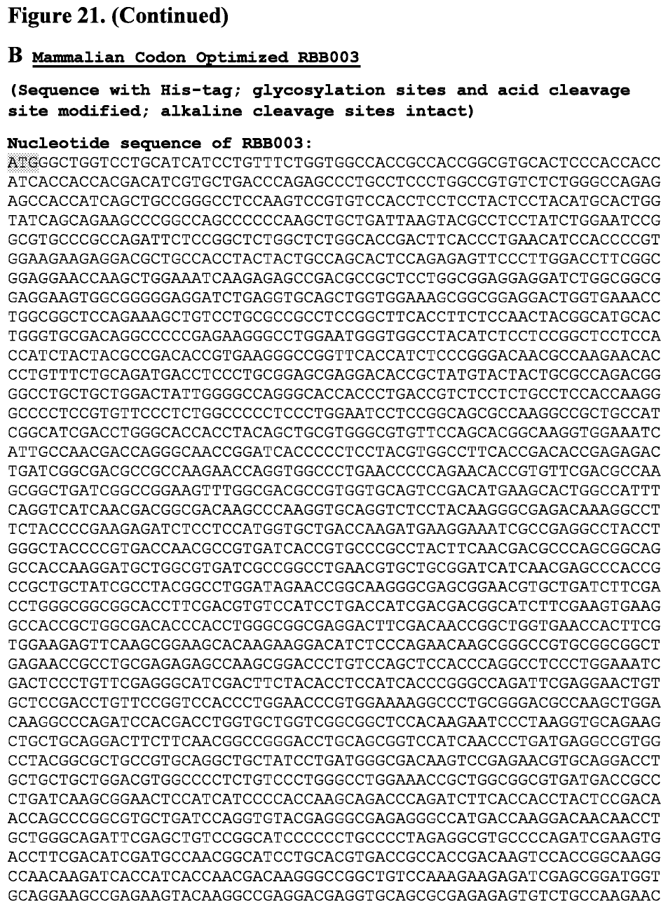

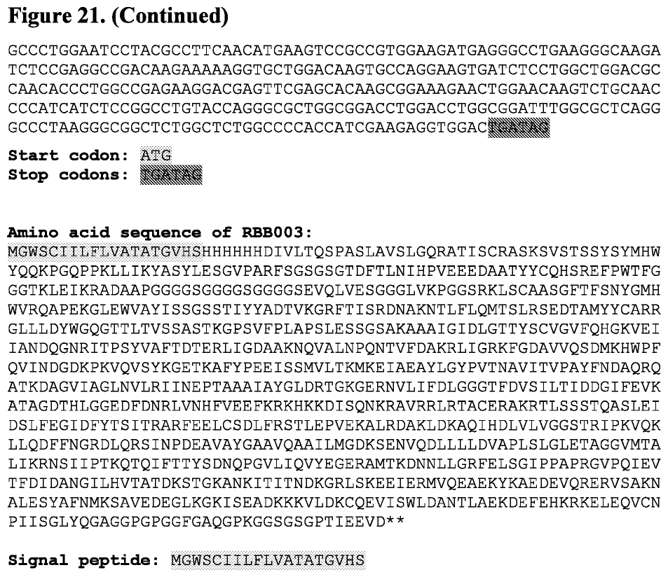

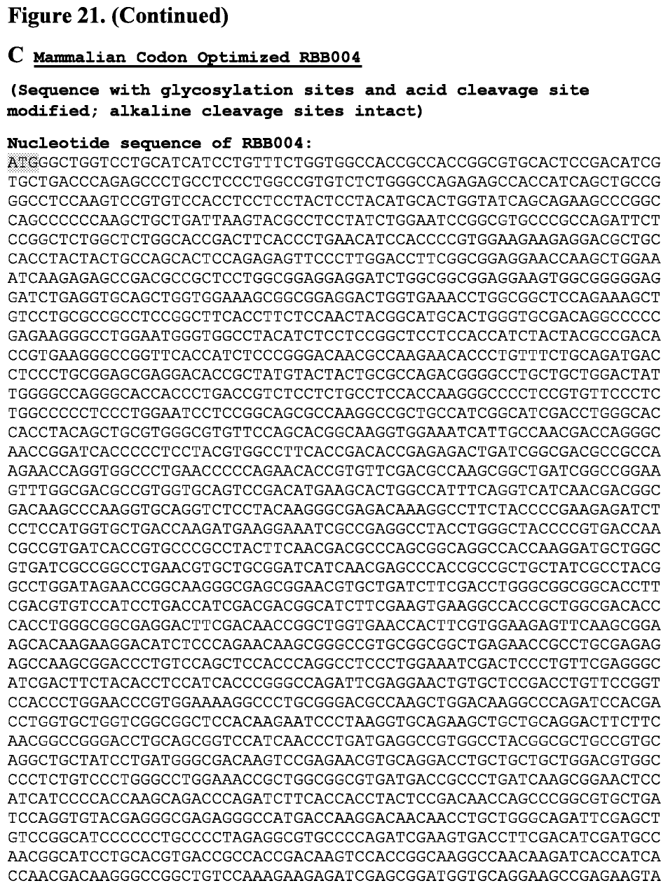

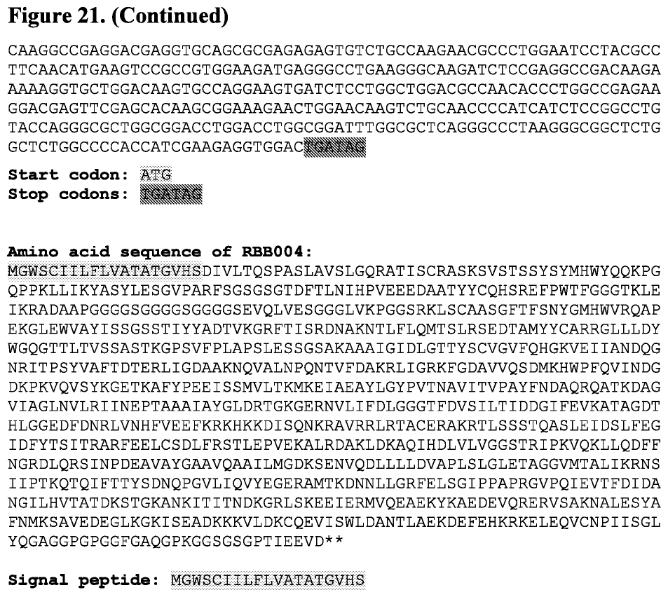

To test proof-of-concept, two second generation fusions of the 3E10 scFv with human HSP72 were created (see RBB002 and RBB003 in FIG. 7). The genes for both constructs were codon optimized for production in 1) Pichia fungal cells, 2) the Baculovirus/Sf21 and High Five insect cell systems, and 3) CHO mammalian cells. Intact RBB002 and RBB003 were produced in both Pichia (FIG. 8A) and CHO cells (FIG. 8B). The production of intact RBB003 in insect cells was also demonstrated (FIG. 9A). To further test the versatility of the RBB003 construct for production in other systems, a codon-optimized version of this Fv-HSP72 was produced for evaluation in E. coli (FIG. 9B). Enough material from the E. coli production was recovered for some comparative efficacy testing with the CHO produced RBB002 and RBB003.

Subsequently, a third generation fusion of the 3E10 scFv with human HSP72 was created, identical to RBB003, but lacking the His-tag sequence present in previous generations (see RBB004 in FIG. 7). Like RBB002 and RBB003, the acid labile D-P cleavage site of RBB004 was modified to a non-labile one by converting the proline to an alanine. Removal of the site improves product stability during low pH exposures in the purification process for these proteins. Low pH exposures may include, but are not limited to, storage buffers, product elution from affinity purification resins, or viral inactivation steps. To demonstrate the low pH stability resulting from elimination of the D-P cleavage site, RBB001, which retains the D-P cleavage site, and RBB004, which had the site converted to D-A, were both incubated in 15 mM hydrochloric acid (HCl) at 100° C. for 5, 10 and 20 minutes as described in [Marcus, F. (1985) Preferential cleavage at aspartyl-prolyl peptide bonds in dilute acid. Int. J. Peptide Protein Res. 25: 542-546]. The resulting peptide fragments in each sample were resolved by SDS-PAGE (4-20% gels) and the protein bands visualized on an ultraviolet (UV) light box using a tri-halo compound that reacts with tryptophan. The RBB004 samples presented a single band corresponding to intact protein whether boiled in water or in 15 mM HCl for each of the incubation periods. On the other hand, the RBB001 samples had multiple bands associated with the two major bands expected from a cleavage of the primary sequence at the D-P site: one ˜37.4 kD band corresponds to the primary sequence from the N-terminus to the aspartic acid-80 residue, while the other band, ˜61.5 kD, corresponds to the primary sequence from the proline-81 residue to the C-terminus. The multiple affiliated bands are due to the varying amounts of glycosylation in each protein fragment compounded with the hydrolysis of some carbohydrate sugars in the high heat, low pH conditions of the experiment.

### Example 2: In Vitro Efficacy Testing—Cell Death

One form of in vitro efficacy testing is a cell-based assay to quantitate cell death and its inhibition with HSP72 treatment. CellTox Green (Promega), a fluorescent, cell-impermeant DNA binding dye, was added to media during tissue culture incubations at 37° C., to test product potency by measuring cell death (FIG. 10A). The cytoprotective efficacy of the wild-type and modified HSP72 fusions to the 3E10 scFv were tested in cells intoxicated with hydrogen peroxide (H₂O₂) to induce oxidative stress and subsequent apoptosis, resulting in increased exposure of DNA and concomitant increases in fluorescent signal as the plasma membrane was compromised in dying cells.

The efficacy assay was developed using Rubicon's first generation Fv-HSP72 (RBB001). Wells receiving 0.1 nanomoles (nmole) of RBB001 30 minutes after the start of H₂O₂ intoxication had significantly lower cell death than untreated controls (p=0.0006). This reduction in cell death was partially compromised if the 3E10 scFv alone were added in molar excess as a competitive inhibitor prior to the RBB001 (FIG. 10B). As expected, the same molar quantity of 3E10-Fv alone (100 nmole) used in the competitive inhibition experiment did not inhibit apoptosis. Adding a molar equivalent of Hsp72 alone comparable to RBB001 (0.1 nmoles) also was not effective in reducing apoptosis when compared to cells exposed to H₂O₂ Only. Wells not intoxicated with H₂O₂ had very little cell death over the course of the experiments. To determine the maximum fluorescent signal achievable in each well and to calculate the percentage of cell death, some wells were exposed to a lysis buffer. The readings resulting from total cell death were used to normalize all of the readings as a percentage value. Whether cardiomyocyte intoxication included 12 hours of H₂O₂ exposure, overnight exposure or longer (in one study, up to 26 hours) Fv-HSP72 treatment inhibited cardiomyocyte apoptosis. The results of this study confirm the 3E10 antibody transports HSP72 into cardiomyocytes, where the heat shock protein inhibits apoptosis (FIG. 10C). Neither 3E10 nor HSP72 alone is cardioprotective.

### Example 3: In Vitro Efficacy Testing—Oxidative Stress

A secondary measure of product efficacy is to test for reductions in cellular oxidative stress brought about by the H₂O₂ intoxication (FIG. 11). Oxidative damage to cells can be quantitatively measured with protein carbonylation, a post-translational modification caused by increased reactive oxygen species (ROS) that is more stable than glutathione analysis when observing cells for a long period of time. Cells are lysed and the total protein from an SDS extraction are transferred to a tube for incubation with 2,4-dinitrophenylhydrazine (DNPH) to derivatize any carbonyl adducts formed on proteins due to oxidation. Parallel reactions are run with total protein extracts using a control solution without the DNPH. Once the reaction is completed and neutralized, the DNP derivatized proteins and their control counterparts are placed into 96-well plates and incubated with an anti-DNP primary antibody, followed by a horseradish peroxidase (HRP) conjugated secondary antibody, and an HRP colorimetric substrate (TMB; 3,3′,5,5′-tetramethylbenzidine). As seen in FIG. 11, cells exposed to H₂O₂ have greater amounts of carbonylated protein than cells not exposed to H₂O₂ or ones treated with RBB001 after H₂O₂ exposure.

### Example 4: In Vitro Study 4—Efficacy Testing RBB002 and RBB003 for Cell Death and Oxidative Stress in Cardiomyocytes

Both efficacy tests described above were used for In Vitro Study 4 to compare the cytoprotective effects of three Fv-HSP72 constructs described in FIG. 7. Human primary cardiomyocytes were seeded into 96-well plates and incubated at 5% carbon dioxide (CO₂) and 37° C. for 48 hours prior to H₂O₂ intoxication. At the start of intoxication, CellTox Green was added to the media for tracking cell death during the course of the 26 hour study. Other than the controls with No H₂O₂ and H₂O₂ Only, cells were treated 30 minutes after intoxication either with RBB001 (made in Pichia ), RBB002 (Lot IV made in CHO mammalian cells), RBB003 (Lot IV made in CHO mammalian cells), and RBB003 (made in E. coli). As seen in FIG. 12A-C, cells receiving Fv-HSP72, regardless of which construct, had significantly lower cell death than the untreated H₂O₂ Only control. However, differences were seen in the degree to which apoptosis was inhibited. Cells treated with the non-glycosylated RBB003 had the greatest inhibition (only 33.10% of the total cells lost their viability, FIG. 12C), followed by RBB001 (42.58%) and the RBB003 from E. coli (46.96%). RBB002 also showed statistically significant inhibition of apoptosis, but the 53.51% of cells that lost their viability were not much of an improvement over the 55.81% cell death seen in the 14202 Only control wells.

This lack of efficacy for RBB002 was also observed in the oxidative stress assay. Three wells from each treatment were lysed and total protein extracted with SDS as described above. DNPH labelling of carbonyl adducts to measure oxidative stress during the 26 hour intoxication, when compared to the H₂O₂ Only control, revealed significantly lower oxidation of proteins in those cells treated with RBB003 either from mammalian CHO cells or E. coli (FIG. 12D). Indeed, the oxidation levels were not only lower than the glycosylated RBB001 and RBB002, but even lower than the cells not exposed to H₂O₂ at all.

The improvement in efficacy for the mammalian CHO produced RBB003 and its non-glycosylated version of HSP72 were surprising. An Fv-HSP72 that worked as well as the RBB001 produced in fungi would have been expected, but the improvement was clearly statistically significant. The results with RBB002 were also surprising. The carbohydrate structures generated on fungal glycoproteins tend to be large branched oligo-mannose structures. Those created on mammalian glycoproteins, such as the wild-type HSP72 in RBB002, tend to be smaller oligosaccharides but with a greater heterogeneity of monosaccharide components. One would have thought a smaller set of carbohydrate structures attached to the HSP72 portion of RBB002 would have improved its efficacy over RBB001, however, this turned out not to be the case in the cardiomyocytes studied. The RBB002 construct was less efficacious in improving survival compared to cells exposed to H₂O₂ Only (53.51% vs 55.81% cell death) and it did not reduce oxidative stress levels as measured by protein carbonylation (FIG. 12D).

FIG. 12 evaluates the statistical significance of the constructs versus the H₂O₂ Only control. Since the RBB003 (Lot IV made in CHO mammalian cells) showed better efficacy than the original RBB001 Fv-HSP72 in both assays, we also conducted a Student's t-test comparing the two to gauge whether our improvements to the HSP72 translated into statistically significant results. With respect to inhibition of cell death, the difference between RBB001 and RBB003 was highly significant (p <0.0001), however, this was not the case for reduction in oxidative stress (p=0.0885). The apparent lack of significance in the second assay is due to a large variation in results obtained in the three wells treated with RBB001, not the three treated with RBB003.

### Example 5: In Vitro Study 5—Measuring Cellular Uptake

Measuring cellular uptake helps evaluate the quantity of HSP needed for efficacy on a per cell basis. Such considerations could help determine in vitro and in vivo dosages that are economical and still effective. Significantly improving the cellular uptake of the Fv-HSP72 molecule by removing the glycosylation sites was a surprising result. RBB001 and RBB004 uptake was first evaluated using a lung adenocarcinoma cell line that reached confluency rapidly and generated extracellular DNA as some of the overgrown cells died. By using this approach, cellular uptake was first evaluated independent of H₂O₂ intoxication or other traumas that can lead to cell death. FIG. 13 illustrates cellular internalization over the course of 4 hours for both Fv-HSP72s. Once it was detected using biotinylated Protein L and Streptavidin conjugated to the AlexaFluor594 (Strep-AF594), the glycosylated RBB001 did not achieve a level of uptake that was statistically significant from the background. However, the non-glycosylated RBB004 achieved statistically significant levels of uptake, and much greater than RBB001, within 30 minutes of exposure to the cells.

Cellular uptake was measured at each time point by rinsing the wells with a buffered saline solution, for example phosphate (PBS) or tris (TB S), fixing the cells with ice cold 100% ethanol, and rinsing again with buffered saline before blocking the wells with a blocking buffer. Upon completion of blocking, the cells were probed with biotinylated Protein L (1:1000 dilution), rinsed with a saline buffer containing Tween-20, probed with Streptavidin conjugated to the AlexaFluor594 (Strep-AF594) fluorochrome (1:1000 dilution), rinsed again with a saline buffer containing Tween-20, and rinsed with a saline buffer without Tween-20 and then read in a plate reader as described in FIG. 13. The cellular nuclei were then stained with a DNA stain, such as 4′,6-Diamidine-2′-phenylindole dihydrochloride (DAPI) or Hoechst 33342 (Bisbenzimide) in order to obtain a measure of cell density in each well. The wells were again read in a plate reader as described in FIG. 13. The Strep-AF594 fluorescent signal values were normalized by dividing with the DNA fluorescent signal values and then plotted as in FIG. 13. Given the strong efficacy seen with RBB001 in stroke and myocardial infarction animal models, it is expected that RBB004 will show greater efficacy due to the faster cellular internalization.

### Example 6—In Vitro Study 6: Efficacy Testing RBB004 for Cell Death, Oxidative Stress and Cellular Uptake in Cardiomyocytes

RBB004 is a third generation Fv-HSP72 that is lacking the His-tag sequence, a modification that is better suited for clinical use, hence its efficacy was evaluated in human primary cardiomyocytes. In Vitro Study 6 was carried out using the same procedures already described for In Vitro Study 4 and FIG. 14. Cells receiving RBB004 had significantly lower cell death (p=0.0005) than the untreated H₂O₂ Only control (FIG. 14A). Three wells from each treatment were lysed and total protein extracted with SDS as described in Example 3 (In Vitro Study 3). DNPH labelling of carbonyl adducts to measure oxidative stress during the 17 hour intoxication, when compared to the H₂O₂ Only control, revealed lower oxidation of proteins in those cells treated with RBB004 (FIG. 14B). While the results did not meet the statistical significance mark of p=0.05 due to one outlier in the RBB004 data set, the results were close (p=0.0834) and would likely meet the mark when a greater number of wells are surveyed.

Cellular uptake of RBB004 was compared with RBB001 in cardiomyocytes using a modified version of the procedure described for In Vitro Study 5 and FIG. 13. For this study, cardiomyocytes were intoxicated with 3 mM H₂O₂ at time T=0 hours and the cells washed with saline buffer after 4 hours. To address the possibility that some Fv-HSP72 is stuck to the extracellular surface of the cells and not internalized, the cells were briefly incubated with trypsin (5-10 seconds) in order to proteolytically digest or “shave” any RBB001 or RBB004 on the outer surface without causing the cells to detach from the plastic substrate of the multi-well plate. It was observed that detachment of cardiomyocytes occurred after 10 seconds, suggesting trypsinization was already digesting critical proteins from the cellular surface within that time frame. To stop trypsinization, cells were rinsed with a buffered saline, fixed with ice cold 100% ethanol, rinsed again with a buffered saline, before blocking the wells with a blocking buffer. Detection of Fv-HSP72 cellular uptake was performed as described for In Vitro Study 5. As was observed with the A549 cells, the RBB004 uptake was greater in cardiomyocytes than RBB001. In fact, after 4 hours, RBB001 uptake was not statistically significant (p=0.2451), whereas, RBB004 is highly significant (p=0.0007).

Based on the results from In Vitro Studies 4 and 6, removal of the glycosylation sites and an acid cleavage site from HSP72 was not detrimental to the molecule's efficacy or stability, on the contrary, these modifications improved both and increased cellular uptake as well.

### Example 7: In Vitro Study 7—Efficacy Testing RBB002 and RBB003 for Cell Death and Lung Tissue Barrier Integrity in Pulmonary Alveolar Cells

Modified HSPs for delivery as cytoprotectants are not solely intended for cardiovascular tissues, rather they should be versatile and used in, but not limited to, ocular, neurological, muscular hepatic, renal, integumentary, and pulmonary tissues as well. Hence, for In Vitro Study 7, three of the four Fv-HSP72 constructs described in FIG. 7 (RBB001, RBB002 and RBB003) were evaluated for efficacy in non-cardiac cells. Human primary alveolar cells were seeded on semi-permeable membranes (0.4 um pore size) and grown to a restrictive confluent monolayer. These confluent monolayers then had the basolateral side immersed in media and the top interfacing with the air to provide a good simulation of the lung environment in vivo and to help improve cell-cell contacts until the time of experimentation. This method allows one to assess both cell survival and barrier integrity of the confluent lung tissue.

Cells were incubated at 5% CO₂ and 37° C. prior to H₂O₂ intoxication. At the start of intoxication, CellTox Green was added to the media for tracking cell death during the course of the 14 hour study. As was the case in Study 4, controls with No H₂O₂ and H₂O₂ Only were compared with cells treated 30 minutes after intoxication either with RBB001 (made in Pichia), RBB002 (Lot V made in CHO mammalian cells), RBB003 (Lot V made in CHO mammalian cells), or RBB003 (made in E. coli). Inhibition of apoptosis was seen in all Fv-HSP72 treated cells (FIG. 15A-C). However, due to the larger than expected variation in cell death seen with the H₂O₂ Only controls, the inhibition was not significant for any of the Fv-HSP72s. It is noteworthy that alveolar cells treated with either RBB003 showed an ˜10 percentage point decrease in cell death compared to the H₂O₂ Only controls, whereas RBB001 and RBB002 only showed a 4 point decrease (FIG. 15C).

### Example 8: In Vitro Efficacy Testing—Barrier Integrity

In some embodiments, heat shock proteins in accordance with the present disclosure can be a full sequence of wild-type, naturally occurring HSP, any functional derivative or functional fragment thereof, e.g. a derivative or fragment having the same or similar activities and utilities as the corresponding wild-type protein.

Alveolar cells grown on a semi-permeable membrane simulate the lung architecture and provide yet another opportunity to test the efficacy of our cytoprotectants. In this case, the integrity of the cell-cell contacts and the barrier they create. To assess how well alveolar cells maintain their barrier integrity on 0.4 um pore size membranes, we tracked migration of a 250 kD fluorescent protein, R-Phycoerythrin (R-PE, Life Technologies) from the inserts into the underlying wells (FIG. 15D), thus, allowing for a quantifiable measure of flux across the barrier. Those inserts containing a monolayer of viable cells will retard the migration of R-PE across the semi-permeable membrane and into the well below. Cell death or shrinkage due to early apoptosis leads to disruptions in the barrier integrity and faster migration across the membrane. When compared to the H₂O₂ Only controls, migration was only significantly slowed for the No H₂O₂ cells (p=0.0312); the Fv-HSP72 constructs showing varying inhibition of R-PE migration (FIG. 15E). Although not statistically significant, the RBB003s from CHO cells and E. coli both appeared to maintain barrier integrity better than RBB001.

### Example 9: In Vivo Study 1—Efficacy Testing RBB002 and RBB003 in a Toxic Inhalation Model

In vivo testing of modified HSP72 and other modified HSPs evaluates efficacy after physiological trauma to specific tissues. One such trauma is the inhalation of toxins, which can result in cell death and oxidative stress. In a series of studies to extend work that was being conducted with alveolar cells in vitro, the RBB002 and RBB003 manufactured in CHO mammalian cells and the RBB001 made in Pichia were tested in a toxic inhalation model; one where damage to lung cells rapidly leads to death. Efficacy for each of the Fv-HSP72s was evaluated using survival and oxidative stress.

The toxic inhalation model used challenges rats to phosgene vapor, a deadly, but industrially important, chemical whose release into the atmosphere and acute inhalation can result in airway exfoliation, terminal airway edema and hemorrhaging. Treatment for phosgene exposure in the workplace and on the battlefield is an unmet medical need. Rats were challenged with phosgene for 10 minutes at lethal doses then administered, 30 minutes later, either RBB001, RBB002 or RBB003 through an intravenous (IV) tail vein injection. Control rats received no treatment. All work was done in special containment facilities to prevent accidental human exposure. Survival was monitored for 25 hours post-exposure. Given the difficulties of consistently working with phosgene's very steep toxicity curve, even when carefully monitoring delivery of the toxin by vapor to the rats, limited numbers of animals had to be handled and studied at a time. Hence, a single comprehensive study of all constructs could not be conducted at one time if a statistically meaningful numbers of rats were to be used.

In one study, administration of a 174 nmoles/kg RBB002 dose did not result in improved survival over rats solely receiving phosgene without any Fv-HSP72 treatment (n=9 rats/Phosgene Only group and n =8 rats / RBB002 treatment group; data not shown). In a similar study, the modified HSP72 found in RBB003 appeared more efficacious than the wild-type HSP72 in RBB001. An 87 nmole/kg dosage of RBB003 had only 1 out of 9 rats surviving after 24 hours, while double that dose had 3 out of 9 surviving (FIG. 16A). None of the rats receiving 174 nmoles/kg of freshly prepared RBB001 survived. All survivors in both studies lacked the red mottled appearance indicative of hemorrhaging, and had lung/body weight ratios<0.02 suggesting a non-lethal amount of airway edema. Furthermore, total protein extracted from lung tissues of rats treated with RBB003 had statistically significant reductions in oxidative stress, as measured by carbonylation, than tissue from rats that were left untreated (p=0.0448; FIG. 16B).

The results from this pulmonary study of toxic inhalants, combined with the in vitro results, allow some conclusions to be drawn. The in vitro results obtained with multiple lots of RBB002 in cardiomyocytes, alveolar cells and alveolar carcinoma cells (data not shown), portended the lack of efficacy seen with this Fv-HSP72 in vivo. The disruption in efficacy caused by a shift in glycosylation moieties from the large oligo-mannose structures seen in Pichia for RBB001 to the more heterogenous oligosaccharides seen in mammals for RBB002 was surprising.

More surprising was the superior efficacy of the modified HSP72 sequence in RBB003 when the glycosylation sites were eliminated with a new primary sequence. Both in vitro and in vivo, RBB003 proved more efficacious than the Fv-HSP72 constructs containing the wild-type human HSP72 sequence. In the case of the pulmonary study, the 87 nmoles/kg dose of RBB003 was likely not effective given the one surviving rat; however, a comparison of both RBB003 and RBB001 at the 174 nmoles/kg dose showed a clear difference in efficacy with three survivors versus none, respectively. A 174 nmoles/kg dose of RBB003 may not be the optimal dose, but some rats clearly benefited. It is also important to note that RBB003 was able to inhibit oxidative stress in surviving rats regardless of which dosage used as determined in a comparison with control rats who received Phosgene Only. The fact that one Phosgene Only survivor and one RBB003 survivor both had similar carbonylation levels leaves open the question of the role of oxidative stress when toxins are inhaled (FIG. 16B).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A modified heat shock protein or fragment thereof in which one or more of N-glycosylation sites is modified such that N-glycosylation at the modified site is substantially reduced as compared to N-glycosylation at the corresponding unmodified site.

2. The modified heat shock protein or fragment thereof according to claim 1, wherein said one or more of N-glycosylation sites comprises an amino acid sequence of N-X-S/T/C, wherein X is any amino acid except proline.

3. The modified heat shock protein or fragment thereof according to any one of claims 1-2, wherein one or more of acid-labile cleavage sites and/or one or more of alkaline-labile cleavage sites is modified such that cleavage at the modified cleavage site at or under pH 4, 5, 6, or 7; or at or above pH 7, 8, 9, 10, 11 or 12 is substantially reduced as compared to cleavage at the corresponding unmodified cleavage site.

4. The modified heat shock protein or fragment thereof according to claim 3, wherein said one or more of acid-labile cleavage site comprises an amino acid sequence of D-P.

5. The modified heat shock protein or fragment thereof according to claim 3, wherein said one or more of alkaline-labile cleavage site comprises an amino acid sequence of N-P, N-L, N-S or N-T.

6. The modified heat shock protein or fragment thereof according to any one of claims 1-5, wherein said modified heat shock protein is selected from the group consisting of modified chaperonins, modified HSP27, modified HSP40, modified HSP60, modified HSP70, modified HSP90, modified HSP105/110 and modified small heat shock proteins.

7. The modified heat shock protein or fragment thereof according to any one of claims 1-6, wherein all N-glycosylation sites are modified such that there is no N-glycosylation in said modified heat shock protein or fragment thereof.

8. The modified heat shock protein or fragment thereof according to any one of claims 1-7, wherein N-glycosylation in said modified heat shock protein or fragment thereof is reduced by about 10%, about 20%, about 30%, about 40%, about 50%, about 60%, about 70%, about 80%, about 90%, about 95%, about 99%, or about 100% as compared to N-glycosylation of the corresponding unmodified heat shock protein or fragment thereof

9. The modified heat shock protein or fragment thereof according to any one of claims 1-8, wherein about 10%, about 20%, about 30%, about 40%, about 50%, about 60%, about 70%, about 80%, about 90%, about 95%, about 99%, or about 100% of the total number of the N-glycosylation sites is modified in said heat shock protein or fragment thereof.

10. The modified heat shock protein or fragment thereof according to any one of claims 3-9, wherein all acid-labile cleavage sites are modified such that there is no cleavage of said modified heat shock protein or fragment thereof at or under pH 4, 5, 6, or 7.

11. The modified heat shock protein or fragment thereof according to any one of claims 3-10, wherein all alkaline-labile cleavage sites are modified such that there is no cleavage of said modified heat shock protein or fragment thereof at or above pH 7, 8, 9, 10, 11, or 12.

12. The modified heat shock protein or fragment thereof according to any one of claims 3-11, wherein all acid-labile and alkaline-labile cleavage sites are modified such that there is no cleavage of said modified heat shock protein or fragment thereof at or under pH 4, 5, 6, or 7; or at or above pH 7, 8, 9, 10, 11 or 12.

13. The modified heat shock protein or fragment thereof according to according to any one of claims 1-12, wherein cleavage at or under pH 4, 5, 6, or 7; or at or above pH 7, 8, 9, 10, 11 or 12 in said modified heat shock protein or fragment thereof is reduced by about 10%, about 20%, about 30%, about 40%, about 50%, about 60%, about 70%, about 80%, about 90%, about 95%, about 99%, or about 100% as compared to cleavage in the corresponding unmodified heat shock protein or fragment thereof.

14. The modified heat shock protein or fragment thereof according to any one of claims 1-13, wherein about 10%, about 20%, about 30%, about 40%, about 50%, about 60%, about 70%, about 80%, about 90%, about 95%, about 99%, or about 100% of the total number of the acid-labile and/or alkaline-labile sites is modified in said heat shock protein or fragment thereof.

15. The modified heat shock protein or fragment thereof according to any one of claims 1-14, wherein:
about 10%, about 20%, about 30%, about 40%, about 50%, about 60%, about 70%, about 80%, about 90%, about 95%, about 99%, or about 100% of the total number of the N-glycosylation sites is modified; and
about 10%, about 20%, about 30%, about 40%, about 50%, about 60%, about 70%, about 80%, about 90%, about 95%, about 99%, or about 100% of the total number of the acid-labile and/or alkaline-labile sites is modified in said heat shock protein or fragment thereof.

16. The modified heat shock protein or fragment thereof according to any one of claims 3-15, wherein all N-glycosylation sites are modified but not all acid-labile cleavage sites and not all alkaline-labile cleavage sites are modified in said heat shock protein or fragment thereof.

17. The modified heat shock protein or fragment thereof according to any one of claims 3-15, wherein all N-glycosylation sites and all acid-labile cleavage sites are modified but not all alkaline-labile cleavage sites are modified in said heat shock protein or fragment thereof.

18. The modified heat shock protein or fragment thereof according to any one of claims 3-15, wherein all N-glycosylation sites and all alkaline-labile cleavage sites are modified but not all acid-labile cleavage sites are modified in said heat shock protein or fragment thereof.

19. The modified heat shock protein or fragment thereof according to any one of claims 3-15, wherein all N-glycosylation sites and all acid-labile cleavage sites and all alkaline-labile cleavage sites are modified in said heat shock protein or fragment thereof.

20. A pharmaceutical composition comprising the modified heat shock protein or fragment thereof according to any one of claims 1-19 and a pharmaceutically acceptable excipient.

21. The pharmaceutical composition according to claim 20, wherein the pharmaceutical composition formulated into a liposome.

22. A conjugate compound comprising the modified heat shock protein or fragment thereof according to any one of claims 1-19 and an additional compound.

23. The conjugate compound according to claim 22, wherein the additional compound comprises a peptide that is capable of transporting said modified heat shock protein or fragment thereof across a lipid bilayer.

24. The conjugate compound according to any of claims 22-23, wherein the additional compound comprises an antibody or fragment thereof

25. The conjugate compound according to claim 24, wherein said antibody or fragment thereof is capable of binding to a molecule present inside or outside a cell.

26. The conjugate compound according to claim 25, wherein said molecule present inside a cell is DNA, RNA or protein.

27. The conjugate compound according to claim 25, wherein said the molecule is located outside a cell.

28. The conjugate compound according to claim 24, wherein the antibody is 3E10.

29. The conjugate compound according to claim 27, wherein said 3E10 antibody comprises an amino acid sequence selected from the group consisting of a murine sequence, a humanized sequence, a fully human analog, or any modified form thereof

30. The conjugate compound according to any of claims 22-29, wherein the additional compound is selected from the group consisting of histones and fragments thereof, high mobility group proteins (HMGs) and fragments thereof, transcription factors and fragments thereof and poly-cation sequences comprising a plurality of lysine and/or arginine and fragments thereof.

31. The conjugate compound according to any of claims 22-30, wherein the additional compound is selected from the group consisting of phospholipid binding proteins and fragments thereof.

32. The conjugate compound according to claim 31, wherein said phospholipid binding proteins and fragments thereof are selected from the group consisting of annexins, lactadherin, sphingomyelin, apolipoprotein-H (β-2-glycoprotein-1), T-cell immunoglobulin mucin domain (TIM) receptors, galectins, and fragments of any thereof.

33. The conjugate compound according to any of claims 22-32, wherein the additional compound is selected from the group consisting of cell-penetrating peptides and fragments thereof.

34. The conjugate compound according to claim 33, wherein said cell-penetrating peptides and fragments thereof are selected from the group consisting of TAT proteins of HIV and Antennapedia proteins from insects and fragments thereof.

35. The conjugate compound according to any of claims 22-34, wherein the additional compound is selected from the group consisting of peptides and fragments thereof that bind to ions transported across the membrane.

36. A pharmaceutical composition comprising the conjugate compound according to any one of claims 22 -35 and a pharmaceutically acceptable excipient.

37. The pharmaceutical composition according to claim 36, wherein the pharmaceutical composition formulated into a liposome.

38. A method of making the modified heat shock protein or fragment thereof of any one of claims 1-19, said method comprising:
modifying at least one or more of N-glycosylation sites in the heat shock protein or fragment thereof such that N-glycosylation at the modified site is substantially reduced as compared to N-glycosylation at the corresponding unmodified site.

39. The method according to claim 38 further comprising:
modifying at least one or more of acid-labile cleavage sites in the heat shock protein or fragment thereof such that cleavage at the modified site at or under pH 4, 5, 6, or 7 is substantially reduced as compared to cleavage at the corresponding unmodified cleavage site.

40. The method according to claim 38 further comprising:
modifying at least one or more of alkaline-labile cleavage sites in the heat shock protein or fragment thereof such that cleavage at the modified cleavage site at or above pH 7, 8, 9, 10, 11 or 12 is substantially reduced as compared to cleavage at the corresponding unmodified cleavage site.

41. The method according to claim 38 further comprising:
modifying at least one or more of acid-labile cleavage sites and at least one or more of alkaline-labile cleavage sites in the heat shock protein or fragment thereof such that cleavage at the modified cleavage site at or under pH 4, 5, 6, or 7; or at or above pH 7, 8, 9, 10, 11 or 12 is substantially reduced as compared to cleavage at the corresponding unmodified cleavage site.

42. A method of treating a disease or condition in a subject comprising:
administering a pharmaceutically effective amount of the modified heat shock protein or fragment thereof of any one of claims 1-19 to the subject in need of the treatment.

43. The method of claim according to claim 42, wherein said disease or condition is selected from the group consisting of ocular, neurological, muscular, hepatic, renal, integumentary, cardiovascular, and pulmonary conditions and traumas.

43. A method of treating cells ex vivo or in vitro comprising adding an effective amount of the modified heat shock protein or fragment thereof of any one of claims 1-19 to a culture media comprising the cells to be treated.

44. The method of claim 43, wherein the method maintains the viability of the treated cells during their culture and propagation.


SEQUENCE LISTING (59 sequences)

SEQ ID NO: 1 - protein, 641 aa, Homo sapiens
  Human HSPA1A HSP72 (P0DMV8), Figure 1
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 2 - protein, 641 aa, Mus musculus
  Mouse HSPA1A HSP72(Q61696), Figure 1
MAKNTAIGIDLGTTYSCVGVFQHGKVEIIANDQGNRTTPSYVAFTDTERLIGDAAKNQVALNPQNTVFDAKRLIGRKFGDAVVQSDMKHWPFQVVNDGDKPKVQVNYKGESRSFFPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVSHFVEEFKRKHKKDISQNKRAVRRLRTACERAKRTLSSSTQASLEIDSLFEGIDFYTSITRARFEELCSDLFRGTLEPVEKALRDAKMDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTRDNNLLGRFELSGIPPAPRGVPQIEVTFDIDANGILNVTATDKSTGKANKITITNDKGRLSKEEIERMVQEAERYKAEDEVQRDRVAAKNALESYAFNMKSAVEDEGLKGKLSEADKKKVLDKCQEVISWLDSNTLADKEEFVHKREELERVCSPIISGLYQGAGAPGAGGFGAQAPKGASGSGPTIEEVD

SEQ ID NO: 3 - protein, 679 aa, Oryctolagus cuniculus
  Rabbit HSPA9 GRP75(G1SRF7), Figure 1
MISASRAAAARLVGAAASRGPAAARHQDGWNGFSHEAFRIVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHAAKNAVITVPAYFNDSQRQATKDAGQISGLNVLRVINEPTAAALAYGLDKSEDKIIAVYDLGGGTFDISILEIQKGVFEVKSTNGDTFLGGEDFDQALLRHIVKEFKRETGVDLTKDNMALQRVREAAEKAKCELSSSVQTDINLPYLTMDASGPKHLNMKLTRAQFEGIVADLIKRTMAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 4 - protein, 655 aa, Oryctolagus cuniculus
  Rabbit HSPA5 BiP(G1U7L4), Figure 1
KMKLSLVAAVLLLLGAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKYSDFRMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 5 - protein, 649 aa, Neobenedenia sp.
  Tapeworm (F8J498), Figure 1
MSKAPAIGIDLGTTYSCVGVFQHGKVEIIANDQGNRTTPSYVAFSDTERLIGDAAKNQVAMNPTNTVFDAKRLIGRKYDDPSVSSDMKHWPFNVVSEGGKPKIKVEFRGEQKTFFPEEISSMVLLKMKETAEAYLGKTVSDAVITVPAYFNDSQRQATKDAGAISGMNVLRIINEPTAAAIAYGLDKKVGTERNVLIFDLCGGTFDVSILTIEEGIFEVKSTAGDTHLGGEDFDNRLVDFFIAEFKRKNKKDISDNKRAVRRLRTSCERAKRTLSSSTQASLEIDSLYDGIDFYTSITRARFEEMCSDLFRGTLEPVEKSLRDAKCSKGDIHDIVLVGGSTRIPKIQKLLQDFFSGKELNKSINPDEAVAYGAAVQAAILSGDKSEAVQDLLLLDVAPLSLGLETAGGVMTALIKRNTTIPTKQAQTFTTYSDNQPGVLIQVYEGERAMTKDNNLLGKFELSGIPPAPRGVPQIEVTFDIDANGILNVSACDKSTGKSNKITITNDKGRLSKEEIERMVQDAEKYKADDEAVKDRVAAKNALESYAYSMKSTVEDDKVKDKISEEERKSIIDKCSEVITWLDSNQTAEKDEFEHQQKELEKVCTPVVTKMYQGAGGMPGGMPGGMPGGFPGAGGEAGAGGKGPTIEEVD

SEQ ID NO: 6 - protein, 648 aa, Dugesia sp.
  Dugesi Tapeworm (B0LVF7), Figure 1
MSKGPAVGIDLGTTYSCVGVFQHGKVEIIANDQGNRTTPSYVAFTDTERLIGDAAKNQVAMNPSNTVFDAKRLIGRRFDDATVQSDMKHWSFDVVCEGGRPKIQVNYKGELKKFFPEEISSMVLLKMKETAEGYLGRPVTDAVVTVPAYFNDSQRQATKDAGAISGLNVLRIINEPTAAAIAYGLDKKVGIERNVLIFDLGGGTFDVSILTIEDGIFEVKSTAGDTHLGGEDFDNRMVNHFVQEFKRKHKKDISDNKRAVRRLRTACERARRTLSSSAQANIEIDSLYEGIDFYTSMTRARFEELNADLFRSTLEPVEKSLRDAKMDKKDIHEIVLVGGSTRIPKIQKLLQDLFHGKELNKSINPDEAVAYGAAVQAAILSGDKSEAVQDLLLLDVAPLSLGIETAGGVMTALIKRNTTIPTKQTQTFTTYSDNQPGVLIQVYEGERAMTRDNNLLGKFELSGIPPAPRGVPQVEVTFDIDANGILNVSAIEKSTGKENKITISNDKGRLSKDDIERMVNEAEKYKQQDELQKDRVAAKNALESCSYQMKTTMEDEKLKDKLSESDRKTIIDKCSEVIGWLDHNQTAEKEEYEHHQKELEKVCNPIMTKFYQASGGMPGGFPGAAGGGPGAPGAHGTAGQGPTIEEVD

SEQ ID NO: 7 - protein, 306 aa, Echinococcus multilocularis
  Fox Tapeworm (A0A087VWV9), Figure 1
MLSAAASTKIAVESLFDEVDFCKTLARAEFEQLCMDLFSQTMDKVETTLSDAKLRKADIHEILLVGGSTRIPKVQSMLQDFFNGRDLQRSINPDEAVAYGAAVLAAKLSGNMSKLMENLMLYEVTPLSLGWKDCYGAMVTVIKRNTRIPTKRTCSSQTASDNQTSVRTSIFEGERAMAKDNNFLGEFLVQGFPAKPRGEINITITKYKGHLSEEEAKLMLAEAEKFKQEDEKERSRVAAMNALLDCIYSTKRKLEKVEVKQKMSEKNRKMLLAKCEESIKWADREKQATKEDYERKRKQFESECSL

SEQ ID NO: 8 - protein, 606 aa, Echinococcus granulosus
  Dog Tapeworm (A0A068X2F4), Figure 1
MSTSSAIGIDLGTTFSCVGVFQNNRVEIIANDQGNRTTPSCVAFTDKGRLTGEAAINQAALNPANTVCGVKRLIGCRFDDEAVQSDMKYWPFKVTNSEGKPKIEVQHCGETKHFVAEQISAMVLSKMKDTAETYLGEKVTDAVITVPAYFNNNQRNATIDAAKIAGLNVLRLINEPTAAAIAYGMDRKSERQHNVLIFDWGGGTFDVSILSINNREFDVKAVGGDTHLGGEDINSRLVDHCVEMIKQAHAGKDLTTNKKAIHRLRKACEKAKRMLSSAESTTIVVESLFYEIDFCKTLTRAEFEQLCMDLFSKTMDAVKMALSDAKMDKADIHEILLVGGSTRIPKVQSMLQDFFNGRDLQRSINPDEAVAYGATLLAANLTGGASNSMQDLMLLEVTPLSLGWENAYGKMHTVIKRNTRIPTKQACKSRTHGDNQTCVRTKIFEGERVMAKDNHFLGEFLLEGFPAAPRGETQFVSTFEIDESGMLHVSKVERSTKKQNGITIFNYKGRLSEEEIEQMLAEAEKFKQEDDKERSRVAALNALVDCIYSIKRKLEKEDIKQKISEKYRQTLLAKCEEMIKWTDTKKQATKEEYEEMRKHFEGACNL

SEQ ID NO: 9 - protein, 645 aa, Zea mays
  Maize (P11143), Figure 1
  misc_feature (614)..(614): Xaa can be any naturally occurring amino acid
MAKGEGPAIGIDLGTTYSCVGVWQHDRVEIIANDQGNRTTPSYVAFTDTERLIGDAAKNQVAMNPTNTVFDAKRLIGRRFSSPAVQSSMKLWPSRHLGLGDKPMIVFNYKGEEKQFAAEEISSMVLIKMKEIAEAYLGSTIKNAVVTVPAYFNDSQRQATKDAGVIAGLNVMRIINEPTAAAIAYGLDKKATSSGEKNVLIFDLGGGTFDVSLLTIEEGIFEVKATAGDTHLGGEDFDNRMVNHFVQEFKRKNKKDISGNPRALRRLRTACERAKRTLSSTAQTTIEIDSLFEGIDFTPRSSRARFEELNMDLFRKCMEPVEKCLRDAKMDKSSVHDVVLVGGSTRIPKVQQLQDFFNGKELCKSINPDEAVAYGAAVQAAILSGEGNERSDLLLLDVTPLSLGLETAGGVMTVLIPRNTTIPTKKEQVFSTYSDNQPGVLIQVYEGERARTKDNNLLGKFELSGIPPAPRGVPQITVTFDIDVNNILNVSAEDKTTGQKNKITITNDKGRLSKEEIEKMVQEAEKYKAEDEEVKKKVDAKNALENYAYNMRNTIKDDKIASKLPAEDKKKIEDAVDGAISWLDSNQLAEVEEFEDKMKELEGICNPIIAKMYXGEGAGMGAAAGMDEDAPSGGSGAGPKIEEVD

SEQ ID NO: 10 - protein, 651 aa, Solanum lycopersicum
  Tomato (Q40151), Figure 1
MAGKGEGPAIGIDLGTTYSCVGVWQHDRVEIIANDQGNRTTPSYVAFTDTERLIGDAAKNQVAMNPTNTVFDAKRLIGRRFSDPSVQSDMKLWPFKVIPGPADKPMIVVNYKGEEKQFSAEEISSMVLIKMKEIAEAFLGTTIKNAVVTVPAYFNDSQRQATKDAGTISGLNVMRIINEPTAAAIAYGLDKKISSTGEKTVLIFDLGGGTFDVSLLTIEEGIFEVKATAGDTHLGGEDFDNRMVNHFVQEFKRKHKKDISGNPRALRRLRTACERAKRTLSSTAQTTIEIDSLYEGIDFYTTITRARFEELNMDLFRKCMEPVEKCLRDAKIDKSGVHDIVLVGGSTRIPKVQQLLQDFFNGRELCKSINPDEAVAYGAAVQAAILSGEGNEKVQDLLLLDVTPLSLGLETAGGVMTTLIPRNTTIPTKKEQVFSTYSDNQPGVLIQVYEGERARTRDNNLLGKFELTGIPPAPRGVPQITVCFDIDANGILNVSAEDKTTGQKNKITITNDKGRLSKDEIEKMVQEAEKYKAEDEEHKKKVEAKNALENYAYNMRNTIKDDKIASKLSPDEKKKIEDSVEQAIQWLDGNQLAEADEFEDKMKELESICNPIIAKMYQGRAGPDMAGGMDEDGPSAGASSAGAGPKIEEVD

SEQ ID NO: 11 - protein, 840 aa, Homo sapiens
  HSPA4 (P34932), Figure 2
MSVVGIDLGFQSCYVAVARAGGIETIANEYSDRCTPACISFGPKNRSIGAAAKSQVISNAKNTVQGFKRFHGRAFSDPFVEAEKSNLAYDIVQLPTGLTGIKVTYMEEERNFTTEQVTAMLLSKLKETAESVLKKPVVDCVVSVPCFYTDAERRSVMDATQIAGLNCLRLMNETTAVALAYGIYKQDLPALEEKPRNVVFVDMGHSAYQVSVCAFNRGKLKVLATAFDTTLGGRKFDEVLVNHFCEEFGKKYKLDIKSKIRALLRLSQECEKLKKLMSANASDLPLSIECFMNDVDVSGTMNRGKFLEMCNDLLARVEPPLRSVLEQTKLKKEDIYAVEIVGGATRIPAVKEKISKFFGKELSTTLNADEAVTRGCALQCAILSPAFKVREFSITDVVPYPISLRWNSPAEEGSSDCEVFSKNHAAPFSKVLTFYRKEPFTLEAYYSSPQDLPYPDPAIAQFSVQKVTPQSDGSSSKVKVKVRVNVHGIFSVSSASLVEVHKSEENEEPMETDQNAKEEEKMQVDQEEPHVEEQQQQTPAENKAESEEMETSQAGSKDKKMDQPPQAKKAKVKTSTVDLPIENQLLWQIDREMLNLYIENEGKMIMQDKLEKERNDAKNAVEEYVYEMRDKLSGEYEKFVSEDDRNSFTLKLEDTENWLYEDGEDQPKQVYVDKLAELKNLGQPIKIRFQESEERPKLFEELGKQIQQYMKIISSFKNKEDQYDHLDAADMTKVEKSTNEAMEWMNNKLNLQNKQSLTMDPVVKSKEIEAKIKELTSTCSPIISKPKPKVEPPKEEQKNAEQNGPVDGQGDNPGPQAAEQGTDTAVPSDSDKKLPEMDID

SEQ ID NO: 12 - protein, 679 aa, Homo sapiens
  HSPA9  GRP75 (P38646), Figure 2
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 13 - protein, 654 aa, Homo sapiens
  HSPA5  GRP78 BiP (P11021), Figure 2
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 14 - protein, 367 aa, Homo sapiens
  HSPA7 (P48741), Figure 2
MQAPRELAVGIDLGTTYSCVGVFQQGRVEILANDQGNRTTPSYVAFTDTERLVGDAAKSQAALNPHNTVFDAKRLIGRKFADTTVQSDMKHWPFQVVSEGGKPKVRVCYRGEDKTFYPEEISSMVLSKMKETAEAYLGQPVKHAVITVPTYFSNSQRQATKDAGAIAGLKVLPIINEATAAAIAYGLDRRGAGKRNVLIFDLGGGTFDVSVLSIDAGVFEVKATAGDTHLGGEDFDNRLVNHFMEEFRRKHGKDLSGNKRALRRLRTACERAKRTPSSSTQATLEIDSLFEGVDFYKSITRARFEELCSDLFRSTLEPVEKALRDAKLDKAQIHDFVLGGGLHSHPQGAEVAAGLLQRQGAEQEHQP

SEQ ID NO: 15 - protein, 643 aa, Homo sapiens
  HSPA6 (P17066), Figure 2
MQAPRELAVGIDLGTTYSCVGVFQQGRVEILANDQGNRTTPSYVAFTDTERLVGDAAKSQAALNPHNTVFDAKRLIGRKFADTTVQSDMKHWPFRVVSEGGKPKVRVCYRGEDKTFYPEEISSMVLSKMKETAEAYLGQPVKHAVITVPAYFNDSQRQATKDAGAIAGLNVLRIINEPTAAAIAYGLDRRGAGERNVLIFDLGGGTFDVSVLSIDAGVFEVKATAGDTHLGGEDFDNRLVNHFMEEFRRKHGKDLSGNKRALRRLRTACERAKRTLSSSTQATLEIDSLFEGVDFYTSITRARFEELCSDLFRSTLEPVEKALRDAKLDKAQIHDVVLVGGSTRIPKVQKLLQDFFNGKELNKSINPDEAVAYGAAVQAAVLMGDKCEKVQDLLLLDVAPLSLGLETAGGVMTTLIQRNATIPTKQTQTFTTYSDNQPGVFIQVYEGERAMTKDNNLLGRFELSGIPPAPRGVPQIEVTFDIDANGILSVTATDRSTGKANKITITNDKGRLSKEEVERMVHEAEQYKAEDEAQRDRVAAKNSLEAHVFHVKGSLQEESLRDKIPEEDRRKMQDKCREVLAWLEHNQLAEKEEYEHQKRELEQICRPIFSRLYGGPGVPGGSSCGTQARQGDPSTGPIIEEVD

SEQ ID NO: 16 - protein, 646 aa, Homo sapiens
  HSPA8 (P11142), Figure 2
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 17 - protein, 641 aa, Homo sapiens
  HSPA1L (P34931), Figure 2
MATAKGIAIGIDLGTTYSCVGVFQHGKVEIIANDQGNRTTPSYVAFTDTERLIGDAAKNQVAMNPQNTVFDAKRLIGRKFNDPVVQADMKLWPFQVINEGGKPKVLVSYKGENKAFYPEEISSMVLTKLKETAEAFLGHPVTNAVITVPAYFNDSQRQATKDAGVIAGLNVLRIINEPTAAAIAYGLDKGGQGERHVLIFDLGGGTFDVSILTIDDGIFEVKATAGDTHLGGEDFDNRLVSHFVEEFKRKHKKDISQNKRAVRRLRTACERAKRTLSSSTQANLEIDSLYEGIDFYTSITRARFEELCADLFRGTLEPVEKALRDAKMDKAKIHDIVLVGGSTRIPKVQRLLQDYFNGRDLNKSINPDEAVAYGAAVQAAILMGDKSEKVQDLLLLDVAPLSLGLETAGGVMTALIKRNSTIPTKQTQIFTTYSDNQPGVLIQVYEGERAMTKDNNLLGRFDLTGIPPAPRGVPQIEVTFDIDANGILNVTATDKSTGKVNKITITNDKGRLSKEEIERMVLDAEKYKAEDEVQREKIAAKNALESYAFNMKSVVSDEGLKGKISESDKNKILDKCNELLSWLEVNQLAEKDEFDHKRKELEQMCNPIITKLYQGGCTGPACGTGYVPGRPATGPTIEEVD

SEQ ID NO: 18 - protein, 641 aa, Homo sapiens; identical to SEQ ID NO: 1
  HSPA1B (P0DMV9), Figure 2

SEQ ID NO: 19 - protein, 641 aa, Homo sapiens; identical to SEQ ID NO: 1
  HSPA1A HSP72 (P0DMV8), Figure 2

SEQ ID NO: 20 - protein, 641 aa, Homo sapiens; identical to SEQ ID NO: 1
  Human (P0DMV8), Figure 4A

SEQ ID NO: 21 - protein, 641 aa, Mus musculus; identical to SEQ ID NO: 2
  Mouse (Q61696), Figure 4A

SEQ ID NO: 22 - protein, 641 aa, Homo sapiens; identical to SEQ ID NO: 1
  Human (P0DMV8), Figure 4B

SEQ ID NO: 23 - protein, 641 aa, Mus musculus; identical to SEQ ID NO: 2
  Mouse (Q61696), Figure 4B

SEQ ID NO: 24 - protein, 641 aa, Homo sapiens; identical to SEQ ID NO: 1
  WT human HSP72, Figure 5A

SEQ ID NO: 25 - protein, 641 aa, Artificial sequence
  MOD human HSP72, Figure 5A
MAKAAAIGIDLGTTYSCVGVFQHGKVEIIANDQGNRITPSYVAFTDTERLIGDAAKNQVALQPQQTVFDAKRLIGRKFGDAVVQSDMKHWPFQVINDGDKPKVQVSYKGETKAFYPEEISSMVLTKMKEIAEAYLGYPVTNAVITVPAYFNDAQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLQRSIQPDEAVAYGAAVQAAILMGDKSENVQDLLLLDVAPLSLGLETAGGVMTALIKRNAIIPTKQTQIFTTYSDNQPGVLIQVYEGERAMTKDNQLLGRFELSGIPPAPRGVPQIEVTFDIDANGILHVTATDKSTGKANKITITNDKGRLSKEEIERMVQEAEKYKAEDEVQRERVSAKNALESYAFNMKSAVEDEGLKGKISEADKKKVLDKCQEVISWLDAQTLAEKDEFEHKRKELEQVCQPIISGLYQGAGGPGPGGFGAQGPKGGSGSGPTIEEVD

SEQ ID NO: 26 - protein, 641 aa, Homo sapiens; identical to SEQ ID NO: 1
  WT human HSP72, Figure 5B

SEQ ID NO: 27 - protein, 641 aa, Artificial sequence
  MOD human HSP72, Figure 5B
MAKAAAIGIDLGTTYSCVGVFQHGKVEIIANDQGNRITPSYVAFTDTERLIGDAAKNQVALNPQNTVFDAKRLIGRKFGDAVVQSDMKHWPFQVINDGDKPKVQVSYKGETKAFYPEEISSMVLTKMKEIAEAYLGYPVTNAVITVPAYFNDAQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLQRSINPDEAVAYGAAVQAAILMGDKSENVQDLLLLDVAPLSLGLETAGGVMTALIKRNSIIPTKQTQIFTTYSDNQPGVLIQVYEGERAMTKDNNLLGRFELSGIPPAPRGVPQIEVTFDIDANGILHVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 28 - protein, 641 aa, Homo sapiens; identical to SEQ ID NO: 1
  human HSP72, Figure 5C

SEQ ID NO: 29 - protein, 641 aa, Artificial sequence
  MOD human HSP72, Figure 5C
MAKAAAIGIDLGTTYSCVGVFQHGKVEIIANDQGNRITPSYVAFTDTERLIGDAAKNQVALNPQNTVFDAKRLIGRKFGDAVVQSDMKHWPFQVINDGDKPKVQVSYKGETKAFYPEEISSMVLTKMKEIAEAYLGYPVTNAVITVPAYFNDAQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLQRSINPDEAVAYGAAVQAAILMGDKSENVQDLLLLDVAPLSLGLETAGGVMTALIKRNSIIPTKQTQIFTTYSDNQPGVLIQVYEGERAMTKDNNLLGRFELSGIPPAPRGVPQIEVTFDIDANGILHVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCSPIISGLYQGAGGPGPGGFGAQGPKGGSGSGPTIEEVD

SEQ ID NO: 30 - protein, 205 aa, Homo sapiens
  Human Hsp27 (P04792), Figure 6
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 31 - protein, 209 aa, Mus musculus
  Mouse Hsp27 (P14602), Figure 6
MTERRVPFSLLRSPSWEPFRDWYPAHSRLFDQAFGVPRLPDEWSQWFSAAGWPGYVRPLPAATAEGPAAVTLAAPAFSRALNRQLSSGVSEIRQTADRWRVSLDVNHFAPEELTVKTKEGVVEITGKHEERQDEHGYISRCFTRKYTLPPGVDPTLVSSSLSPEGTLTVEAPLPKAVTQSAEITIPVTFEARAQIGGPEAGKSEQSGAK

SEQ ID NO: 32 - protein, 340 aa, Homo sapiens
  Human Hsp40 (P25685), Figure 6
MGKDYYQTLGLARGASDEEIKRAYRRQALRYHPDKNKEPGAEEKFKEIAEAYDVLSDPRKREIFDRYGEEGLKGSGPSGGSGGGANGTSFSYTFHGDPHAMFAEFFGGRNPFDTFFGQRNGEEGMDIDDPFSGFPMGMGGFTNVNFGRSRSAQEPARKKQDPPVTHDLRVSLEEIYSGCTKKMKISHKRLNPDGKSIRNEDKILTIEVKKGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLIIEFEVIFPERIPQTSRTVLEQVLPI

SEQ ID NO: 33 - protein, 340 aa, Mus musculus
  Mouse Hsp40 (Q9QYJ3), Figure 6
MGKDYYQTLGLARGASDDEIKRAYRRQALRYHPDKNKEPGAEEKFKEIAEAYDVLSDPRKREIFDRYGEEGLKGGSPSGGSSGGANGTSFSYTFHGDPHAMFAEFFGGRNPFDTFFGQRNGEEGMDIDDTFSSFPMGMGGFTNMNFGRSRPSQEPTRKKQDPPVTHDLRVSLEEIYSGCTKKMKISHKRLNPDGKSIRNEDKILTIEVKRGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLVIEFEVIFPERIPVSSRTILEQVLPI

SEQ ID NO: 34 - protein, 573 aa, Homo sapiens
  Human Hsp60 (P10809), Figure 6
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 35 - protein, 573 aa, Mus musculus
  Mouse Hsp60 (P63038), Figure 6
MLRLPTVLRQMRPVSRALAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKDIGNIISDAMKKVGRKGVITVKDGKTLNDELEIIEGMKFDRGYISPYFINTSKGQKCEFQDAYVLLSEKKISSVQSIVPALEIANAHRKPLVIIAEDVDGEALSTLVLNRLKVGLQVVAVKAPGFGDNRKNQLKDMAIATGGAVFGEEGLNLNLEDVQAHDLGKVGEVIVTKDDAMLLKGKGDKAHIEKRIQEITEQLDITTSEYEKEKLNERLAKLSDGVAVLKVGGTSDVEVNEKKDRVTDALNATRAAVEEGIVLGGGCALLRCIPALDSLKPANEDQKIGIEIIKRALKIPAMTIAKNAGVEGSLIVEKILQSSSEVGYDAMLGDFVNMVEKGIIDPTKVVRTALLDAAGVASLLTTAEAVVTEIPKEEKDPGMGAMGGMGGGMGGGMF

SEQ ID NO: 36 - protein, 709 aa, Saccharomyces cerevisiae [sequence omitted]

SEQ ID NO: 37 - protein, 728 aa, Gallus gallus
  Chicken HSP90A (P11501), Figure 6
MPEAVQTQDQPMEEEVETFAFQAEIAQLMSLIINTFYSNKEIFLRELISNSSDALDKIRYESLTDPSKLDSGKDLKINLIPNKHDRTLTIVDTGIGMTKADLVNNLGTIAKSGTKAFMEALQAGADISMIGQFGVGSYSAYLVAEKVTVITKHNDDEQYAWESSAGGSFTVRLDNGEPLGRGTKVILHLKEDQTEYLEERRIKEIVKKHSQFIGYPIRLFVEKERDKEVSDDEAEEKEEEKEEKEEKTEDKPEIEDVGSDEEEEKKDGDKKKKKKIKEKYIDEEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFMRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHVYYITGETKDQVANSAFVERLRKHGLEVIYMIEPIDEYCVQQLKEFEGKTLVSVTKEGLELPEDEEEKKKQEEKKAKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDTAAEEASPAVTEEMPPLEGDDDTSRMEEVD

SEQ ID NO: 38 - protein, 732 aa, Homo sapiens
  Human Hsp90 (P07900), Figure 6
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 39 - protein, 733 aa, Mus musculus
  Mouse Hsp90 (P07901), Figure 6
MPEETQTQDQPMEEEEVETFAFQAEIAQLMSLIINTFYSNKEIFLRELISNSSDALDKIRYESLTDPSKLDSGKELHINLIPSKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEEKEEEKEKEEKESDDKPEIEDVGSDEEEEEKKDGDKKKKKKIKEKYIDQEELNKTKPIWTRNPDDITNEEYGEFYKSLTNDWEEHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYFITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTVDDTSAAVTEEMPPLEGDDDTSRMEEVD

SEQ ID NO: 40 - protein, 776 aa, Caenorhabditis elegans
  C. elegans Hsp110 (Q05036), Figure 6
MSVLGFDIGNLNCYIGVARQGGIEVITNDYSLHATPACVSFGPKDRSMGVAARQAVTTNIKNTVINFKHLIGRKFSDPVAQRFIPFIPCKVVKLPNDDIGVQVSYLGEPHTFTPEQVLAALLTKLRTIVESQLSDVKKVSDCVLAVPSYFTDVQRRAVLSAIQYAGLNSLRIVNETTAIALAYGIYKQDLPEEDAKSRNVVFLDIGHSSTQASLVAFNRGKLQMVNTSYDLESGGIWFDALIREHFRKEFKTKYGIDAATSPRPWLRLLDECERVKKQMSANQTPIPLNIECFMEDKDVTGKMQRQEFEDLAAPIFNRIKQVLINLFADGVSIKPEEIDEIEIVGGSSRIPMIREIVKDLFGKEPKTTMNQDEAVARGAAMQCAILSPTFRVREFAIKDTQPYRIRLSWNSTGENGGENDVFSPRDEVPFSKLVSLLRSGPFNVEAHYAQPNVVPHNQVHIGSWKVNGARPGADGGNQKVKVKVRVNPDGIFTIASATMYEPRIVEEVPAEAMEVDGDAKTEAPAEPLEPVKKTKLVPVDLEVIESIPVSYDVQKFHNLELQMQESDAREKAKADAKNSLEEYVYEMRDKVSDQYAEFITPAAADEFRSVLTSTEDWLYDEGEDAERDVYEKRLSELKAVGTPVVERYRESETRKPAFDSFDQSIMRVRKAYEDYANGGPTYAHLDSKEMEKVINAIEDKKKWLDEARHKQETRSKTDAPVVFTEEILQNKNVFENVVNPILNKKKPAAPAPPKKEEPQPAAGDQPQSQPGEMDVD

SEQ ID NO: 41 - protein, 858 aa, Homo sapiens
  Human Hsp105 (Q92598), Figure 6
MSVVGLDVGSQSCYIAVARAGGIETIANEFSDRCTPSVISFGSKNRTIGVAAKNQQITHANNTVSNFKRFHGRAFNDPFIQKEKENLSYDLVPLKNGGVGIKVMYMGEEHLFSVEQITAMLLTKLKETAENSLKKPVTDCVISVPSFFTDAERRSVLDAAQIVGLNCLRLMNDMTAVALNYGIYKQDLPSLDEKPRIVVFVDMGHSAFQVSACAFNKGKLKVLGTAFDPFLGGKNFDEKLVEHFCAEFKTKYKLDAKSKIRALLRLYQECEKLKKLMSSNSTDLPLNIECFMNDKDVSGKMNRSQFEELCAELLQKIEVPLYSLLEQTHLKVEDVSAVEIVGGATRIPAVKERIAKFFGKDISTTLNADEAVARGCALQCAILSPAFKVREFSVTDAVPFPISLIWNHDSEDTEGVHEVFSRNHAAPFSKVLTFLRRGPFELEAFYSDPQGVPYPEAKIGRFVVQNVSAQKDGEKSRVKVKVRVNTHGIFTISTASMVEKVPTEENEMSSEADMECLNQRPPENPDTDKNVQQDNSEAGTQPQVQTDAQQTSQSPPSPELTSEENKIPDADKANEKKVDQPPEAKKPKIKVVNVELPIEANLVWQLGKDLLNMYIETEGKMIMQDKLEKERNDAKNAVEEYVYEFRDKLCGPYEKFICEQDHQNFLRLLTETEDWLYEEGEDQAKQAYVDKLEELMKIGTPVKVRFQEAEERPKMFEELGQRLQHYAKIAADFRNKDEKYNHIDESEMKKVEKSVNEVMEWMNNVMNAQAKKSLDQDPVVRAQEIKTKIKELNNTCEPVVTQPKPKIESPKLERTPNGPNIDKKEEDLEDKNNFGAEPPHQNGECYPNEKNSVNMDLD

SEQ ID NO: 42 - protein, 858 aa, Mus musculus
  Mouse Hsp105 (Q61699), Figure 6
MSVVGLDVGSQSCYIAVARAGGIETIANEFSDRCTPSVISFGSKNRTIGVAAKNQQITHANNTVSSFKRFHGRAFNDPFIQKEKENLSYDLVPMKNGGVGIKVMYMDEEHFFSVEQITAMLLTKLKETAENNLKKPVTDCVISVPSFFTDAERRSVLDAAQIVGLNCLRLMNDMTAVALNYGIYKQDLPNAEEKPRVVVFVDMGHSSFQVSACAFNKGKLKVLGTAFDPFLGGKNFDEKLVEHFCAEFKTKYKLDAKSKIRALLRLHQECEKLKKLMSSNSTDLPLNIECFMNDKDVSGKMNRSQFEELCAELLQKIEVPLHSLMAQTQLKAEDVSAIEIVGGATRIPAVKERIAKFFGKDVSTTLNADEAVARGCALQCAILSPAFKVREFSVTDAVPFPISLVWNHDSEETEGVHEVFSRNHAAPFSKVLTFLRRGPFELEAFYSDPQGVPYPEAKIGRFVVQNVSAQKDGEKSRVKVKVRVNTHGIFTISTASMVEKVPTEEEDGSSLEADMECPNQRPTESSDVDKNIQQDNSEAGTQPQVQTDGQQTSQSPPSPELTSEESKTPDADKANEKKVDQPPEAKKPKIKVVNVELPVEANLVWQLGRDLLNMYIETEGKMIMQDKLEKERNDAKNAVEECVYEFRDKLCGPYEKFICEQEHEKFLRLLTETEDWLYEEGEDQAKQAYIDKLEELMKMGTPVKVRFQEAEERPKVLEELGQRLQHYAKIAADFRGKDEKYNHIDESEMKKVEKSVNEVMEWMNNVMNAQAKRSLDQDPVVRTHEIRAKVKELNNVCEPVVTQPKPKIESPKLERTPNGPNIDKKEDLEGKNNLGAEAPHQNGECHPNEKGSVNMDLD

SEQ ID NO: 43 - protein, 640 aa, Artificial sequence
  002′s Human Hsp72 sequence, Figure 17A
AKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 44 - protein, 640 aa, Artificial sequence
  003′s Human Hsp72 sequence, Figure 17B
AKAAAIGIDLGTTYSCVGVFQHGKVEIIANDQGNRITPSYVAFTDTERLIGDAAKNQVALNPQNTVFDAKRLIGRKFGDAVVQSDMKHWPFQVINDGDKPKVQVSYKGETKAFYPEEISSMVLTKMKEIAEAYLGYPVTNAVITVPAYFNDAQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLQRSINPDEAVAYGAAVQAAILMGDKSENVQDLLLLDVAPLSLGLETAGGVMTALIKRNSIIPTKQTQIFTTYSDNQPGVLIQVYEGERAMTKDNNLLGRFELSGIPPAPRGVPQIEVTFDIDANGILHVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 45 - DNA, 2751 bp, Artificial sequence
  Pichia Codon Optimized RBB002, Figure 18A
atgcaccatcaccatcaccatgatattgttttgactcaatctccagctagtttagctgtttctttgggtcaaagagctactatttcttgtagagcttccaaatctgtttcaacatcttcttattcttacatgcattggtatcaacagaaaccaggtcaacctccaaaattgctgatcaagtatgcttcttatttggaatctggagttcctgcaagattttctggatctggttcgggaactgattttactttgaacattcatccagttgaagaagaagatgctgctacttattactgtcagcattctagagagtttccttggacttttggtggtggaaccaaattggaaataaaaagagctgatgctgctccaggtggtggtggatctggtggtggtggatctggtggtggtggctctgaagttcaattggttgaatctggtggtggtttggtcaaaccaggtggatctaggaagctttcttgtgctgcttctggattcactttttccaactatggaatgcattgggttcgtcaagctccagaaaagggtttggaatgggttgcttacatttcttctggatcttctactatttattatgctgatactgtcaaaggaagattcaccatttcaagagataatgccaagaacactctttttttgcaaatgacttctttgagatcagaagatactgctatgtactattgtgccagacggggtttattgttggattattggggtcaaggaaccactttgactgtttcttctgcttcgactaaaggtccttctgtttttccattggctccttctttggaatcttctggatccatggcaaaagctgctgctattggaattgatttgggaaccacttactcatgtgttggtgtttttcaacacggaaaagttgaaattattgccaatgatcaaggaaacagaaccactccttcttatgtagctttcactgatactgaaagattgattggtgatgctgccaagaatcaagttgctttgaatcctcaaaacactgtttttgatgccaagagattgattggaagaaagtttggtgatcctgtcgtccaatctgatatgaaacattggccttttcaagttatcaatgatggtgacaaaccaaaggtccaagtttcttacaaaggtgaaaccaaagcattttatccagaagaaatttcttcaatggttttgaccaaaatgaaagaaattgctgaagcttatttgggatatccagttaccaatgccgttattactgttcctgcttacttcaatgattctcaaagacaagctaccaaagatgctggtgtcattgctggtttgaatgttttgagaatcatcaatgaaccaactgctgctgctattgcttacggtttggatagaactggaaaaggtgaaagaaatgttttgatttttgatttgggtggtggaacttttgatgtttccattttgaccattgatgatggtatttttgaagtcaaagctacggcaggtgatactcatttgggtggtgaagattttgacaacagattggtcaatcattttgtcgaggagttcaaaagaaaacacaagaaagatatttctcaaaataaaagagctgttagaagattgagaactgcttgtgaaagagccaagagaactttgtcttcttctactcaagcttctttggagattgattctttgtttgaaggaattgatttttacacttcaatcaccagagctagattcgaagaactttgttctgatcttttcagatctactttggaaccagttgaaaaggctttgcgagatgccaaattggataaagctcaaattcatgatttggttttggttggaggatctactcgcatccccaaagttcaaaaacttttgcaagattttttcaatggaagagatttgaacaaatctataaatccagatgaagctgttgcttatggtgctgctgttcaagctgctattttgatgggggataaatctgaaaatgttcaagatttgttgttgttggatgttgctcctttgtctttgggacttgaaactgctggtggtgtcatgactgctttgatcaaaagaaactctactattccaacaaaacaaactcaaattttcaccacttactctgataaccaaccaggtgttttgattcaagtgtacgaaggtgaaagagctatgaccaaagacaacaatttgttgggaagatttgaattgtcgggcattcctccagctccaagaggtgttcctcaaattgaagtcacttttgatattgatgctaatggtattttgaatgtcactgctactgataaatctactggaaaagccaacaagatcacaattaccaatgataaaggaagattgtccaaagaagaaattgaaagaatggttcaagaagccgaaaagtacaaagctgaagatgaagttcaaagagaaagagtttctgccaagaatgctcttgagtcttatgctttcaacatgaaatctgctgttgaagatgaaggtttgaaaggaaagatttcagaagctgacaagaagaaagttttggataaatgtcaagaagttatttcttggttagatgctaacactttggctgaaaaagatgagtttgaacacaaaagaaaagaattggaacaagtgtgtaacccaatcatttctggtctttatcaaggtgctggtggaccaggtccaggtggattcggcgctcaaggtccaaaaggtggatctggatctggaccaaccattgaagaagttgactaa

SEQ ID NO: 46 - DNA, 2751 bp, Artificial sequence
  Pichia Codon Optimized RBB003, Figure 18B
atgcaccatcaccatcaccatgatattgttttgactcaatctccagctagtttagctgtttctttgggtcaaagagctactatttcttgtagagcttccaaatctgtttcaacatcttcttattcttacatgcattggtatcaacagaaaccaggtcaacctccaaaattgctgatcaagtatgcttcttatttggaatctggagttcctgcaagattttctggatctggttcgggaactgattttactttgaacattcatccagttgaagaagaagatgctgctacttattactgtcagcattctagagagtttccttggacttttggtggtggaaccaaattggaaataaaaagagctgatgctgctccaggtggtggtggatctggtggtggtggatctggtggtggtggctctgaagttcaattggttgaatctggtggtggtttggtcaaaccaggtggatctaggaagctttcttgtgctgcttctggattcactttttccaactatggaatgcattgggttcgtcaagctccagaaaagggtttggaatgggttgcttacatttcttctggatcttctactatttattatgctgatactgtcaaaggaagattcaccatttcaagagataatgccaagaacactctttttttgcaaatgacttctttgagatcagaagatactgctatgtactattgtgccagacggggtttattgttggattattggggtcaaggaaccactttgactgtttcttctgcttcgactaaaggtccttctgtttttccattggctccttctttggaatcttctggatccatggcaaaagctgctgctattggaattgatttgggaaccacttactcatgtgttggtgtttttcaacacggaaaagttgaaattattgccaatgatcaaggaaacagaatcactccttcttatgtagcattcactgatactgaaagattgattggtgatgctgccaagaatcaagttgctttgaaccctcaaaacactgtttttgatgccaagagattgattggaagaaagtttggtgatcctgtagttcaatctgatatgaaacattggccttttcaagttatcaatgatggtgacaaaccaaaggtccaagtttcttacaaaggtgaaaccaaagcattttatccagaagaaatttcttcaatggtcttgaccaaaatgaaagaaattgctgaagcttatttgggatatccagttaccaatgcagtcattactgttcctgcttacttcaatgatgctcaaagacaagctaccaaagatgctggtgtaattgctggtttgaatgttttgagaatcatcaatgaaccaactgctgctgctattgcgtatggtttggatagaactggaaaaggtgaaagaaatgttttgatttttgatttgggtggtgggacttttgatgtttccattttgaccattgatgatggtatttttgaagtcaaagctaccgccggtgatactcatttgggtggtgaagattttgacaacagattggtcaatcattttgttgaagagttcaaaagaaaacacaagaaagatatttctcaaaataaaagagctgttagaaggttaagaactgcttgtgaaagagccaagagaactttgtcttcttctactcaagcttctttagaaattgattctttgtttgaaggaattgatttttacacttcaatcaccagagctagattcgaagaactttgttctgatcttttcagatctactttggaaccagttgaaaaggctttgagagacgccaaattggataaagctcaaattcatgatttggttttggttggaggatctactaggatacccaaagttcaaaaacttttgcaagattttttcaatggaagagatttgcaaagaagcataaatccagatgaagctgttgcttatggtgctgctgttcaagctgctattttgatgggtgacaaatctgaaaatgttcaagatttgttgttgttggatgttgctcctttgtctttgggcctggaaactgctggtggtgtcatgactgctttgatcaaaagaaactccatcattccaactaagcaaactcaaattttcaccacttactctgataaccaaccaggtgttttgattcaagtttacgaaggtgaaagagctatgaccaaagacaacaatttgttgggaagatttgaattgtcgggtattcctccagctccaagaggtgttcctcaaattgaagtcacttttgatattgatgctaatggtattttgcatgtcactgctactgataaatctactggaaaagccaacaagatcacaataaccaatgataaaggaagattgtccaaagaagaaattgaaagaatggttcaagaagcagaaaagtacaaagctgaagatgaagttcaaagagaaagagtttctgccaagaatgctttggaatcttatgctttcaacatgaaatctgctgttgaagatgaaggtttgaaaggaaagatctccgaagctgacaagaagaaagttttggataaatgtcaagaagttatttcttggttagatgcgaacactttggctgaaaaagatgagtttgaacacaaaagaaaagaattggaacaagtttgtaacccaatcatttctggtctttatcaaggtgctggtggaccaggtccaggtggattcggtgctcaaggtccaaaaggtggatctggatctggaccaaccattgaagaagttgattaa

SEQ ID NO: 47 - DNA, 2760 bp, Artificial sequence
  Baculovirus (Insect) Codon Optimized RBB002, Figure 19A
atgcaccaccatcatcatcatgacatagtgctgacccaatcccccgcctctctcgctgtatcacttggccaaagggccacaatttcttgtcgggcttcaaagtctgtctccacaagctcatattcatatatgcattggtatcaacagaagcccggacagccgcccaaactgctcatcaaatacgcaagctacctcgagtcaggagtcccagctagattcagtggatcaggttccggcaccgacttcactctgaatatccatcccgtggaggaggaggatgctgcaacgtattactgccagcattcacgagagttcccttggactttcggcggtggtaccaaactggagatcaaaagagccgatgcagcgcccggggggggaggttcaggcggtggagggtctggcggagggggctcagaggtgcagctcgtcgaatctggaggcggcctcgtgaagcctgggggtagtaggaaactctcttgtgccgcttccggattcaccttctctaactatggcatgcattgggtccgccaagccccagagaaagggctggaatgggtcgcctacattagtagtgggagctccaccatctattatgccgatactgtgaaaggacggttcaccatctcacgagataatgcaaagaacacactcttcctccaaatgacctcactgcggagcgaggataccgctatgtactattgtgcgaggagagggttgctcctcgattattggggacaggggaccaccctgacggtgtcatcagccagcacgaaaggtcccagcgttttcccactcgccccatcacttgagagttcaggatcaatggcaaaggcagcagcaattggcattgatctcggaaccacatacagttgtgttggagtgtttcagcacggaaaggttgagattattgcaaacgaccagggcaacaggaccacaccttcttatgttgctttcactgataccgaaagactgatcggcgatgccgccaaaaatcaagtggcgctgaatccacagaataccgtgttcgacgccaagcgcctgatcggtcgaaagttcggggaccctgtggtacagtctgacatgaagcattggccatttcaagtcattaacgatggggacaagccaaaagtccaggtttcatataagggggagactaaggccttctatcctgaggagatatcaagcatggtgctcaccaaaatgaaagagatcgctgaggcttatctggggtatccagtgaccaatgctgtgataaccgttccggcctactttaatgacagccagcgccaggctacgaaggacgctggtgtaattgctggactgaacgtccttagaattatcaatgagcccacagctgccgcaatcgcttacggactggaccggaccggaaagggagagcgaaatgtgctgatcttcgacctcggcggaggaacctttgatgtatcaattttgactatcgacgacgggatttttgaggtcaaagctaccgccggcgacacgcatctgggcggagaagatttcgataatcgactggttaaccatttcgtggaggaattcaagcggaagcacaaaaaggacattagccagaacaagagggccgtgcggcgactgcggactgcttgcgagcgggctaaaaggactctgtcatcaagtacacaggcctcactggagattgactcactgttcgagggcattgacttctatacaagcattaccagagccagattcgaggaactgtgcagtgatttgtttagatcaaccctcgagccggtagagaaggctctcagagatgccaaattggataaagcacagatacacgacctcgttctcgtgggcgggagcaccagaataccaaaagtgcagaagctgctgcaggatttcttcaacggcagagatcttaacaaatcaatcaatcccgacgaagccgtggcatacggtgctgccgtgcaagccgctatcctcatgggagacaaatcagaaaacgttcaggatctgctcctccttgacgttgcccccttgagcctcggactcgaaaccgctggcggagtgatgacagctctcatcaaaagaaactcaaccattcccacgaaacagacacagatctttactacatatagcgataaccagcctggtgtcctcatacaagtatatgaaggagagagagcgatgacaaaggataacaatctcttgggacgcttcgagctcagtggaattcctcccgcgccaaggggggtcccccagattgaggttacattcgatatagatgccaacggaattctcaacgtgaccgcgaccgacaaatctaccggaaaggccaataagattaccatcaccaatgacaagggccgactgtcaaaagaagagattgaacggatggtacaggaggctgagaagtacaaagccgaggacgaagtgcagagagagcgcgtttccgcgaagaacgctctcgaatcctatgctttcaacatgaagagtgcagtggaagatgaggggcttaaagggaagatatccgaggctgacaagaagaaggttctcgacaaatgccaagaggtcatttcctggctggatgcaaatacgctggccgagaaggacgagtttgaacataagcgaaaagagctggaacaagtctgcaatcccatcatatctggcctgtatcagggcgccggaggaccgggtccaggcggatttggagctcagggaccaaaaggaggctcaggctccgggccaaccatagaggaggtggactgataaaagctt

SEQ ID NO: 48 - DNA, 2760 bp, Artificial sequence
  Baculovirus (Insect) Codon Optimized RBB003, Figure 19B
atgcaccaccatcatcatcatgacatagtgctgacccaatcccccgcctctctcgctgtatcacttggccaaagggccacaatttcttgtcgggcttcaaagtctgtctccacaagctcatattcatatatgcattggtatcaacagaagcccggacagccgcccaaactgctcatcaaatacgcaagctacctcgagtcaggagtcccagctagattcagtggatcaggttccggcaccgacttcactctgaatatccatcccgtggaggaggaggatgctgcaacgtattactgccagcattcacgagagttcccttggactttcggcggtggtaccaaactggagatcaaaagagccgatgcagcgcccggggggggaggttcaggcggtggagggtctggcggagggggctcagaggtgcagctcgtcgaatctggaggcggcctcgtgaagcctgggggtagtaggaaactctcttgtgccgcttccggattcaccttctctaactatggcatgcattgggtccgccaagccccagagaaagggctggaatgggtcgcctacattagtagtgggagctccaccatctattatgccgatactgtgaaaggacggttcaccatctcacgagataatgcaaagaacacactcttcctccaaatgacctcactgcggagcgaggataccgctatgtactattgtgcgaggagagggttgctcctcgattattggggacaggggaccaccctgacggtgtcatcagccagcacgaaaggtcccagcgttttcccactcgccccatcacttgagagttcaggatcaatggcaaaggcagcagcaattggcattgatctcggaaccacatacagttgtgttggagtgtttcagcacggaaaggttgagattattgcaaacgaccagggcaacaggattacaccttcttatgttgctttcactgataccgaaagactgatcggcgatgccgccaaaaatcaagtggcgctgaatccacagaataccgtgttcgacgccaagcgcctgatcggtcgaaagttcggggaccctgtggtacagtctgacatgaagcattggccatttcaagtcattaacgatggggacaagccaaaagtccaggtttcatataagggggagactaaggccttctatcctgaggagatatcaagcatggtgctcaccaaaatgaaagagatcgctgaggcttatctggggtatccagtgaccaatgctgtgataaccgttccggcctactttaatgacgcacagcgccaggctacgaaggacgctggtgtaattgctggactgaacgtccttagaattatcaatgagcccacagctgccgcaatcgcttacggactggaccggaccggaaagggagagcgaaatgtgctgatcttcgacctcggcggaggaacctttgatgtatcaattttgactatcgacgacgggatttttgaggtcaaagctaccgccggcgacacgcatctgggcggagaagatttcgataatcgactggttaaccatttcgtggaggaattcaagcggaagcacaaaaaggacattagccagaacaagagggccgtgcggcgactgcggactgcttgcgagcgggctaaaaggactctgtcatcaagtacacaggcctcactggagattgactcactgttcgagggcattgacttctatacaagcattaccagagccagattcgaggaactgtgcagtgatttgtttagatcaaccctcgagccggtagagaaggctctcagagatgccaaattggataaagcacagatacacgacctcgttctcgtgggcgggagcaccagaataccaaaagtgcagaagctgctgcaggatttcttcaacggcagagatcttcagcgctcaatcaatcccgacgaagccgtggcatacggtgctgccgtgcaagccgctatcctcatgggagacaaatcagaaaacgttcaggatctgctcctccttgacgttgcccccttgagcctcggactcgaaaccgctggcggagtgatgacagctctcatcaaaagaaactcaatcattcccacgaaacagacacagatctttactacatatagcgataaccagcctggtgtcctcatacaagtatatgaaggagagagagcgatgacaaaggataacaatctcttgggacgcttcgagctcagtggaattcctcccgcgccaaggggggtcccccagattgaggttacattcgatatagatgccaacggaattctccacgtgaccgcgaccgacaaatctaccggaaaggccaataagattaccatcaccaatgacaagggccgactgtcaaaagaagagattgaacggatggtacaggaggctgagaagtacaaagccgaggacgaagtgcagagagagcgcgtttccgcgaagaacgctctcgaatcctatgctttcaacatgaagagtgcagtggaagatgaggggcttaaagggaagatatccgaggctgacaagaagaaggttctcgacaaatgccaagaggtcatttcctggctggatgcaaatacgctggccgagaaggacgagtttgaacataagcgaaaagagctggaacaagtctgcaatcccatcatatctggcctgtatcagggcgccggaggaccgggtccaggcggatttggagctcagggaccaaaaggaggctcaggctccgggccaaccatagaggaggtggactgataaaagctt

SEQ ID NO: 49 - DNA, 2751 bp, Artificial sequence
  Escherichia coli Codon Optimized RBB002, Figure 20A
atgcatcatcatcaccatcatgatattgttctgacccagagtccggcaagcctggcagttagcctgggtcagcgtgcaaccattagctgtcgtgcaagcaaaagcgttagcaccagcagctatagttatatgcattggtatcagcagaaaccgggtcagcctccgaaactgctgatcaaatatgcaagctatctggaaagcggtgttccggcacgttttagcggtagcggtagtggcaccgattttaccctgaacattcatccggttgaagaggaagatgcagcaacctattattgtcagcatagccgtgaatttccgtggacctttggtggtggcaccaaactggaaattaaacgtgcagatgccgcaccgggtggtggtggtagtggtggcggtggttcaggcggtggcggtagcgaagttcagctggttgaatcaggtggtggtctggttaaaccgggtggcagccgtaaactgagctgtgcagcaagcggttttacctttagcaattatggcatgcattgggttcgtcaggcaccggaaaaaggtctggaatgggttgcatatattagcagcggtagcagcaccatctattatgcagataccgttaaaggtcgctttaccattagccgtgataatgcaaaaaataccctgtttctgcagatgaccagcctgcgtagcgaagataccgcaatgtattattgtgcacgtcgtggtctgctgttagattattggggtcagggcaccaccctgaccgttagcagcgcaagcacaaaaggtccgagcgtttttccgctggcaccgagcctggaaagcagcggttcagcaaaagcagcagccattggtattgatctgggtacaacctatagctgtgttggtgtttttcagcatggcaaggttgaaatcattgcaaatgatcagggcaatcgtaccaccccgagctatgttgcatttaccgataccgaacgtctgattggtgatgcagccaaaaatcaggttgcactgaatccgcagaataccgtttttgatgcaaaacgcctgattggccgtaaatttggagatgcagttgttcagagcgatatgaaacattggccgtttcaggttattaacgatggcgataaaccgaaagttcaggtgagctataaaggtgaaaccaaagcattttatccggaagaaattagcagcatggttctgaccaaaatgaaagaaattgcagaagcctatctgggttatccggttaccaatgccgttattaccgttccggcatatttcaatgatagccagcgtcaggcaaccaaagatgccggtgttattgcaggtctgaatgttctgcgtattatcaatgaaccgacagcagccgcaattgcctatggtctggatcgtaccggtaaaggcgaacgtaatgttctgattttcgatctgggtggtggaacctttgatgttagcattctgaccattgatgatggcatcttcgaagttaaagcaaccgcaggcgatacccatctgggaggtgaagattttgataatcgtctggtgaaccactttgtggaagagtttaaacgcaaacataaaaaagatattagccagaacaaacgtgcggttcgtcgtctgcgtaccgcatgtgaacgtgcaaaacgtaccctgagcagcagtacccaggcaagtctggaaattgatagcctgtttgaaggcatcgatttctataccagcattacccgtgcacgttttgaagaactgtgtagcgacctgtttcgtagcaccctggaaccggttgaaaaagcactgcgtgatgcgaaactggataaagcacagattcatgatctggtactggtgggtggtagcacccgtattccgaaagtgcaaaaactgctgcaggattttttcaatggtcgcgatctgaacaaaagcattaatccggatgaagcagttgcgtatggtgcagccgttcaggcagcaattctgatgggtgataaaagcgaaaatgtgcaggacctgctgttactggatgttgcaccgctgagtctgggtctggaaacagccggtggtgttatgaccgcactgattaaacgtaatagcaccattccgaccaaacagacacagatttttaccacctatagcgataatcagcctggtgtgctgattcaggtttatgaaggtgaacgcgcaatgaccaaagataataatctgctgggtcgttttgaactgagcggtattccgcctgcaccgcgtggtgttccgcagattgaagttacctttgatattgatgccaacggcattctgaatgtgaccgcaaccgataaaagcaccggcaaagcaaacaaaattaccatcacgaatgataaaggccgtctgagcaaagaagaaattgaacgtatggttcaagaggccgaaaaatacaaagccgaagatgaagtgcagcgtgaacgtgttagcgcaaaaaatgcactggaaagctatgcgtttaacatgaaaagcgcagttgaagatgagggtctgaaaggcaaaattagcgaagccgataaaaagaaagtgctggataaatgccaagaagtgattagctggctggatgccaataccctggcagaaaaagatgaatttgaacacaaacgcaaagaactggaacaggtttgcaatccgattattagcggtctgtatcagggtgccggtggtccgggtccgggtggttttggtgcacagggtccgaaaggtggtagcggttctggtccgacaattgaagaagttgattaataa

SEQ ID NO: 50 - protein, 915 aa, Artificial sequence
  Peptide sequence of the RBB002 (Cytoplasmic), Figure 20A
MHHHHHHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPSLESSGSAKAAAIGIDLGTTYSCVGVFQHGKVEIIANDQGNRTTPSYVAFTDTERLIGDAAKNQVALNPQNTVFDAKRLIGRKFGDAVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 51 - DNA, 2751 bp, Artificial sequence
  Escherichia coli Codon Optimized RBB003, Figure 20B
atgcatcatcatcaccatcatgatattgttctgacccagagtccggcaagcctggcagttagcctgggtcagcgtgcaaccattagctgtcgtgcaagcaaaagcgttagcaccagcagctatagttatatgcattggtatcagcagaaaccgggtcagcctccgaaactgctgatcaaatatgcaagctatctggaaagcggtgttccggcacgttttagcggtagcggtagtggcaccgattttaccctgaacattcatccggttgaagaggaagatgcagcaacctattattgtcagcatagccgtgaatttccgtggacctttggtggtggcaccaaactggaaattaaacgtgcagatgccgcaccgggtggtggtggtagtggtggcggtggttcaggcggtggcggtagcgaagttcagctggttgaatcaggtggtggtctggttaaaccgggtggcagccgtaaactgagctgtgcagcaagcggttttacctttagcaattatggcatgcattgggttcgtcaggcaccggaaaaaggtctggaatgggttgcatatattagcagcggtagcagcaccatctattatgcagataccgttaaaggtcgctttaccattagccgtgataatgcaaaaaataccctgtttctgcagatgaccagcctgcgtagcgaagataccgcaatgtattattgtgcacgtcgtggtctgctgttagattattggggtcagggcaccaccctgaccgttagcagcgcaagcacaaaaggtccgagcgtttttccgctggcaccgagcctggaaagcagcggttcagcaaaagcagcagccattggtattgatctgggtacaacctatagctgtgttggtgtttttcagcatggcaaggttgaaatcattgcaaatgatcagggcaatcgtattaccccgagctatgttgcatttaccgataccgaacgtctgattggtgatgcagcaaaaaatcaggttgcactgaatccgcagaataccgtttttgatgcaaaacgcctgattggccgtaaatttggagatgcagttgttcagagcgatatgaaacattggccgtttcaggttattaacgatggcgataaaccgaaagttcaggtgagctataaaggtgaaaccaaagcattttatccggaagaaattagcagcatggttctgaccaaaatgaaagaaattgcagaagcctatctgggttatccggttaccaatgcagttattaccgttccggcatatttcaatgatgcacagcgtcaggcaaccaaagatgccggtgttattgcaggtctgaatgttctgcgtattatcaatgaaccgaccgcagcagcaattgcctatggtctggatcgtaccggtaaaggcgaacgtaatgttctgatttttgatctgggtggtggcacctttgatgttagcattctgaccattgatgatggcatcttcgaagttaaagcaaccgcaggcgatacccatctgggaggtgaagattttgataatcgtctggtgaaccactttgtggaagagtttaaacgcaaacataaaaaagatattagccagaacaaacgtgcagttcgtcgtctgcgtaccgcatgtgaacgtgcaaaacgtaccctgagcagcagcacccaggcaagcctggaaattgatagcctgtttgaaggcattgatttctataccagcattacccgtgcacgttttgaagaactgtgtagcgacctgtttcgtagcaccctggaaccggttgaaaaagcactgcgtgatgccaaactggataaagcacagattcatgatctggttctggttggtggtagcacccgtattccgaaagtgcagaaactgctgcaggattttttcaatggtcgtgatctgcagcgtagcattaatccggatgaagcagttgcgtatggtgcagccgttcaggcagcaattctgatgggtgataaaagcgaaaatgttcaggatctgctgctgttagatgttgcaccgctgagcctgggtctggaaacagccggtggtgttatgaccgcactgattaaacgtaatagcattattccgaccaagcagacccagatttttaccacctatagcgataatcagcctggtgtgctgattcaggtttatgaaggtgaacgcgcaatgaccaaagataataatctgctgggtcgttttgaactgagcggtattccgcctgcaccgcgtggtgttccgcagattgaagttacctttgatattgatgccaacggcattctgcatgttaccgcaaccgataaaagcaccggcaaagcaaacaaaattaccatcacgaatgataaaggtcgcctgagcaaagaagaaattgaacgtatggttcaagaggccgaaaaatacaaagccgaagatgaagttcagcgtgaacgtgttagcgcaaaaaatgcactggaaagctatgcgtttaacatgaaaagcgcagttgaagatgagggtctgaaaggtaaaattagcgaagccgataaaaagaaagtgctggataaatgccaagaagtgattagctggctggatgcaaataccctggcagaaaaagatgaatttgagcacaaacgcaaagaactggaacaggtttgtaatccgattatcagcggtctgtatcagggtgccggtggtccgggtccgggtggttttggtgcacagggtccgaaaggcggtagcggtagtggtccgacaattgaagaagttgattaataa

SEQ ID NO: 52 - protein, 915 aa, Artificial sequence
  Peptide sequence of the RBB003 (Cytoplasmic), Figure 20B
MHHHHHHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPSLESSGSAKAAAIGIDLGTTYSCVGVFQHGKVEIIANDQGNRITPSYVAFTDTERLIGDAAKNQVALNPQNTVFDAKRLIGRKFGDAVVQSDMKHWPFQVINDGDKPKVQVSYKGETKAFYPEEISSMVLTKMKEIAEAYLGYPVTNAVITVPAYFNDAQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLQRSINPDEAVAYGAAVQAAILMGDKSENVQDLLLLDVAPLSLGLETAGGVMTALIKRNSIIPTKQTQIFTTYSDNQPGVLIQVYEGERAMTKDNNLLGRFELSGIPPAPRGVPQIEVTFDIDANGILHVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 53 - DNA, 2805 bp, Artificial sequence
  Mammalian Codon Optimized RBB002, Figure 21A
atgggctggtcctgcatcatcctgtttctggtggccaccgccaccggcgtgcactcccaccaccatcaccaccacgacatcgtgctgacccagagccctgcctccctggccgtgtctctgggccagagagccaccatcagctgccgggcctccaagtccgtgtccacctcctcctactcctacatgcactggtatcagcagaagcccggccagccccccaagctgctgattaagtacgcctcctatctggaatccggcgtgcccgccagattctccggctctggctctggcaccgacttcaccctgaacatccaccccgtggaagaagaggacgctgccacctactactgccagcactccagagagttcccttggaccttcggcggaggaaccaagctggaaatcaagagagccgacgccgctcctggcggaggaggatctggcggcggaggaagtggcgggggaggatctgaggtgcagctggtggaaagcggcggaggactggtgaaacctggcggctccagaaagctgtcctgcgccgcctccggcttcaccttctccaactacggcatgcactgggtgcgacaggcccccgagaagggcctggaatgggtggcctacatctcctccggctcctccaccatctactacgccgacaccgtgaagggccggttcaccatctcccgggacaacgccaagaacaccctgtttctgcagatgacctccctgcggagcgaggacaccgctatgtactactgcgccagacggggcctgctgctggactattggggccagggcaccaccctgaccgtctcctctgcctccaccaagggcccctccgtgttccctctggccccctccctggaatcctccggcagcgccaaggccgctgccatcggcatcgacctgggcaccacctacagctgcgtgggcgtgttccagcacggcaaggtggaaatcattgccaacgaccagggcaaccggaccaccccctcctacgtggccttcaccgacaccgagagactgatcggcgacgccgccaagaaccaggtggccctgaacccccagaacaccgtgttcgacgccaagcggctgatcggccggaagtttggcgacgccgtggtgcagtccgacatgaagcactggccatttcaggtcatcaacgacggcgacaagcccaaggtgcaggtctcctacaagggcgagacaaaggccttctaccccgaagagatctcctccatggtgctgaccaagatgaaggaaatcgccgaggcctacctgggctaccccgtgaccaacgccgtgatcaccgtgcccgcctacttcaacgactcccagcggcaggccaccaaggacgctggcgtgatcgccggcctgaacgtgctgcggatcatcaacgagcccaccgccgctgctatcgcctacggcctggatagaaccggcaagggcgagcggaacgtgctgatcttcgacctgggcggcggcaccttcgacgtgtccatcctgaccatcgacgacggcatcttcgaagtgaaggccaccgctggcgacacccacctgggcggcgaggacttcgacaaccggctggtgaaccacttcgtggaagagttcaagcggaagcacaagaaggacatctcccagaacaagcgggccgtgcggcggctgagaaccgcctgcgagagagccaagcggaccctgtccagctccacccaggccagcctggaaatcgactccctgttcgagggcatcgacttctacacctccatcacccgggccagattcgaggaactgtgctccgacctgttccggtccaccctggaacccgtggaaaaggccctgcgggacgccaagctggacaaggcccagatccacgacctggtgctggtcggcggctccaccagaatccctaaggtgcagaagctgctgcaggacttcttcaacggccgggacctgaacaagtccatcaaccccgacgaggccgtggcctacggcgctgctgtgcaggctgccatcctgatgggcgacaagtccgagaacgtgcaggacctgctgctgctggatgtggcccctctgtccctgggcctggaaaccgctggcggcgtgatgaccgccctgatcaagcggaactccaccatccctaccaagcagacccagatcttcaccacctactccgacaaccagcccggcgtgctgatccaggtgtacgagggcgagagggccatgaccaaggacaacaacctgctgggcagattcgagctgtccggcatcccccctgcccctagaggcgtgccccagatcgaagtgaccttcgacatcgatgccaacggcatcctgaacgtgaccgccaccgacaagtccaccggcaaggccaacaagatcaccatcacaaacgacaagggccggctgtccaaagaagagatcgagcggatggtgcaggaagccgagaagtacaaggccgaggacgaggtgcagcgcgagagagtgtccgctaagaacgccctggaaagctacgccttcaacatgaagtccgctgtggaagatgagggcctgaagggcaagatctccgaggccgacaagaaaaaggtgctggacaagtgccaggaagtgatctcctggctggacgccaacaccctggccgagaaggacgagttcgagcacaagcggaaagaactggaacaagtctgcaaccccatcatctccggcctgtaccagggcgctggcggacctggacctggcggatttggcgctcagggccctaagggcggctccggctccggccctacaatcgaagaggtggactgatag

SEQ ID NO: 54 - protein, 933 aa, Artificial sequence
  Amino acid sequence of RBB002, Figure 21A
MGWSCIILFLVATATGVHSHHHHHHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPSLESSGSAKAAAIGIDLGTTYSCVGVFQHGKVEIIANDQGNRTTPSYVAFTDTERLIGDAAKNQVALNPQNTVFDAKRLIGRKFGDAVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 55 - protein, 19 aa, Artificial sequence
  Signal peptide, Figure 21A
MGWSCIILFLVATATGVHS

SEQ ID NO: 56 - DNA, 2805 bp, Artificial sequence
  Mammalian Codon Optimized RBB003, Figure 21B
atgggctggtcctgcatcatcctgtttctggtggccaccgccaccggcgtgcactcccaccaccatcaccaccacgacatcgtgctgacccagagccctgcctccctggccgtgtctctgggccagagagccaccatcagctgccgggcctccaagtccgtgtccacctcctcctactcctacatgcactggtatcagcagaagcccggccagccccccaagctgctgattaagtacgcctcctatctggaatccggcgtgcccgccagattctccggctctggctctggcaccgacttcaccctgaacatccaccccgtggaagaagaggacgctgccacctactactgccagcactccagagagttcccttggaccttcggcggaggaaccaagctggaaatcaagagagccgacgccgctcctggcggaggaggatctggcggcggaggaagtggcgggggaggatctgaggtgcagctggtggaaagcggcggaggactggtgaaacctggcggctccagaaagctgtcctgcgccgcctccggcttcaccttctccaactacggcatgcactgggtgcgacaggcccccgagaagggcctggaatgggtggcctacatctcctccggctcctccaccatctactacgccgacaccgtgaagggccggttcaccatctcccgggacaacgccaagaacaccctgtttctgcagatgacctccctgcggagcgaggacaccgctatgtactactgcgccagacggggcctgctgctggactattggggccagggcaccaccctgaccgtctcctctgcctccaccaagggcccctccgtgttccctctggccccctccctggaatcctccggcagcgccaaggccgctgccatcggcatcgacctgggcaccacctacagctgcgtgggcgtgttccagcacggcaaggtggaaatcattgccaacgaccagggcaaccggatcaccccctcctacgtggccttcaccgacaccgagagactgatcggcgacgccgccaagaaccaggtggccctgaacccccagaacaccgtgttcgacgccaagcggctgatcggccggaagtttggcgacgccgtggtgcagtccgacatgaagcactggccatttcaggtcatcaacgacggcgacaagcccaaggtgcaggtctcctacaagggcgagacaaaggccttctaccccgaagagatctcctccatggtgctgaccaagatgaaggaaatcgccgaggcctacctgggctaccccgtgaccaacgccgtgatcaccgtgcccgcctacttcaacgacgcccagcggcaggccaccaaggatgctggcgtgatcgccggcctgaacgtgctgcggatcatcaacgagcccaccgccgctgctatcgcctacggcctggatagaaccggcaagggcgagcggaacgtgctgatcttcgacctgggcggcggcaccttcgacgtgtccatcctgaccatcgacgacggcatcttcgaagtgaaggccaccgctggcgacacccacctgggcggcgaggacttcgacaaccggctggtgaaccacttcgtggaagagttcaagcggaagcacaagaaggacatctcccagaacaagcgggccgtgcggcggctgagaaccgcctgcgagagagccaagcggaccctgtccagctccacccaggcctccctggaaatcgactccctgttcgagggcatcgacttctacacctccatcacccgggccagattcgaggaactgtgctccgacctgttccggtccaccctggaacccgtggaaaaggccctgcgggacgccaagctggacaaggcccagatccacgacctggtgctggtcggcggctccacaagaatccctaaggtgcagaagctgctgcaggacttcttcaacggccgggacctgcagcggtccatcaaccctgatgaggccgtggcctacggcgctgccgtgcaggctgctatcctgatgggcgacaagtccgagaacgtgcaggacctgctgctgctggacgtggcccctctgtccctgggcctggaaaccgctggcggcgtgatgaccgccctgatcaagcggaactccatcatccccaccaagcagacccagatcttcaccacctactccgacaaccagcccggcgtgctgatccaggtgtacgagggcgagagggccatgaccaaggacaacaacctgctgggcagattcgagctgtccggcatcccccctgcccctagaggcgtgccccagatcgaagtgaccttcgacatcgatgccaacggcatcctgcacgtgaccgccaccgacaagtccaccggcaaggccaacaagatcaccatcaccaacgacaagggccggctgtccaaagaagagatcgagcggatggtgcaggaagccgagaagtacaaggccgaggacgaggtgcagcgcgagagagtgtctgccaagaacgccctggaatcctacgccttcaacatgaagtccgccgtggaagatgagggcctgaagggcaagatctccgaggccgacaagaaaaaggtgctggacaagtgccaggaagtgatctcctggctggacgccaacaccctggccgagaaggacgagttcgagcacaagcggaaagaactggaacaagtctgcaaccccatcatctccggcctgtaccagggcgctggcggacctggacctggcggatttggcgctcagggccctaagggcggctctggctctggccccaccatcgaagaggtggactgatag

SEQ ID NO: 57 - protein, 933 aa, Artificial sequence
  Amino acid sequence of RBB003, Figure 21B
MGWSCIILFLVATATGVHSHHHHHHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPSLESSGSAKAAAIGIDLGTTYSCVGVFQHGKVEIIANDQGNRITPSYVAFTDTERLIGDAAKNQVALNPQNTVFDAKRLIGRKFGDAVVQSDMKHWPFQVINDGDKPKVQVSYKGETKAFYPEEISSMVLTKMKEIAEAYLGYPVTNAVITVPAYFNDAQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLQRSINPDEAVAYGAAVQAAILMGDKSENVQDLLLLDVAPLSLGLETAGGVMTALIKRNSIIPTKQTQIFTTYSDNQPGVLIQVYEGERAMTKDNNLLGRFELSGIPPAPRGVPQIEVTFDIDANGILHVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 58 - DNA, 2787 bp, Artificial sequence
  Mammalian Codon Optimized RBB004, Figure 21C
atgggctggtcctgcatcatcctgtttctggtggccaccgccaccggcgtgcactccgacatcgtgctgacccagagccctgcctccctggccgtgtctctgggccagagagccaccatcagctgccgggcctccaagtccgtgtccacctcctcctactcctacatgcactggtatcagcagaagcccggccagccccccaagctgctgattaagtacgcctcctatctggaatccggcgtgcccgccagattctccggctctggctctggcaccgacttcaccctgaacatccaccccgtggaagaagaggacgctgccacctactactgccagcactccagagagttcccttggaccttcggcggaggaaccaagctggaaatcaagagagccgacgccgctcctggcggaggaggatctggcggcggaggaagtggcgggggaggatctgaggtgcagctggtggaaagcggcggaggactggtgaaacctggcggctccagaaagctgtcctgcgccgcctccggcttcaccttctccaactacggcatgcactgggtgcgacaggcccccgagaagggcctggaatgggtggcctacatctcctccggctcctccaccatctactacgccgacaccgtgaagggccggttcaccatctcccgggacaacgccaagaacaccctgtttctgcagatgacctccctgcggagcgaggacaccgctatgtactactgcgccagacggggcctgctgctggactattggggccagggcaccaccctgaccgtctcctctgcctccaccaagggcccctccgtgttccctctggccccctccctggaatcctccggcagcgccaaggccgctgccatcggcatcgacctgggcaccacctacagctgcgtgggcgtgttccagcacggcaaggtggaaatcattgccaacgaccagggcaaccggatcaccccctcctacgtggccttcaccgacaccgagagactgatcggcgacgccgccaagaaccaggtggccctgaacccccagaacaccgtgttcgacgccaagcggctgatcggccggaagtttggcgacgccgtggtgcagtccgacatgaagcactggccatttcaggtcatcaacgacggcgacaagcccaaggtgcaggtctcctacaagggcgagacaaaggccttctaccccgaagagatctcctccatggtgctgaccaagatgaaggaaatcgccgaggcctacctgggctaccccgtgaccaacgccgtgatcaccgtgcccgcctacttcaacgacgcccagcggcaggccaccaaggatgctggcgtgatcgccggcctgaacgtgctgcggatcatcaacgagcccaccgccgctgctatcgcctacggcctggatagaaccggcaagggcgagcggaacgtgctgatcttcgacctgggcggcggcaccttcgacgtgtccatcctgaccatcgacgacggcatcttcgaagtgaaggccaccgctggcgacacccacctgggcggcgaggacttcgacaaccggctggtgaaccacttcgtggaagagttcaagcggaagcacaagaaggacatctcccagaacaagcgggccgtgcggcggctgagaaccgcctgcgagagagccaagcggaccctgtccagctccacccaggcctccctggaaatcgactccctgttcgagggcatcgacttctacacctccatcacccgggccagattcgaggaactgtgctccgacctgttccggtccaccctggaacccgtggaaaaggccctgcgggacgccaagctggacaaggcccagatccacgacctggtgctggtcggcggctccacaagaatccctaaggtgcagaagctgctgcaggacttcttcaacggccgggacctgcagcggtccatcaaccctgatgaggccgtggcctacggcgctgccgtgcaggctgctatcctgatgggcgacaagtccgagaacgtgcaggacctgctgctgctggacgtggcccctctgtccctgggcctggaaaccgctggcggcgtgatgaccgccctgatcaagcggaactccatcatccccaccaagcagacccagatcttcaccacctactccgacaaccagcccggcgtgctgatccaggtgtacgagggcgagagggccatgaccaaggacaacaacctgctgggcagattcgagctgtccggcatcccccctgcccctagaggcgtgccccagatcgaagtgaccttcgacatcgatgccaacggcatcctgcacgtgaccgccaccgacaagtccaccggcaaggccaacaagatcaccatcaccaacgacaagggccggctgtccaaagaagagatcgagcggatggtgcaggaagccgagaagtacaaggccgaggacgaggtgcagcgcgagagagtgtctgccaagaacgccctggaatcctacgccttcaacatgaagtccgccgtggaagatgagggcctgaagggcaagatctccgaggccgacaagaaaaaggtgctggacaagtgccaggaagtgatctcctggctggacgccaacaccctggccgagaaggacgagttcgagcacaagcggaaagaactggaacaagtctgcaaccccatcatctccggcctgtaccagggcgctggcggacctggacctggcggatttggcgctcagggccctaagggcggctctggctctggccccaccatcgaagaggtggactgatag

SEQ ID NO: 59 - protein, 927 aa, Artificial sequence
  Amino acid sequence of RBB004, Figure 21C
MGWSCIILFLVATATGVHSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPSLESSGSAKAAAIGIDLGTTYSCVGVFQHGKVEIIANDQGNRITPSYVAFTDTERLIGDAAKNQVALNPQNTVFDAKRLIGRKFGDAVVQSDMKHWPFQVINDGDKPKVQVSYKGETKAFYPEEISSMVLTKMKEIAEAYLGYPVTNAVITVPAYFNDAQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLQRSINPDEAVAYGAAVQAAILMGDKSENVQDLLLLDVAPLSLGLETAGGVMTALIKRNSIIPTKQTQIFTTYSDNQPGVLIQVYEGERAMTKDNNLLGRFELSGIPPAPRGVPQIEVTFDIDANGILHVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD